(19) United States
(12) Patent Application Publication
(10) Pub. No.: US 2020/0392138 A1
(43) Pub. Date: Dec. 17, 2020

(54) PESTICIDALLY ACTIVE MESOIONIC HETEROCYCLIC COMPOUNDS

(71) Applicant: Syngenta Participations AG, Basel (CH)

(72) Inventors: Pierre Joseph Marcel JUNG, Stein (CH); Raphael DUMEUNIER, Stein (CH); Julien Daniel Henri GAGNEPAIN, Stein (CH); André STOLLER, Stein (CH); Stefano RENDINE, Stein (CH)

(73) Assignee: Syngenta Participations AG, Basel (CH)

(21) Appl. No.: 16/772,555

(22) PCT Filed: Dec. 7, 2018
(86) PCT No.: PCT/EP2018/084032

§ 371 Date: Jun. 12, 2020

(30) Foreign Application Priority Data
Dec. 13, 2017 (EP) 172070054

(51) Int. Cl. C07D 487/04; A01N 43/90

(52) U.S. Cl. CPC C07D 487/04; A01N 43/90

(57) ABSTRACT

A compound of formula I, (I), wherein the substituents are as defined in claim (1), and the agrochemically acceptable salts, stereoisomers, enantiomers, tautomers and N-oxides of those compounds, can be used as insecticides.

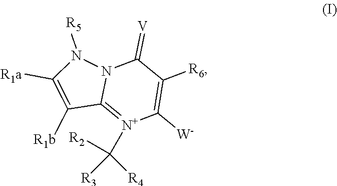

The present invention relates to pesticidally active, in particular insecticidally active mesoionics heterocyclic compounds, to compositions comprising those compounds, and to their use for controlling animal pests (including arthropods and in particular insects or representatives of the order lepidoptera and hemiptera).

Mesoionics heterocyclic compounds with pesticidal action are known and described, for example, in WO09099929, WO11017334, WO11017347, WO11017342, WO12092115, WO12106495, WO12136724, WO14033244, WO14202582, WO14167084, WO16055431, WO16171053 and WO17093214.

It has now been found further mesoionics heterocyclic compounds with pesticidal activity. The present invention accordingly, in a first aspect, relates to a compound of formula I,

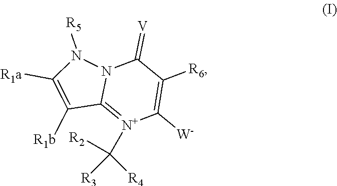

wherein

W is S or O;

V is S or O;

R₁ₐ and R_{1b} are, independently, hydrogen, halogen, amino, hydroxyl, C₁-C₆alkyl, C₁-C₆ haloalkyl, C₁-C₆haloalkoxy, C₁-C₆ alkoxy, or cyano;

R₂ is hydrogen, halogen, hydroxyl, amino, cyano, C₁-C₆ alkyl, mono- or poly-substituted C₁-C₆ alkyl wherein the substituent is independently selected from the group consisting of halogen, hydroxyl, amino, cyano, nitro, C₁-C₆ haloalkoxy, C₁-C₆ alkoxy, triazole, pyrazole, imidazole and tetrazole, wherein said triazole, pyrazole, imidazole and tetrazole can be mono- or polysubstituted by substituents independently selected from the group consisting of halogen, C₁-C₄ alkyl, C₁-C₄alkoxy, C₁-C₄ haloalkyl and cyano;

R₃ is hydrogen or C₁-C₆ alkyl;

R₄ is hydrogen or a 5 or 6 membered heteroaromatic ring Y, optionally independently substituted with a substituent from the group selected from U, wherein Y is a ring selected from Y1 to Y29

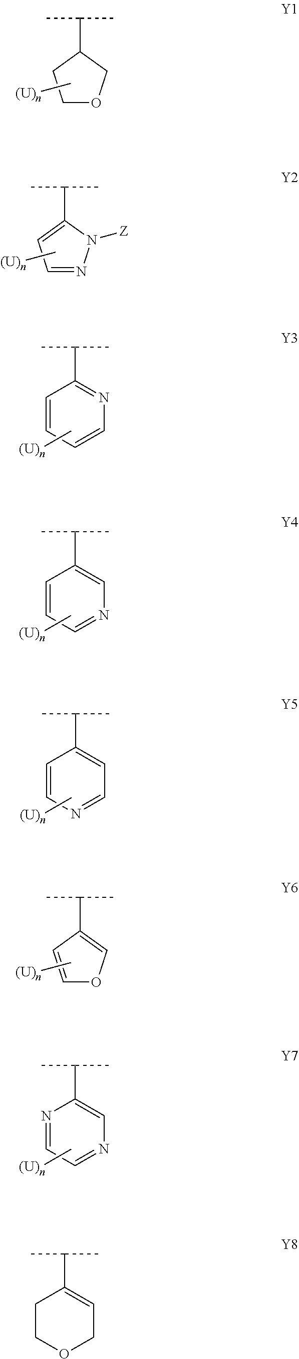

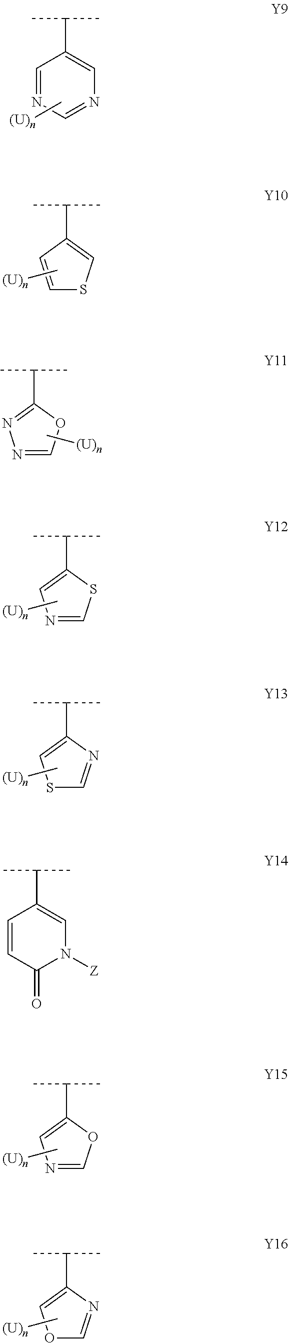

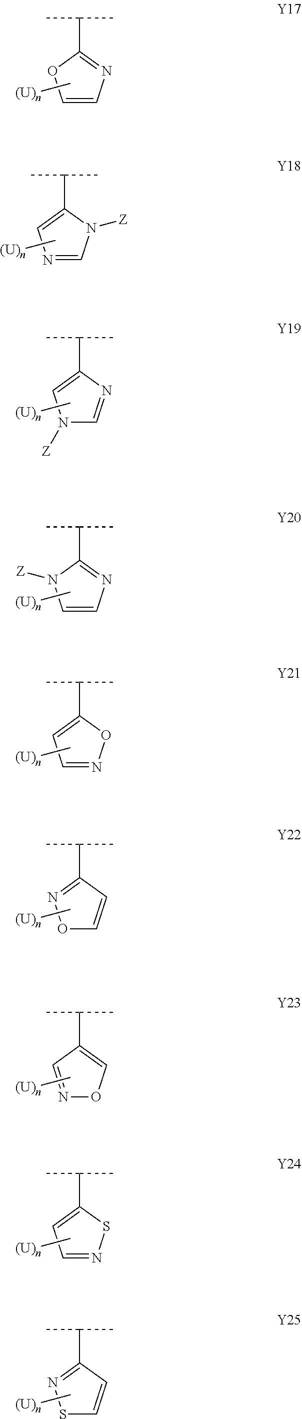

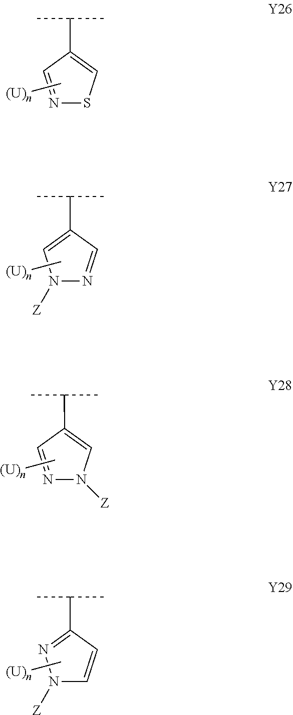

n is 0, 1, 2 or 3;

Z is hydrogen, cyano, nitro, hydroxyl, C₁-C₄ alkyl, C₁-C₄ haloalkyl, C₁-C₄ alkoxy or C₁-C₄ haloalkoxy;

U is independently selected from the group consisting of halogen, cyano, nitro, hydroxyl, amino, C₁-C₄ alkyl, C₁-C₄ haloalkyl, C₁-C₄ alkoxy, C₁-C₄ haloalkoxy, C₁-C₄ haloalkoxy-C₁-C₄ alkyl, C₁-C₄ alkoxy-C₁-C₄ alkyl, C₁-C₄alkylsulfanyl, C₁-C₄alkylsulfinyl, C₁-C₄ alkylsulfonyl, C₁-C₄haloalkylsulfanyl, C₁-C₄ haloalkylsulfinyl, C₁-C₄ haloalkylsulfonyl, and cyclopropyl;

R₅ is C₁-C₆ alkyl, C₃-C₆ cycloalkyl, C₁-C₆ haloalkyl, or C₁-C₆ alkoxy; or

R₅ is phenyl, the ring system of either can be mono- or polysubstituted by substituents independently selected from halogen, C₁-C₄alkyl, C₁-C₄ haloalkyl, C₁-C₄ alkoxy and C₁-C₄ haloalkoxy; and

R₆ is a 5 to 12 membered aromatic ring, which can be monocyclic or polycyclic, which ring system can be mono- or polysubstituted by substituents independently selected from the group U₂; or

R₆ is a 3 to 12 membered heteroaromatic ring or saturated or partially saturated heterocyclic ring, each of which ring system can be monocyclic or polycyclic, which ring system can contain 1 to 4 hetero atoms selected from the group consisting of nitrogen, oxygen and sulfur, with the proviso that each ring system cannot contain more than 2 oxygen atoms or more than 2 sulfur atoms, wherein the nitrogen heteroatom can be substituted by Z and said 3 to 12-membered ring system can be mono- or polysubstituted by substituents independently selected from the group U₂; or

R₆ is hydrogen, amino, halogen, cyano, C₁-C₆ haloalkoxy, C₁-C₆ alkoxy, C₁-C₄alkylsulfanyl, C₁-C₄ alkylsulfinyl, C₁-C₄ alkylsulfonyl, C₁-C₄haloalkylsulfanyl, C₁-C₄haloalkylsulfinyl, C₁-C₄haloalkylsulfonyl, C₂-C₆ alkenyl, C₂-C₆ haloalkenyl, C₂-C₆ alkynyl, C₂-C₆ haloalkynyl, benzyl optionally mono- or poly-substituted by a halogen (in case of polysubstitution, can be the same of different) or —C(O)R₇. or

R₆ is C₁-C₆ alkyl, which is optionally mono- or polysubstituted by substituents independently selected from the group U₃, or

R₆ is C₃-C₆ cycloalkyl, which is optionally mono- or polysubstituted by substituents independently selected from the group U; wherein

U₂ is halogen, nitro, cyano, amino, hydroxyl, —SCN, —CO₂H, C₁-C₆alkyl, C₃-C₆cycloalkyl, C₃-C₆ halocycloalkyl, C₃-C₆ cycloalkyl-C₁-C₄ alkyl, C₃-C₆ halocycloalkyl-C₁-C₄ alkyl, C₁-C₆ haloalkyl, C₁-C₆ alkoxy, C₁-C₄ alkoxy-C₁-C₄alkyl, C₁-C₄ alkoxy-C₁-C₄ alkoxy, cyano-C₁-C₄alkyl, cyano-C₁-C₄ haloalkyl, C₂-C₆ alkenyl, C₂-C₆ haloalkenyl, C₂-C₆ alkynyl, C₂-C₆ haloalkynyl, C₁-C₆ haloalkoxy, C₁-C₄ haloalkoxy-C₁-C₄alkyl, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, C₁-C₆alkylcarbonyl, C₁-C₆alkoxycarbonyl, C₁-C₆ haloalkylcarbonyl, C₁-C₆ haloalkoxycarbonyl, (C₁-C₆ alkyl)NH, (C₁-C₆ alkyl)₂N, (C₃-C₆ cycloalkyl)NH, (C₃-C₆ cycloalkyl)₂N, C₁-C₆ alkylcarbonylamino, C₃-C₆ cycloalkylcarbonylamino, C₁-C₆ haloalkylcarbonylamino, C₃-C₆ halocycloalkylcarbonylamino, C₁-C₆ alkylaminocarbonyl, C₃-C₆ cycloalkylaminocarbonyl, C₁-C₆ haloalkylaminocarbonyl, C₃-C₆ halocycloalkylaminocarbonyl, C₃-C₆ cycloalkylcarbonyl, C₃-C₆ halocycloalkylcarbonyl, —SF₅ or —C(O)NH₂;

U₃ is halogen, nitro, cyano, amino, hydroxyl, C₁-C₆alkyl, C₃-C₆ cycloalkyl, C₃-C₆ halocycloalkyl, C₃-C₆ cycloalkyl-C₁-C₄ alkyl, C₃-C₆ halocycloalkyl-C₁-C₄alkyl, C₁-C₆ haloalkyl, C₁-C₆ alkoxy, C₁-C₄ alkoxy-C₁-C₄ alkyl, C₁-C₄ alkoxy-C₁-C₄ alkoxy, cyano-C₁-C₄alkyl, cyano-C₁-C₄ haloalkyl, C₂-C₆ alkenyl, C₂-C₆ haloalkenyl, C₂-C₆ alkynyl, C₂-C₆ haloalkynyl, C₁-C₆ haloalkoxy, C₁-C₄ haloalkoxy-C₁-C₄alkyl, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, C₁-C₆ alkylcarbonyl, C₁-C₆ alkoxycarbonyl, C₁-C₆ haloalkylcarbonyl or C₁-C₆ haloalkoxycarbonyl; or

U₃ is a 5 to 6 membered aromatic ring, heteroaromatic ring, or saturated or partially saturated carbocyclic or heterocyclic ring (wherein the heteroatomatic and heterocyclic rings can contain 1 to 4 hetero atoms selected from the group consisting of nitrogen substituted or not, oxygen and sulfur, with the proviso that each ring system cannot contain more than 2 oxygen atoms or more than 2 sulfur atoms), wherein the said 5 to 6-membered ring system can be mono- or polysubstituted by substituents independently selected from the group U; and

R₇ is hydrogen, amino, halogen, cyano, C₁-C₆ alkyl, C₁-C₆ haloalkyl, C₁-C₆ alkoxy, C₁-C₆ haloalkoxy, C₁-C₄ haloalkoxy C₁-C₄ alkyl, C₁-C₆ alkoxy-C₁-C₆ alkyl, C₂-C₆ alkenyl, C₂-C₆ haloalkenyl, C₂-C₆ alkynyl or C₂-C₆ haloalkynyl; or

R₇ is a 5 to 6 membered aromatic ring, heteroaromatic ring, or saturated or partially saturated carbocyclic or heterocyclic (wherein the heteroatomatic and heterocyclic rings can can contain 1 to 4 hetero atoms selected from the group consisting of nitrogen substituted or not, oxygen and sulfur, with the proviso that each ring system cannot contain more than 2 oxygen atoms and more than 2 sulfur atoms), wherein the said 5 to 6-membered ring system can be mono- or polysubstituted by substituents independently selected from the group U; or an agrochemically acceptable salt, stereoisomer, enantiomer, tautomer or N-oxide thereof.

Compounds of formula I which have at least one basic centre can form, for example, acid addition salts, for example with strong inorganic acids such as mineral acids, for example perchloric acid, sulfuric acid, nitric acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄-alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The compounds of formula I are mesoionic compounds (also known as inner salts or zwitterions), which are understood to be compounds that are neutral but carry a formal positive and a negative charge on different atoms within the compounds. There are literature papers that have described these types of compounds, such as, for example Tetrahedron (1985), 41(12), 2239-329 or Tetrahedron 69 (2013) 4146-4159. Examples of mesoionics of formula I could be described by the following structures:

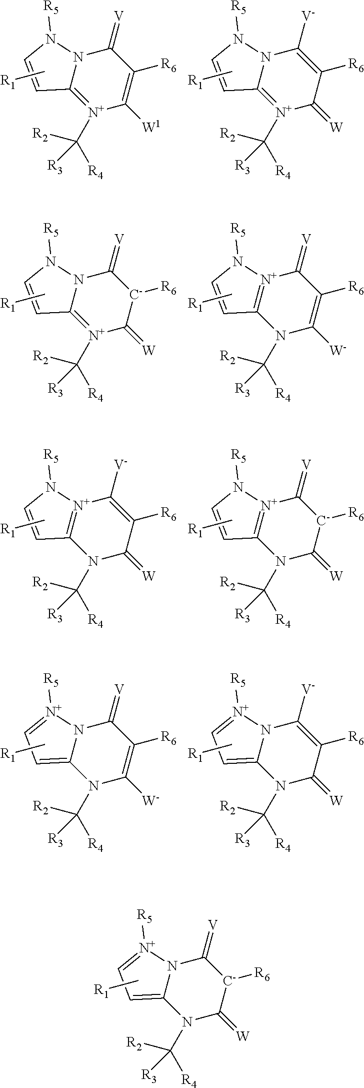

Accordingly, compounds according to the present invention can be represented by any one of the charge distribution above.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

A haloalkyl group is an alkyl group having one or more independently selected halogen atoms on the alkyl group. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 222-trichloroethyl.

An alkoxy group is an alkyl group connected to an oxygen atom, wherein the alkoxy group is connected to the rest of the compound via the oxygen atom. Alkoxy is, for example, methoxy, ethoxy, propoxy, i-propoxy, n-butoxy, isobutoxy, sec-butoxy and tert-butoxy and also the isomeric pentyloxy and hexyloxy radicals.

A cycloalkyl group has at least three carbon atoms in a ring, for example cyclopropyl, cyclobutyl, cyclopentyl and cyclohexyl, preferably cyclopropyl.

A haloalkoxy group is an alkoxy group having one or more independently selected halogen atoms on the alkyl group of the alkoxy group. Haloalkoxy is, for example, difluoromethoxy, trifluoromethoxy or 2,2,2-trifluoroethoxy.

An alkoxyalkyl group has one or more alkoxy groups and an alkyl group, wherein the alkoxy groups are in a chain with one of the oxygen atoms of the alkoxy chain connected to the alkyl group, which alkoxyalkyl group is connected to the rest of the compound via a carbon atom of the alkyl group. Alkoxyalkyl is, for example, methoxymethyl, methoxyethyl, ethoxymethyl, ethoxyethyl, n-propoxymethyl, n-propoxyethyl, isopropoxymethyl, isopropoxyethyl or a dialkoxyalkyl group such as for example CH₃₀CH₂CH₂OCH₂—.

A haloalkoxyalkyl group is an alkoxyalkyl group having one or more independently selected halogen atoms on the alkoxyalkyl group (for example the halogenation can be on the carbon atoms forming part of the alkyl and/or any one of the alkoxy group). Examples of haloalkoxyalkyl are, trifluoromethyloxymethyl, trifluoromethyloxyethyl, methoxyfluromethyl, trifluoroethyloxymethyl or a dihaloalkoxyalkyl group such as for example CF₃₀CH₂CH₂OCH₂—, CH₃₀CH₂CF₂₀CH₂—, C(Cl)F₂OCH₂CH₂OCH₂—, and CH₃OCH₂C(Cl)₂OCH₂—.

An alkylcarbonyl group is an alkyl group connected to a carbonyl group, which alkylcarbonyl group is connected to the rest of the compound via the carbon atom of the carbonyl moiety. Examples are CH₃C(O)—, and (CH₃)₂CHC(O)—.

A cycloalkylcarbonyl group is a cycloalkyl group connected to a carbonyl group, which cycloalkylcarbonyl group is connected to the rest of the compound via the carbon atom of the carbonyl moiety. Examples are cyclopropylC(O)—, and cyclobutylC(O)—.

A cycloalkylalkyl group is a cycloalkyl group connected to an alkyl group, which cycloalkylalkyl group is connected to the rest of the compound via a carbon atom of the alkyl group. Examples are -CyclopropylCH2-, and Cyclopropyl(CH₃)CH—.

A haloalkylcarbonyl group is an alkylcarbonyl group, wherein the alkyl group has one or more halogen atoms, which haloalkylcarbonyl group is connected to the rest of the compound via the carbon atom of the carbonyl moiety. An example of such is CF₃C(O)—.

An alkoxycarbonyl group is an alkoxy group connected to the carbon atom of a carbonyl group via the oxygen of the alkoxy group, which alkoxycarbonyl group is connected to the rest of the compound via the carbon atom of the carbonyl group. An example is CH₃₀C(O)—.

An haloalkoxycarbonyl group is an alkoxycarbonyl group wherein the alkoxy group is halogenated by one or more independently selected halogen atoms, for example, CF₃₀C(O)—.

An alkylcarbonylamino group is an alkylcarbonyl group connected to the nitrogen atom of an amino group via the carbon atom of the carbonyl group, which alkylcarbonylamino group is connected to the rest of the compound via the nitrogen atom of the amino group, such as for example CH₃C(O)NH—.

A cycloalkylcarbonylamino group is a cycloalkylcarbonyl group connected to the nitrogen atom of an amino group via the carbon atom of the carbonyl group, which cycloalkylcarbonylamino is connected to the rest of the compound via the nitrogen atom of the amino group, such as for example, cyclopropylC(O)NH—.

An alkylaminocarbonyl group has an alkyl group, an amino group and a carbonyl group, wherein a carbon atom of the the alkyl group is connected to a nitrogen atom of the amino group and then a nitrogen atom of the amino group is connected to the carbon atom of the carbonyl group, which alkylaminocarbonyl is connected to the rest of the compound via the carbon atom of the carbonyl group, such as for example, CH₃NHC(O)—.

A cycloalkylaminocarbonyl group has a cycloalkyl group, an amino group and a carbonyl group, wherein a carbon atom of the cycloalkyl group is connected to a nitrogen atom of the amino group and then a nitrogen atom of the amino group is connected to the carbon atom of the carbonyl group, which cycloalkylaminocarbonyl is connected to the rest of the compound via the carbon atom of the carbonyl group, such as for example, cyclopropylNHC(O)—.

A haloalkylcarbonylamino group is an alkylcarbonylamino group having one or more independently selected halogen atoms on the alkyl group, which haloalkylcarbonylamino group is connected to the rest of the compound via the nitrogen atom of the amino group, such as for example, CFH₂C(O)NH—.

A halocycloalkylcarbonylamino group is an cycloalkylcarbonylamino group having one or more independently selected halogen atoms on the cycloalkyl group, which halocycloalkylcarbonylamino group is connected to the rest of the compound via the nitrogen atom of the amino group, such as for example, 2-fluoro-cyclopropylC(O)NH—.

A haloalkylaminocarbonyl group is an alkylaminocarbonyl group having one or more independently selected halogen atoms on the alkyl group, which haloalkylaminocarbonyl is connected to the rest of the compound via the carbon atom of the carbonyl group, such as for example, CFH₂NHC(O)—.

A halocycloalkylaminocarbonyl group is a cycloalkylaminocarbonyl group having one or more independently selected halogen atoms on the cycloalkyl group, which halocycloalkylaminocarbonyl is connected to the rest of the compound via the carbon atom of the carbonyl group, such as for example 2-fluorocyclopropylNHC(O)—.

In the context of this invention “mono- to poly-substituted” in the definition of the substituents, means typically, depending on the chemical structure of the substituents, generally mono-substituted to seven-times substituted, preferably mono-substituted to five-times substituted, more preferably mono-, di- or tri-substituted.

As used herein, the term “C₂-C₆ alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₂-C₄alkynyl” and “C₂-C₃alkynyl” are to be construed accordingly. Examples of C₂-C₆alkynyl include, but are not limited to, ethynyl, prop-1-ynyl, but-1-ynyl and but-2-ynyl.

As used herein, the term “C₂-C₆ alkenyl” refers to a straight or branched hydrocarbon chain radical group consisting solely of carbon and hydrogen atoms, containing at least one double bond, having from two to six carbon atoms, and which is attached to the rest of the molecule by a single bond. The term “C₂-C₄alkenyl” and “C₂-C₃alkenyl” are to be defined accordingly. Examples of C₂-C₆ alkenyl include, but are not limited to prop-1-enyl, but-1-enyl and but-2-enyl.

Alkylsulfanyl is for example methylsulfanyl, ethylsulfanyl, propylsulfanyl, isopropylsulfanyl, butylsulfanyl, pentylsulfanyl and hexylsulfanyl.

Alkylsulfinyl is for example methylsulfinyl, ethylsulfinyl, propylsulfinyl, isopropylsulfinyl, a butylsulfinyl, pentylsulfinyl or hexylsulfinyl.

Alkylsulfonyl is for example methylsulfonyl, ethylsulfonyl, propylsulfonyl, isopropylsulfonyl, butylsulfonyl, pentylsulfonyl or hexylsulfonyl.

Haloalkylsulfanyl is for example difluoromethylsulfanyl, trifluoromethylsulfanyl, 2,2,2-trifluoroethylsulfanyl or pentafluoroethylsulfanyl.

Haloalkylsulfinyl is for example difluoromethylsulfinyl, trifluoromethylsulfinyl, 2,2,2-trifluoroethylsulfinyl or pentafluoroethylsulfinyl.

Haloalkylsulfonyl is for example difluoromethylsulfonyl, trifluoromethylsulfonyl, 2,2,2-trifluoroethylsulfonyl or pentafluoroethylsulfonyl.

Examples of a 5 to 12 membered aromatic ring system, which can be monocyclic or polycyclic, include phenyl, naphthyl, anthracenyl and biphenyl; preferred are phenyl, naphthyl, and biphenyl.

Examples of a 3 to 12 membered heteroaromatic ring system, which can be monocyclic or polycyclic, include pyridyl, pyrimidyl, pyrrolyl, pyrazolyl, furyl, thienyl, imidazolyl, isoxazolyl, oxazolyl, thiazolyl, isothiazolyl, triazolyl, oxadiazolyl, thiadiazolyl, tetrazolyl, pyrazinyl, pyridazinyl, triazinyl, pyranyl, quinazolinyl, isoquinolinyl, indolizinyl, isobenzofuranylnaphthyridinyl, quinoxalinyl, isochinolinyl, cinnolinyl, phthalazinyl, benzothiazolyl, benzoxazolyl, benzotriazolyl, indazolyl, indolyl, tetrahydroquinolynyl, benzofuryl, benzisofuryl, benzothienyl, benzisothienyl, isoindolyl, naphthyridinyl, benzisothiazolyl, benzisoxazolyl, benzoxazolyl, benzotriazinyl, purinyl, chinazolinyl, chinoxalinyl, teridinyl, Indolizinyl, phenylpyridyl, and pyridylphenyl; preferred are pyridyl, pyrimidyl, phenylpyridyl, pyridylphenyl, and thienyl.

Examples of a 3 to 12 membered saturated or partially saturated heterocyclic ring system, which can be monocyclic or polycyclic, include dihydropyranyl, tetrahydrofuryl, tetrahydrofuranyl, tetrahydrothienyl, pyrrolidinyl, pyrrolidinyl, isoxazolidinyl, isothiazolidinyl, pyrazolidinyl, oxazolidinyl, thiazolidinyl, imidazolidinyl, oxadiazolidinyl, thiadiazolidinyl, dihydrofuryl, dihydrothienyl, pyrrolinyl, isoxazolinyl, dihydropyrazolyl, dihydrooxazolyl, piperidinyl, dioxanyl, tetrahydropyranyl, tetrahydrothienyl, hexahydropyridazinyl, hexahydropyrimidinyl, oxiranyl, and piperazinyl; preferred is tetrahydrofuryl.

Example of a 5 to 6 membered aromatic ring system includes phenyl.

Examples of a 5 to 6 membered heteroaromatic ring system include pyridyl, pyrimidyl, pyrrolyl, pyrazolyl, furyl, thienyl, imidazolyl, isoxazolyl, oxazolyl, thiazolyl, isothiazolyl, triazolyl, oxadiazolyl, thiadiazolyl, tetrazolyl, pyrazinyl, pyridazinyl, triazinyl, and pyranyl; preferred are pyridyl, pyrimidyl, and thienyl.

Examples of a 5 to 6 membered saturated or partially saturated carbocyclic or heterocyclic ring system include dihydropyranyl, tetrahydrofuryl, tetrahydrofuranyl, tetrahydrothienyl, pyrrolidinyl, pyrrolidinyl, isoxazolidinyl, isothiazolidinyl, pyrazolidinyl, oxazolidinyl, thiazolidinyl, imidazolidinyl, oxadiazolidinyl, thiadiazolidinyl, dihydrofuryl, dihydrothienyl, pyrrolinyl, isoxazolinyl, dihydropyrazolyl, dihydrooxazolyl, piperidinyl, dioxanyl, tetrahydropyranyl, tetrahydrothienyl, hexahydropyridazinyl, hexahydropyrimidinyl, and piperazinyl; preferred is tetrahydrofuryl.

Polycyclic as used herein refers to fused cyclic rings, and substituted cyclic rings, in which the substituent is another cyclic ring (such as an aryl or heteroaryl ring). An example of a fused ring is naphthyl or benzisoxazolyl or benzoxazolyl, whereas an example of a substituted ring is biphenyl or 2-phenylpyridyl or 2-pyridylphenyl. Whenever a polycyclic ring is indicated to be substituted, unless specific substitution position is indicated, the substituent(s) can be on the same or different substitutable position(s) on the same or different rings.

A “ring system” as used herein refers in entirety to the ring substitutent whether monocyclic or polycyclic. For example, in the instance of “R₅ being a 3 to 12 membered heteroaromatic ring, which ring system can contain 1 to 4 heteroatoms selected from the group consisting of nitrogen, oxygen and sulfur, with the proviso that each ring cannot contain more than 2 oxygen atoms or more than 2 sulfur atoms, wherein the nitrogen heteroatom can be substituted by Z and said 3 to 12-membered ring system can be mono- or polysubstituted by substituents independently selected from the group U₂”, the ring system refers to the fact that only 1 to 4 heteroatoms can be present in total and not per ring.

Whenever it is written that a group or ring can be substituted, it means that the group or ring is optionally substituted.

The compounds of formula I according to the invention also include hydrates which may be formed during the salt formation.

In an embodiment, independent of the different aspects or embodiments, n is 0 or 1.

In an embodiment, independent of the different aspects or embodiments, Z is hydrogen, cyano, nitro, hydroxyl, C₁-C₄alkyl, or C₁-C₄alkoxy; preferably Z is hydrogen, C₁-C₄alkyl or C₁-C₄alkoxy; more preferably Z is hydrogen, or C₁-C₄ alkyl; especially Z is hydrogen, methyl or ethyl.

In an embodiment, independent of the different aspects or embodiments, U is independently selected from the group consisting of halogen, hydroxyl, amino, C₁-C₄ alkyl, C₁-C₄ haloalkyl, C₁-C₄ alkoxyl, C₁-C₄ haloalkoxyl, cyano, C₁-C₄ alkylsulfanyl and C₁-C₄ alkylsulfonyl; preferably U is independently selected from the group consisting of halogen, C₁-C₄ haloalkyl, C₁-C₄alkoxyl, cyano, C₁-C₄ alkylsulfanyl and C₁-C₄ alkylsulfonyl; more preferably U is independently selected from the group consisting of halogen, trifluoromethyl, methoxy, cyano, methylsulfanyl and methylsulfonyl; especially U is independently selected from the group consisting of chlorine and trifluoromethyl.

In an embodiment, independent of the different aspects or embodiments, U₂ is halogen, cyano, C₁-C₆ alkyl, C₁-C₆ alkoxy or C₁-C₆ haloalkyl; preferably U₂ is halogen, cyano, C₁-C₄ alkyl, C₃-C₆ cycloalkyl, C₁-C₄ haloalkyl or C₁-C₄ alkoxy; especially U₂ is chloro, fluoro, cyano, methyl, cyclopropyl, trifluoromethyl or methoxy.

In an embodiment, independent of the different aspects or embodiments, U₃ is halogen, nitro, cyano, C₁-C₆ alkyl, C₃-C₆ cycloalkyl, C₁-C₆ haloalkyl, C₁-C₆ alkoxy, C₁-C₆ haloalkoxy, C₁-C₆ haloalkylsulfanyl or a phenyl, said phenyl is optionally mono- or polysubstituted by substituents independently selected from the group U; preferably U₃ is halogen, C₁-C₆ haloalkyl, C₁-C₆ haloalkoxy, C₁-C₆ haloalkylsulfanyl or phenyl; especially U₃ is chloro, fluoro, trifluoromethyl, trifluoromethoxy, trifluoromethylsulfanyl or phenyl.

In an embodiment, independent of the different aspects or embodiments, W is O and V is either O or S; preferably both W and V are O.

In an embodiment, independent of the different aspects or embodiments, R₁ₐ is selected from hydrogen and C₁-C₆ alkyl; preferably R₁ₐ is selected from hydrogen and C₁-C₄ alkyl; more preferably R₁ₐ is selected from hydrogen, methyl, ethyl, propyl and isopropyl; especially R₁ₐ is either hydrogen or methyl.

In an embodiment, independent of the different aspects or embodiments, R_{1b} is selected from hydrogen and C₁-C₆ alkyl; preferably R_{1b} is selected from hydrogen and C₁-C₄ alkyl; more preferably R_{1b} is selected from hydrogen, methyl, ethyl, propyl and isopropyl; especially R_{1b} is either hydrogen or methyl.

In an embodiment, independent of the different aspects or embodiments, R₁ₐ and R_{1b} are independently selected from hydrogen and methyl; preferably R₁ₐ and R_{1b} are each hydrogen.

In an embodiment, independent of the different aspects or embodiments, R₂ is selected from hydrogen and mono- or poly-substituted C₁-C₆ alkyl, where the substituent is independently selected from the group consisting of halogen, cyano, triazole and imidazole, wherein said triazole and imidazole can be mono- or polysubstituted by halogen, which halogen, in case of polysubstitution, can be the same or different; preferably R₂ is selected from hydrogen and mono- or poly-substituted C₁-C₄ alkyl, where the substituent is independently selected from the group consisting of halogen, cyano, triazole and imidazole, wherein said triazole and imidazole can be mono- or polysubstituted by halogen, which halogen, in case of polysubstitution, can be the same or different; more preferably R₂ is selected from hydrogen, trifluoromethyl, trifluoroethyl, cyanomethyl, triazole and imidazole, wherein said triazole and imidazole is optionally substituted by chlorine; especially R₂ is selected from hydrogen, trifluoromethyl, trifluoroethyl and cyanomethyl.

In an embodiment, independent of the different aspects or embodiments, R₃ is hydrogen or C₁-C₄ alkyl; preferably R₃ is hydrogen, methyl, ethyl, propyl or isopropyl; more preferably R₃ is hydrogen.

In an embodiment, independent of the different aspects or embodiments, R₄ is hydrogen or a 5 or 6 membered heteroaromatic ring selected from Y1, Y3, Y4, Y5, Y7, Y9, Y12, Y18, Y21 and Y23, wherein Z is hydrogen or C₁-C₄ alkyl, U is selected from the group consisting of halogen, C₁-C₄ haloalkyl, C₁-C₄alkoxyl, cyano, C₁-C₄alkylsulfanyl and C₁-C₄ alkylsulfonyl, and n is 0 or 1; preferably wherein Z is hydrogen or methyl, U is selected from the group consisting of halogen, trifluoromethyl, methoxy, cyano, methylsulfanyl and methylsulfonyl, and n is 0 or 1; more preferably R₄ is hydrogen or a 5 or 6 membered heteroaromatic ring selected from Y4, Y9, and Y12, where U is selected from the group consisting of halogen, trifluoromethyl, methoxy, cyano, methylsulfanyl and methylsulfonyl, and n is 0 or 1; especially R₄ is hydrogen or a 5 or 6 membered heteroaromatic ring selected from Y4, Y9, and Y12, U is selected from the group consisting of halogen and trifluoromethyl, and n is 0 or 1.

In an embodiment, independent of the different aspects or embodiments, R₅ is C₁-C₆ alkyl, C₃-C₆ cycloalkyl, C₁-C₆ haloalkyl or phenyl; preferably R₅ is C₁-C₄ alkyl, C₃-C₄ cycloalkyl, C₁-C₄ haloalkyl or phenyl; more preferably R₅ is methyl, ethyl, isopropyl, trifluoromethyl, trifluoroethyl, cyclopropyl or phenyl; especially R₅ is methyl, ethyl, trifluoroethyl, cyclopropyl or phenyl; more especially R₅ is methyl, ethyl, trifluoroethyl or cyclopropyl.

In an embodiment, independent of the different aspects or embodiments, R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which C₁-C₄ alkyl is optionally mono- or poly-substituted by substituents independently selected from a halogen (in case of polysubstitution, can be the same or different) or C₃-C₆ cycloalkyl; preferably R₆ is hydrogen, iodine, bromine, chlorine, fluorine, —C(O)R₇ (wherein R₇ is C₁-C₄ haloalkyl or phenyl), phenyl optionally mono- or poly-substituted by the group consisting of halogen, trifluoromethyl, trifluoromethoxy and trifluoromethylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and trifluoromethyl, C₁-C₄ alkyl, which is optionally mono- or poly-substituted by substituents independently selected from chlorine and fluorine or cyclohexane; more preferably R₆ is hydrogen, iodine, —C(O)R₇ (wherein R₇ is trifluoromethyl or phenyl), phenyl optionally mono- or poly-substituted by the group consisting of halogen and trifluoromethyl, naphthyl optionally substituted by a halogen (in case of polysubstitution, can be the same or different), pyridylphenyl optionally mono- or poly-substituted by substituents independently selected from halogen and trifluoromethyl, or C₁-C₄ alkyl, which is optionally mono- or poly-substituted by substituents independently selected from chlorine and fluorine.

Preferred compounds of formula I are where W is O and V is either O or S; R₁ₐ is selected from hydrogen and C₁-C₆ alkyl; R_{1b} is selected from hydrogen and C₁-C₆ alkyl; R₂ is selected from hydrogen and mono- or poly-substituted C₁-C₆alkyl, where the substituent is independently selected from the group consisting of halogen, cyano, triazole and imidazole, wherein said triazole and imidazole can be mono- or polysubstituted by halogen, which halogen, in case of polysubstitution, can be the same or different; R₃ is hydrogen or C₁-C₄ alkyl; R₄ is hydrogen or a 5 or 6 membered heteroaromatic ring selected from Y1, Y3, Y4, Y5, Y7, Y9, Y12, Y18, Y21 and Y23, wherein Z is C₁-C₄ alkyl, U is selected from the group consisting of halogen, C₁-C₄ haloalkyl, C₁-C₄ alkoxyl, cyano, C₁-C₄ alkylsulfanyl and C₁-C₄ alkylsulfonyl, and n is 0 or 1; R₅ is C₁-C₆ alkyl, C₃-C₆ cycloalkyl, C₁-C₆ haloalkyl or phenyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which is optionally mono- or poly-substituted by substituents independently selected from a halogen (in case of polysubstitution, can be the same or different), or C₃-C₆ cycloalkyl.

Preferred compounds of formula I are compounds where W and V are each O; R₁ₐ is selected from hydrogen and C₁-C₄ alkyl; R_{1b} is selected from hydrogen and C₁-C₄ alkyl; R₂ is selected from hydrogen, and mono- or poly-substituted C₁-C₄alkyl, where the substituent is independently selected from the group consisting of halogen, cyano, triazole and imidazole, wherein said triazole and imidazole can be mono- or polysubstituted by halogen, which halogen, in case of polysubstitution, can be the same or different; R₃ is hydrogen, methyl, ethyl, propyl or isopropyl; R₄ is hydrogen or a 5 or 6 membered heteroaromatic ring selected from Y1, Y3, Y4, Y5, Y7, Y9, Y12, Y18, Y21 and Y23, wherein Z is methyl, U is selected from the group consisting of halogen, trifluoromethyl, methoxy, cyano, methylsulfanyl and methylsulfonyl, and n is 0 or 1; R₅ is C₁-C₄ alkyl, C₃-C₄ cycloalkyl, C₁-C₄ haloalkyl or phenyl; and R₆ is hydrogen, iodine, bromine, chlorine, fluorine, —C(O)R₇ (wherein R₇ is C₁-C₄ haloalkyl or phenyl), phenyl optionally mono- or poly-substituted by the group consisting of halogen, trifluoromethyl, trifluoromethoxy and trifluoromethylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and trifluoromethyl, C₁-C₄alkyl, which is optionally mono- or poly-substituted by substituents independently selected from chlorine and fluorine; or cyclohexane.

Preferred compounds of formula I are compounds where W and V are each O; R₁ₐ is selected from hydrogen, methyl, ethyl, propyl and isopropyl; R_{1b} is selected from hydrogen, methyl, ethyl, propyl and isopropyl; preferably R₂ is selected from hydrogen, trifluoromethyl, trifluoroethyl, cyanomethyl, triazole, and imidazole, wherein said triazole and imidazole is optionally substituted by chlorine; R₃ is hydrogen; R₄ is hydrogen or a 5 or 6 membered heteroaromatic ring selected from Y4, Y9, and Y12, where U is selected from the group consisting of halogen, trifluoromethyl, methoxy, cyano, methylsulfanyl, and methylsulfonyl, and n is 0, 1; R₅ is methyl, ethyl, isopropyl, trifluoromethyl, trifluoroethyl, cyclopropyl or phenyl; and R₆ is hydrogen, iodine, —C(O)R₇ (wherein R₇ is trifluoromethyl or phenyl), phenyl optionally mono- or poly-substituted by the group consisting of halogen, and trifluoromethyl, naphthyl optionally substituted by a halogen (in case of polysubstitution, can be the same or different), pyridylphenyl optionally mono- or poly-substituted by substituents independently selected from halogen, and trifluoromethyl, or C₁-C₄ alkyl, which is optionally mono- or poly-substituted by substituents independently selected from chlorine and fluorine.

Preferred compounds of formula I are compounds where W and V are each O; R₁ₐ is either hydrogen or methyl; R₁ is either hydrogen or methyl; R₂ is selected from hydrogen, trifluoromethyl, trifluoroethyl and cyanomethyl; R₃ is hydrogen; R₄ is hydrogen or a 5 or 6 membered heteroaromatic ring selected from Y4, Y9, and Y12, where U is selected from the group consisting of halogen, trifluoromethyl, methoxy, cyano, methylsulfanyl and methylsulfonyl, and n is 0 or 1; R₅ is methyl, ethyl, trifluoroethyl, cyclopropyl or phenyl; and R₆ is hydrogen, iodine, —C(O)R₇ (wherein R₇ is trifluoromethyl or phenyl), phenyl optionally mono- or poly-substituted by the group consisting of halogen and trifluoromethyl, naphthyl optionally substituted by a halogen (in case of polysubstitution, can be the same or different), pyridylphenyl optionally mono- or poly-substituted by substituents independently selected from halogen and trifluoromethyl, or C₁-C₄ alkyl, which is optionally mono- or poly-substituted by substituents independently selected from chlorine and fluorine.

Preferred compounds of formula I are compounds where W and V are each O; R₁ₐ and R_{1b} are each hydrogen; R₂ is selected from hydrogen, trifluoromethyl, trifluoroethyl and cyanomethyl; R₃ is hydrogen; R₄ is hydrogen or a 5 or 6 membered heteroaromatic ring selected from Y4, Y9, and Y12, U is selected from the group consisting of halogen and trifluoromethyl, and n is 0 or 1; R₅ is methyl, ethyl, trifluoroethyl, cyclopropyl or phenyl; and R₆ is hydrogen, iodine, —C(O)R₇ (wherein R₇ is trifluoromethyl or phenyl), phenyl optionally mono- or poly-substituted by the group consisting of halogen and trifluoromethyl, naphthyl optionally substituted by a halogen (in case of polysubstitution, can be the same or different), pyridylphenyl optionally mono- or poly-substituted by substituents independently selected from halogen and trifluoromethyl, or C₁-C₄ alkyl, which is optionally mono- or poly-substituted by substituents independently selected from chlorine and fluorine.

Preferred compounds of formula I are where W and V are each O, R₁ₐ and R_{1b} are each hydrogen; R₂ is selected from hydrogen, trifluoromethyl, trifluoroethyl, cyanomethyl, triazole and imidazole, wherein said triazole and imidazole is optionally substituted by chlorine; R₃ is hydrogen; R₄ is hydrogen or a 5 or 6 membered heteroaromatic ring selected from Y1, Y3, Y4, Y5, Y7, Y9, Y12, Y18, Y21 and Y23, wherein Z is C₁-C₄ alkyl, U is selected from the group consisting of halogen, C₁-C₄ haloalkyl, C₁-C₄ alkoxyl, cyano, C₁-C₄ alkylsulfanyl and C₁-C₄ alkylsulfonyl, and n is 0 or 1; R₅ is methyl, ethyl, trifluoroethyl or cyclopropyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which is optionally mono- or poly-substituted by substituents independently selected from a halogen (in case of polysubstitution, can be the same or different), or C₃-C₆ cycloalkyl.

Preferred compounds of formula I are compounds where W and V are each O, R₁ₐ and R_{1b} are each hydrogen; R₂ is selected from trifluoromethyl, trifluoroethyl and cyanomethyl; R₃ is hydrogen; R₄ is hydrogen; R₅ is methyl, ethyl, trifluoroethyl or cyclopropyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄alkyl, which is optionally mono- or poly-substituted by substituents independently selected from a halogen (in case of polysubstitution, can be the same or different), or C₃-C₆ cycloalkyl.

Preferred compounds of formula I are compounds where W and V are each O, R₁ₐ and R_{1b} are each hydrogen; R₂ is selected from hydrogen, trifluoromethyl, trifluoroethyl and cyanomethyl; R₃ is hydrogen; R₄ is hydrogen or a 5 or 6 membered heteroaromatic ring selected from Y1, Y3, Y4, Y5, Y7, Y9, Y12, Y18, Y21 and Y23, wherein Z is C₁-C₄ alkyl, U is selected from the group consisting of halogen, C₁-C₄ haloalkyl, C₁-C₄ alkoxyl, cyano, C₁-C₄ alkylsulfanyl and C₁-C₄ alkylsulfonyl, and n is 0 or 1; preferably wherein Z is methyl, U is selected from the group consisting of halogen, trifluoromethyl, methoxy, cyano, methylsulfanyl and methylsulfonyl, and n is 0 or 1; R₅ is methyl, ethyl, trifluoroethyl or cyclopropyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which is optionally mono- or poly-substituted by substituents independently selected from a halogen (in case of polysubstitution, can be the same or different), or C₃-C₆ cycloalkyl.

Whenever a substituent list (e.g. U or U₂ or U₃) is used in more than one substituent in the compound, the substituent list, in each case, is independently selected for each substituent (e.g. in the instance of U, it can be used independently in any one of the rings Y, and independently used for the ring R₇ (for example, it can be a halogen atom for ring Y1, cyano for ring Y3, and hydroxyl for the 5 to 6 membered aromatic ring R₇; or in the instance of U₂, it can be a substituent on the 5 to 12 membered aromatic ring R₆ and also on a 5 or 6 membered aromatic ring U₃; or in the instance of U₂, it can be independently used as a substituent on C₃-C₆ cycloalkyl R₆ or C₁-C₆ alkyl R₆, etc),

The compounds of the invention (including the intermediates) can be made by analogy methods known to those skilled in the art, for example, in WO9099929, WO11017334, WO11017347, WO11017342, WO12092115, WO12106495, WO12136724, WO14033244, WO14202582, WO14167084, WO16055431, WO16171053 and WO17093214.

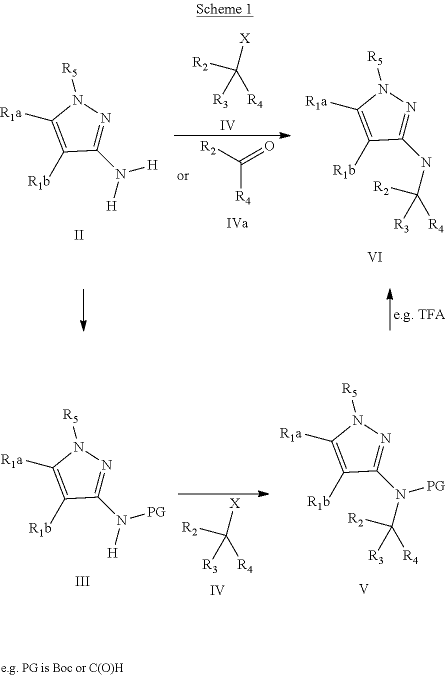

Compounds of formula VI wherein R₁ₐ, R_{1b}, R₂, R₄ and R₅ are as defined in formula I above and wherein R₃ is hydrogen, can be made by formation of the N—CHR₂R₄ bond via reductive amination with an carbonyl compound R₂R₄C(O) (formula IVa). Reductive amination may be achieved by treatment of the compounds of formula II with an carbonyl compound IVa and a reducing agent such as sodium cyanoborohydride. Such reactions can be carried out under well-established methods and various conditions could be used, described for example in Synthetic Organic Methodology: Comprehensive Organic Transformations, a Guide to Functional Group Preparations, Larock, R. C. 1989 p 421.

Compounds of formula VI wherein R₁ₐ, R_{1b}, R₂, R₃, R₄ and R₅ are as defined in formula I above may be achieved by alkylation. Treatment of the compounds of formula II with compound of formula IV wherein X is a leaving group, such as chloro, bromo, iodo, mesylate, triflate in presence of a base such as potassium carbonate in a solvent such as dimethylsulfoxide, acetonitrile, tetrahydrofuran, dimethylformamide or toluene could give compounds of formula VI wherein R₁ₐ, R_{1b}, R₂, R₃, R₄ and R₅ are as defined in formula I above. Such reactions can be carried out under well-established methods, described for example, EP 2944637, Bioorganic & Medicinal Chemistry Letters, 23(23), 6467-6473; 2013 or Journal of Medicinal Chemistry, 51(23), 7370-7379; 2008.

Alternatively, the sequence to prepare compounds of formula VI from compounds of formula II, may involve i. a selective acylation of compound II to form a compound of formula III, wherein R₁ₐ, R_{1b} and R₅ are as described under formula I above and wherein the acylation agent is for example di-tert-butyl dicarbonate (wherein PG is tert-butyloxycarbonyl), in a solvent, such as for example, tetrahydrofuran or dioxane; ii. alkylation of compound III with IV, wherein R₂, R₃ and R₄ are as described under formula I above and wherein X is a leaving group, such as halogen, preferably iodine, bromine or chlorine, in presence of a base, such as sodium carbonate, potassium carbonate or cesium carbonate, or sodium hydride, in a appropriate solvent such as for example N,N-dimethylformamide, N,N-dimethylacetamide or acetonitrile, to generate a compound of formula V, wherein R₁ₐ, R_{1b}, R₂, R₃, R₄ and R₅ are as defined in formula I above and wherein PG is for example tert-butyloxycarbonyl; and finally iii. deacylation of compound V to form the compound of formula VI, wherein R₁ₐ, R_{1b}, R₂, R₃, R₄ and R₅ are as defined in formula I above. When PG is for example tert-butyloxycarbonyl, conditions for the acyl group removal include, for example, treatment of compound V with hydrogen halide, in particular hydrogen chloride or hydrogen bromide, in solvents such as ethers (for example diethyl ether, tetrahydrofuran or dioxane) or acetic acid. Alternatively, compound V may also be treated with, for example, trifluoroacetic acid, in optional presence of an inert solvent, such as for example dichloromethane or chloroform, to form a compound of formula VI.

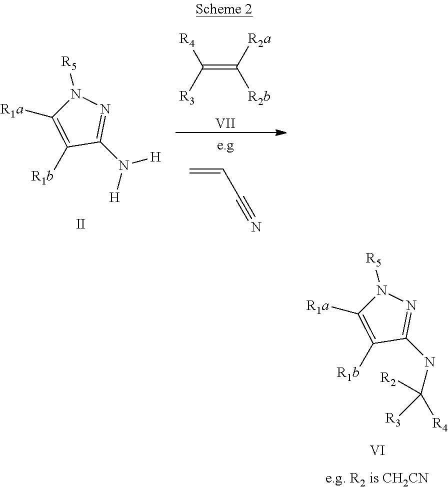

Compounds of formula VI, wherein R₁ₐ, R_{1b}, R₂, R₃, R₄ and R₅ are as defined in formula I above, can be prepared (as shown in scheme 2) by aza-Michael addition reaction with a compound of formula II. These reactions are well known to those skilled in the art and described in, for example, Synthesis 2008, (24), 3931-3936 and cited references, Tetrahedron 2011 67(20) p 3631-3637 and cited references. This type of reaction could be done in absence or presence of solvents or catalysts and many conditions were developed. For example, compounds of formula VI, wherein R₁ₐ, R_{1b}, R₂, R₃ and R₅ are as defined in formula I above and R₂ is CH₂CN, can be prepared by reaction of acrylonitrile in presence of a catalyst such as copper(II) acetate without solvent at temperature between 25° C. and 100° C., preferably at 80° C.

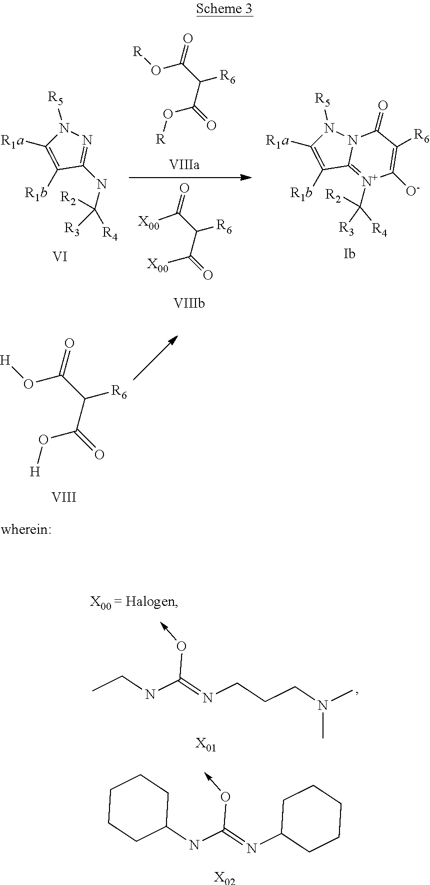

Compounds of formula Ib, wherein R₁ₐ, R_{1b}, R₂, R₃, R₄, R₅ and R₆ are as defined in formula I above, can be prepared (as shown in scheme 3) by reaction of compound of formula VI (wherein R₁ₐ, R_{1b}, R₂, R₃, R₄, and R₅ are as defined in formula I above) with a compound of formula VIIIa wherein R is aryl or alkyl, such as ethyl, phenyl or 1,3,5-trichlorophenyl; in inert solvents such as toluene or THF, at temperatures between 20° C. to reflux of the used solvent. By analogy, these methods are well known to those skilled in the art and described in, for example, Bulletin of the Chemical Society of Japan, 72(3), 503-509; 1999, Archiv der Pharmazie (Weinheim, Germany), 1991, 324(11), 863-6 or WO 2009099929.

Alternatively, Compounds of formula Ib, wherein R₁ₐ, R_{1b}, R₂, R₃, R₄, R₅ and R₆ are as defined in formula I above, can be prepared (as shown in scheme above) by activation of compound of formula VIII, wherein R₆ is as defined above, by methods known to those skilled in the art and described in, for example, Tetrahedron, 2005 61 (46) 10827-10852 or Tetrahedron, 2004 60(44) 10011-10018, to form the compound VIIIb, wherein R₆ is as defined above and wherein X₀₀ is halogen, preferably chlorine. For example, compounds VIIIb where X₀₀ is halogen, preferably chlorine, are formed by treatment of VIII with, for example, oxalyl chloride (COCl)₂ or thionyl chloride (SOCl₂), in the presence of catalytic quantities of N,N-dimethylformamide (DMF) in inert solvents such as methylene chloride (CH₂Cl₂) or tetrahydrofuran (THF) at temperatures between 20° C. to 100° C., preferably 25° C.

Alternatively, treatment of compounds of formula VIII with, for example, 1-ethyl-3-(3-dimethylaminopropyl)carbodiimide (EDC) or dicyclohexyl carbodiimide (DCC) will generate the compound VIIIb, wherein X₀₀ is X₀₁ or X₀₂ respectively, in an inert solvent, such as pyridine or tetrahydrofurane (THF), optionally in the presence of a base, such as triethylamine, at temperatures between 25-180° C.; followed by treatment of the compound VIIIb with a compound of formula VI, wherein R₁ₐ, R_{1b}, R₂, R₃, R₄ and R₅ are as defined in formula I above, optionally in the presence of a base, such as triethylamine or pyridine, in an inert solvent such as dichloromethane, tetrahydrofuran, dioxane or toluene, at temperatures between 0 and 80° C., to form the compounds of formula Ib.

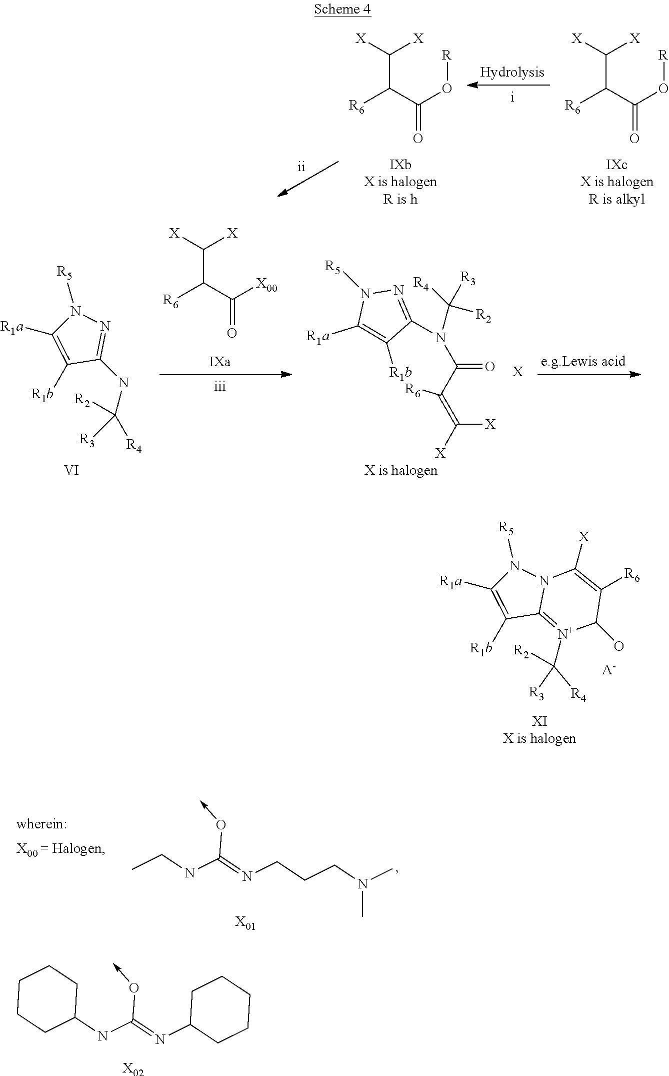

Compounds of the formula X, wherein R₁ₐ, R_{1b}, R₂, R₃, R₄, R₅ and R₆ are as defined in formula I above, may be prepared by:

i) Compound of formula IXb, wherein R₆ as defined in formula I above, may be prepared by reaction of a compound of formula IXc, wherein R is C₁-C₄ alkyl via hydrolysis. For instance, in the case where R is methyl or ethyl, the hydrolysis can be done with water and a base, such as potassium hydroxide or lithium hydroxide, in the absence or in the presence of a solvent or a mixture of solvents, such as, for instance, tetrahydrofurane or methanol. In the case where R is, for example, tert-butyl, the hydrolysis is done in the presence of acid, such as trifluoroacetic acid or hydrochloric acid. The reaction is carried out at a temperature of from −78° C. to +130° C., preferably from 0° C. to 120° C. This transformation is well known to persons skilled in the art and conditions are described in Synthetic Organic Methodology: Comprehensive Organic Transformations. A Guide to Functional Group Preparations, Larock, R. C. 1989 p 981.
ii) activation of compound of formula IXb, wherein R₆ is as defined above, R is hydrogen and X is halogen such as chlorine, by methods known to those skilled in the art and described in, for example, Tetrahedron, 2005, 61 (46), 10827-10852, to form the compound IXa, wherein R₆ is as defined above and X is halogen such as chlorine and wherein X₀₀ is halogen, preferably chlorine. For example, compounds IXa where X₀₀ is halogen, preferably chlorine, are formed by treatment of compounds of formula IXb wherein R is H with, for example, oxalyl chloride, (COCl)₂, or thionyl chloride, SOCl₂, in the presence of catalytic quantities of N,N-dimethylformamide, DMF in inert solvents such as methylene chloride, CH₂Cl₂, or tetrahydrofuran, THF, at temperatures between 20° C. to 120° C. Alternatively, treatment of compounds of formula IXb with, for example, 1-ethyl-3-(3-dimethylaminopropyl) carbodiimide (EDC) or dicyclohexyl carbodiimide (DCC) will generate the compound IXa, wherein X₀₀ is X₀₁ or X₀₂ respectively, in an inert solvent, such as pyridine or tetrahydrofuran, THF, optionally in the presence of a base, such as triethylamine, at temperatures between 25° C. to 180° C.; followed by iii) treatment of the compound IXa, wherein R₁ₐ, R_{1b}, R₂, R₃, R₄, R₅ and R₆ are as defined in formula I above with a compound of formula VI, wherein R₁ₐ, R_{1b}, R₂, R₃, R₄ and R₅ are as defined in formula I above, optionally in the presence of a base, such as triethylamine or pyridine, in an inert solvent such as dichloromethane, tetrahydrofuran, dioxane or toluene, at temperatures between 0 and 80° C., to form the compounds of formula X.

Preparation of compounds of the formula IXc, IXa and IXb, wherein R₆ is as defined in formula I above, can be carried out by those skilled in the art following literature, for example, Tetrahedron Letters (2008), 49(14), 2286-2288; Bull. Soc. Chim. Fr. 1974, p 531; Synth. Commun. 2002, 32, 2821; Chemistry Select 2017 2(1), 356-363; Bioorganic & Medicinal Chemistry 2017 25(7), 2043-2056; Synthesis, 48(8), 1202-1216; 2016.

Compounds of the formula XI, wherein R₁ₐ, R_{1b}, R₂, R₃, R₄, R₅ and R₆ are as defined in formula I above and A⁻ is an anion such as for example AlCl₄⁻ or Cl⁻, may be prepared by reaction of compound of formula X in presence or not of a lewis catalysis such as aluminum chloride in a inert solvent such as 1,2-dichloroethane, at temperatures between 0 and 100° C.

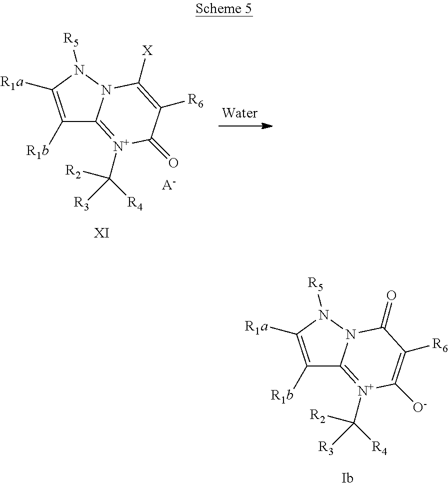

Compounds of formula Ib, wherein R₁ₐ, R_{1b}, R₂, R₃, R₄, R₅ and R are as defined in formula I above, can be prepared (as shown in scheme above) by reaction of compound of formula XI, wherein R₁ₐ, R_{1b}, R₂, R₃, R₄, R₅ and R are as defined above and X is halogen such as chlorine and A⁻ is an anion such as for example AlCl₄⁻ or Cl⁻, with water in presence, or not, of a inert solvent such as tetrahydrofuran at at temperatures between 20° C. to reflux of the used mixture of solvent. Alternatively, compounds of formula Ib, wherein R₁ₐ, R_{1b}, R₂, R₃, R₄, R₅ and R₆ are as defined in formula I above, can be obtained during the formation of compounds of formula XI if an aqueous work-up is used.

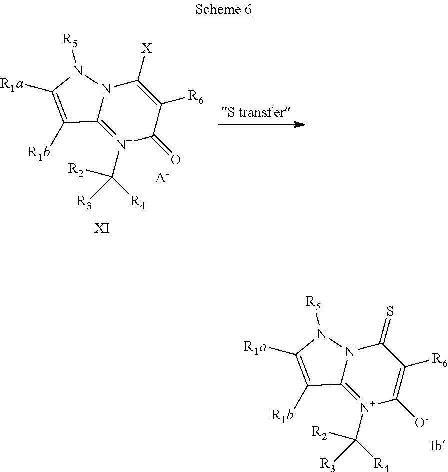

Compounds of formula Ib′, wherein R₁ₐ, R_{1b}, R₂, R₃, R₄, R₅ and R₆ are as defined in formula I above, can be prepared (as shown in scheme above) by reaction of compound of formula XI, wherein R₁ₐ, R_{1b}, R₂, R₃, R₄, R₅ and R₆ are as defined above and X is halogen such as chlorine and A⁻ is an anion such as for example AlCl₄⁻ or Cl⁻, with a compound able to deliver sulfur such as hydrated sodium sulfide, in presence, or not, of a inert solvent such as methanol, at temperatures between 0° C. to reflux and preferably at room temperature.

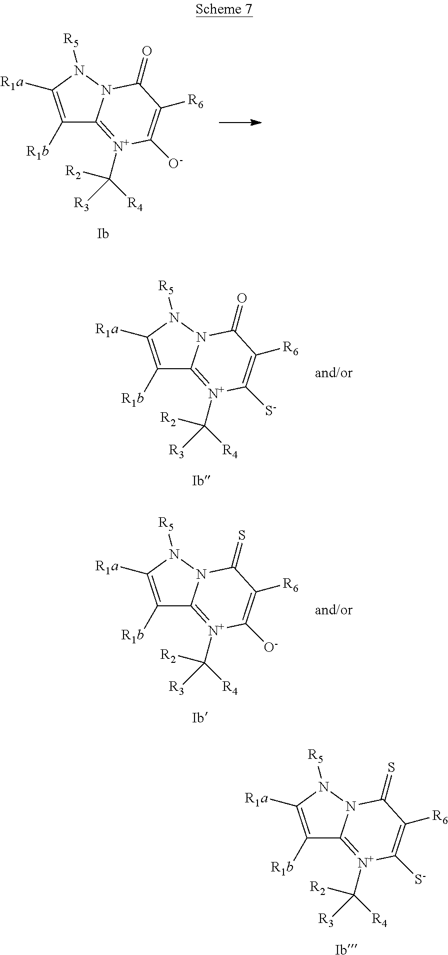

Alternatively, compounds of formula Ib′, Ib″ or/and Ib″′, wherein R₁ₐ, R_{1b}, R₂, R₃, R₄, R₅ and R₆ are as defined in formula I above, can be prepared (as shown in scheme above) by reaction of compound of formula Ib, wherein R₁ₐ, R_{1b}, R₂, R₃, R₄, R₅ and R₆ are as defined in formula I above, with a reagent that could transfer a sulphur atom such as, for example, the Lawesson's reagent in a solvent such as, for example dimethylformamide or toluene, usually at temperature between 25° C. to 150° C. This type of transformation is known to a person skilled in the art and are, for example, described in Synthesis (2003), (13), 1929-1958.

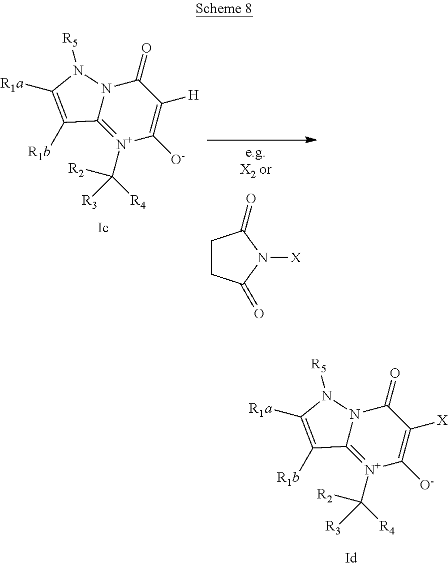

Compounds of formula Id, wherein R₁ₐ, R_{1b}, R₂, R₃, R₄ and R₅ are as defined in formula I above and X (corresponding to Re in formula I) is halogen can be prepared (as shown in scheme above) by halogenation, using, for example bromine or N-halosuccinimides. Typically, the reaction is performed in a inert solvent such as for example dichloromethane at temperatures between 0° C. to the boiling point of the reaction mixture.

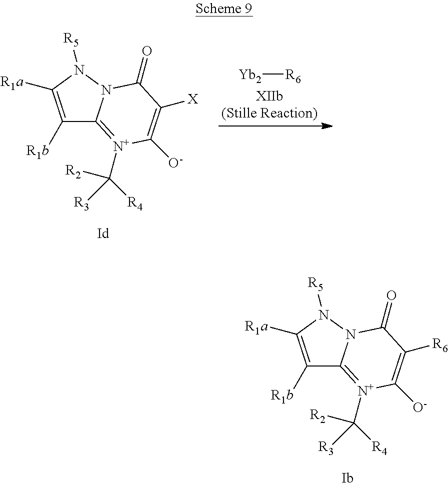

Compounds of formula Ib, wherein R₁ₐ, R_{1b}, R₂, R₃, R₄ and R₅ are as defined in formula I above and R₆ is, for example an aromatic or heteroaromatic, can be prepared (as shown in scheme above) by a Suzuki reaction, which involves for example, reacting compounds of formula Id, wherein X is a leaving group, for example, chlorine, bromine or iodine, or an aryl- or alkylsulfonate such as trifluoromethanesulfonate with compounds of formula XIIa, wherein Y_{b} can be a boron-derived functional group, as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction can be catalyzed by a palladium based catalyst, for example tetrakis(triphenylphosphine)-palladium or (1,1′bis(diphenylphosphino)-ferrocene)dichloropalladium-dichloromethane (1:1 complex), in presence of a base, like sodium carbonate or cesium fluoride, in a solvent or a solvent mixture, like, for example a mixture of 1,2-dimethoxyethane and water, or of dioxane and water, preferably under an inert atmosphere. The reaction temperature can preferentially range from room temperature to the boiling point of the reaction mixture. Such Suzuki reactions are well known to those skilled in the art and have been reviewed, for example Journal of Organometallic Chemistry (1999), 576(1-2), 147-168.

Alternatively, compounds of formula Ib can be prepared by a Stille reaction of compounds of formula XIIb wherein Yb2 is a trialkyl tin derivative, preferably tri-n-butyl tin, with compounds of formula Id. Such Stille reactions are usually carried out in the presence of a palladium catalyst, for example tetrakis(triphenylphosphine)palladium(0), or (1,1′bis(diphenylphosphino)-ferrocene)dichloropalladium-dichloromethane (1:1 complex), in an inert solvent such as DMF, acetonitrile, or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

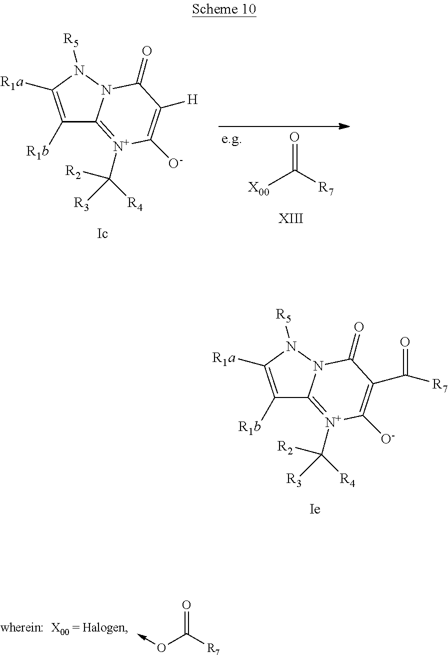

Compounds of formula Ie, wherein R₁ₐ, R_{1b}, R₂, R₃, R₄, R₅ and R₇ are as defined in formula I above, can be prepared (as shown in scheme above) by reaction of a compounds of formula Ic, wherein R₁ₐ, R_{1b}, R₂, R₃, R₄ and R₅ are as defined in formula I above and a compound of formula XIII, wherein R₇ is as defined above, and wherein X₀₀ is halogen or R₇C(O)O to form an anhydride in the presence or not of a lewis acid catalyst such as, for example, aluminium trichloride in presence of a solvent such as dichloromethane at temperatures between 0° C. to 100° C., preferably 25° C. These reaction are “Friedel-Crafts acylation” type reaction and methods are known to those skilled in the art and described in, for example, in March's Advanced Organic Chemistry: Reactions, Mechanisms, and Structure, 5th Edition p 712-714.

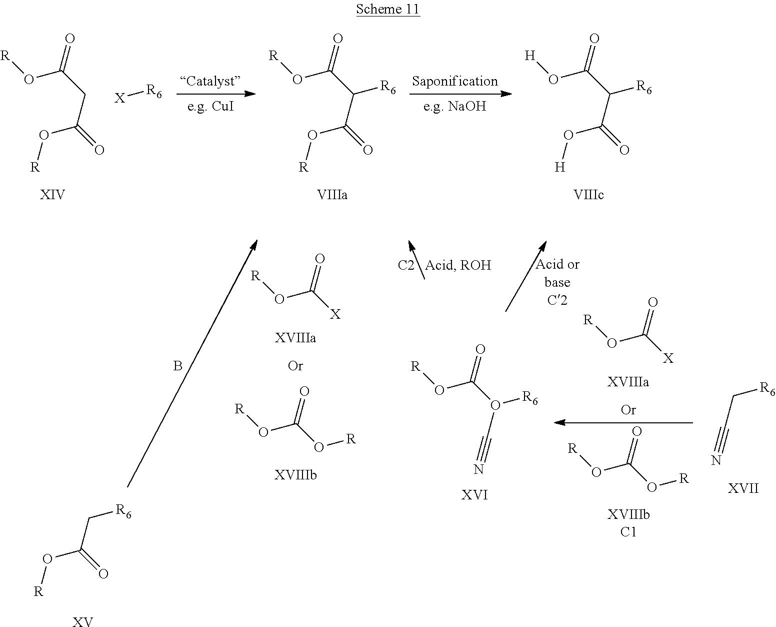

Compounds of formula VIIIa are commercially available or the preparation of compounds of formula VIIIa, wherein R is, for example C₁-C₄alkyl or VIIIc are very well known to those skilled in the art, for example:

A: Compounds of formula VIIIa can be prepared by coupling a compound of formula XIV (commercially available or easily prepared to those skilled in the art) via a catalysed coupling such as copper catalyst, for example copper(I) iodide or palladiumcatalyst such as Bis(dibenzylideneacetone) palladium in the presence of a base, such as potassium carbonate K₂CO₃ or cesium carbonate Cs₂CO₃, with or without an additive such as Adamantyl-di-tert-butylphosphine or L-proline, N,N′-dimethylcyclohexane-1,2-diamine or N,N′-dimethylethylene-diamine, in an inert solvent such as N-methylpyrrolidone NMP, toluene or dioxane at temperatures between 30-150° C., optionally under microwave irradiation. For examples, (“Cu” catalyst); Angewandte Chemie, International Edition 2012, 51(4), 1028-1032, S1028/1-S1028/80; Organic Letters 2007, 9(17), 3469-3472 (photochemistry); journal of the American Chemical Society 1980, 102(26), 7765-74 (“Pd” catalyst). Tetrahedron Letters 2015, 56(23), 3447-3450; Tetrahedron Letters 43 (2002) 2847-2849; Journal of Organic Chemistry 2002, 67(2), 541-555; This reaction working well when R₆ is aromatic or heteroaromatic. In other case, e.g. R₆ is alkyl, alkynyl or alkenyl, compounds of formula VIIIa can be prepared by reaction a compound of formula XIV (commercially available or easily prepared to those skilled in the art) via substitution of a halogen in presence of a base, such as sodium hydride or sodium ethanolate in an inert solvent such as benzene or ethanol at temperatures between 0° C.-150° C. For examples, Bioorganic & Medicinal Chemistry Letters 2008, 18(24), 6568-6572; Journal of Organic Chemistry 2014, 79(3), 1399-1405; Journal of Organic Chemistry 2002, 67(7), 2257-2262; Tetrahedron Letters 2003, 44(3), 503-506.
B: Compounds of formula VIIIa can be prepared via reaction of compounds of formula XV with compounds of formula (XVIIIa) or (XVIIIb) wherein X is halogen such as chlorine or R is aryl or alkyl such as phenyl, 1,3,5-trichlorophenyl or methyl in presence of a base, such as sodium hydride or HMDSLi in a inert solvent such as toluene or tetrahydrofuran. The reaction is carried out at a temperature of from −100° C. to +130° C., preferably from −78° C. to 100° C. For examples, WO 2016055431, Chemistry—A European Journal 2016, 22(2), 610-625; WO 2011017351.
C₁/C₂/C′2: Compounds of formula VIIIa, wherein R₆ is as defined in formula I above may be prepared by:
i) reaction of compounds of formula XVII with a compound of formula XVIIIa wherein X is a halogen, such as chlorine and or with a compound of formula XVIIIb wherein R is a aryl or C₁-C₄ alkyl group, such as methyl, phenyl or 1,3,5-trichlorophenyl to form compounds of formula XVI. The reaction is carried out in an inert solvent such as toluene or tetrahydrofuran in presence of a base such as sodium hydride or butyl lithium at a temperature of from −100° C. to +130° C., preferably from −78° C. to 100° C. These reactions are known to those skilled in the art, for example, European Journal of Organic Chemistry 2016, 2016(1), 210-227; Chemical Communications (Cambridge, United Kingdom), 2008, (21), 2474-2476. Compounds of formula XVII are commercially available or readily prepared by methods known in the art.
ii) hydrolysis of the nitrile group of compounds of formula XVI in presence of acid such as hydrochloric acid or a presence of a base such as sodium hydroxide in a solvent such as water or acetic acid at a temperature of from −20° C. to +130° C., give compounds of formula VIIIc via synthetic way C′2. This transformation is well known and conditions are described in Synthetic Organic Methodology: Comprehensive Organic Transformations. A Guide to Functional Group Preparations, Larock, R. C. 1989 p 993. Alliteratively, hydrolysis of the nitrile group of compounds of formula XVI in presence of acid such as hydrochloric acid or sulfuric in a solvent such as alcohol, such as methanol, in presence or not of water at a temperature of from −20° C. to +130° C., give compounds of formula VIIIa via synthetic way C₂. This transformation is well known and conditions are described in Synthetic Organic Methodology: Comprehensive Organic Transformations. A Guide to Functional Group Preparations, Larock, R. C. 1989 p 993.
D: Compound of formula (VIIIc), wherein R₆ as defined in formula I above, may be prepared by reaction of a compound of formula (VIIIa), wherein R is C₁-C₄ alkyl via hydrolysis. For instance, in the case where R is methyl or ethyl, the hydrolysis can be done with water and a base, such as potassium hydroxide or lithium hydroxide, in the absence or in the presence of a solvent, such as, for instance, tetrahydrofuran or methanol. In the case where R is, for example, tert-butoxy, the hydrolysis is done in the presence of acid, such as trifluoroacetic acid or hydrochloric acid. The reaction is carried out at a temperature of from −120° C. to +130° C., preferably from −100° C. to 100° C. For examples, as described in Biochemistry 2000, 39(15), 4543-4551; WO 2011017351 or WO 2009099929.

Alternatively, the synthetic way described in scheme 4 and 5 could be applied to other scaffolds such as for example, amino pyridine as described in scheme 12 and scheme 12b. These scaffolds were described in, for example, WO11017342, WO16171053 or WO09099929. For example, WO11017342 or WO09099929 contained synthesis of Intermediate XIX and alternative synthesis to compounds of formula XXIII via a more classical synthesis as described in scheme 3.

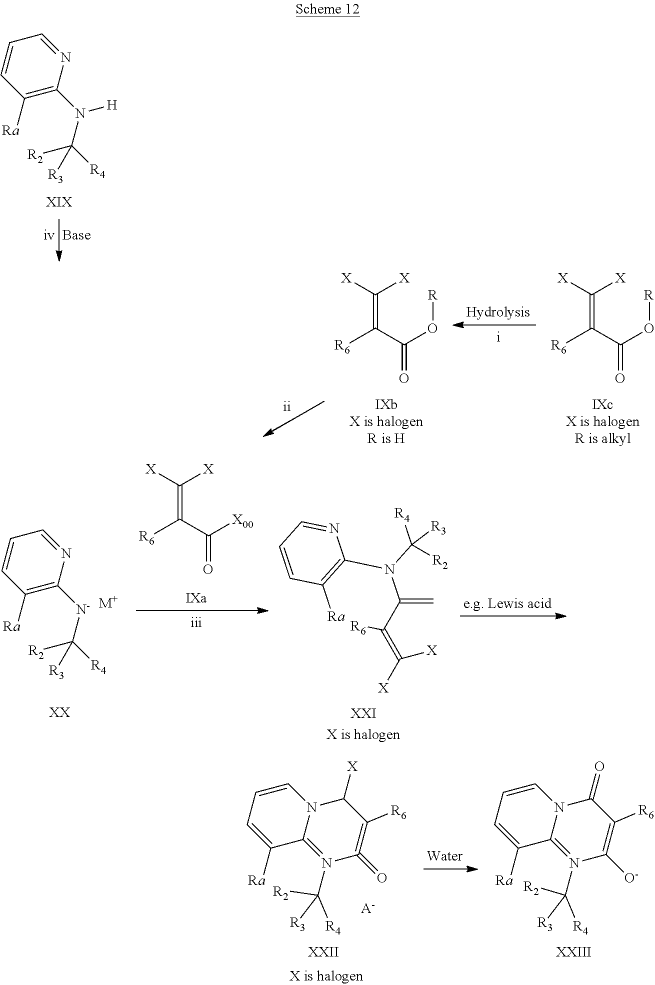

Compounds of the formula XXIII, wherein R₂, R₃, R₄ and Re are as defined in formula I above and Ra is hydrogen or methyl, may be prepared by:

step iv): Compound of formula XX, wherein R₂, R₃ and R₄ are as defined in formula I above and Ra is, for example, hydrogen or methyl, may be prepared by reaction of a compound of formula XIX, wherein R₂, R₃ and R₄ are as defined in formula I above and Ra is, for example, hydrogen or methyl via reaction with a base such as a isopropyl magnesium chloride or lithiumdiisopropylamid, in the presence of a inert solvent or a mixture of solvent such as tetrahydrofuran or toluene, at temperatures between −100° C. to +130° C., preferably from −78° C. to 80° C.
step iii): compounds of formula XXI, wherein R₂, R₃, R₄ and R₆ are as defined in formula I above and Ra is, for example, hydrogen or methyl, may be prepared by treatment of the compound IXa, prepared as described in scheme 4 (step I, ii) with a compound of formula XX, wherein R₂, R₃ and R₄ are as defined in formula I above and Ra is, for example, hydrogen or methyl, in an inert solvents such as tetrahydrofuran or toluene, at temperatures between −20° C. and 80° C.

Compounds of the formula XXII, wherein R₂, R₃, R₄ and R₆ are as defined in formula I above and Ra is, for example, hydrogen or methyl and A⁻ is an anion such as for example AlCl₄⁻ or Cl⁻, may be prepared by reaction of compound of formula XXI in presence or not of a Lewis acid catalysis such as aluminum chloride in a inert solvent such as 1,2-dichloroethane, at temperatures between 0° C. and 120° C.

Compounds of the formula XXII, wherein R₂, R₃, R₄ and R₆ are as defined in formula I above and Ra is, for example, hydrogen or methyl, and A⁻ is an anion such as for example AlCl₄⁻ or Cl⁻ are reactive intermediates and could react with water, in presence or not of a inert solvent such a tetrahydrofuran or 1,2-dichloroethane, to form compounds of the formula XXIII, wherein R₂, R₃, R₄ and R₆ are as defined in formula I above and Ra is, for example, hydrogen or methyl.

The formation of compounds of formula XXIII could be done on isolated compounds of formula XXII or directly via hydrolysis with water during the work up following the formation of compounds of formula XXII.

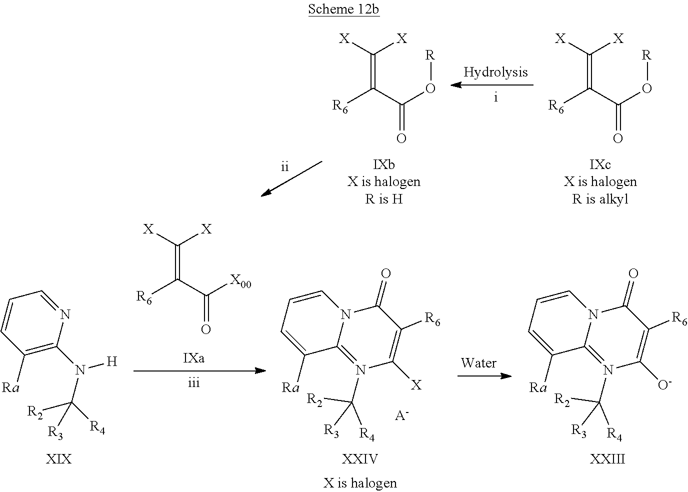

Alternatively, compounds of the formula XXIV, wherein R₂, R₃, R₄ and R₆ are as defined in formula I above and Ra is, for example, hydrogen or methyl and A⁻ is an anion such as for example Cl⁻, may be prepared by reaction of a compound of formula XIX wherein R₂, R₃ and R₄ are as defined in formula I above and Ra is, for example, hydrogen or methyl with a compound of formula IXa (prepared as described in scheme 3) in the presence of a base, bases employed in excess or not, such as Huenig's base in solvent or mixture of solvents such as 1,2-dichloroethane or tetrahydrofuran.

Compounds of the formula XXIV, wherein R₂, R₃, R₄ and R₆ are as defined in formula I above and Ra is, for example, hydrogen or methyl and A⁻ is an anion such as for example Cl⁻ are reactive intermediates and could react with water, in presence or not of a inert solvent such a tetrahydrofuran or 1,2-dichloroethane, to form compounds of the formula XXIII, wherein R₂, R₃, R₄ and R₆ are as defined in formula I above and Ra is, for example, hydrogen or methyl. Compounds of the formula XXIV, wherein R₂, R₃, R₄ and R₆ are as defined in formula I above and Ra is, for example, hydrogen or methyl may be isolated or react directly with water, during the work up after them formation. Compounds of formula XXIII are also examples of active ingredients having pesticidal activity.

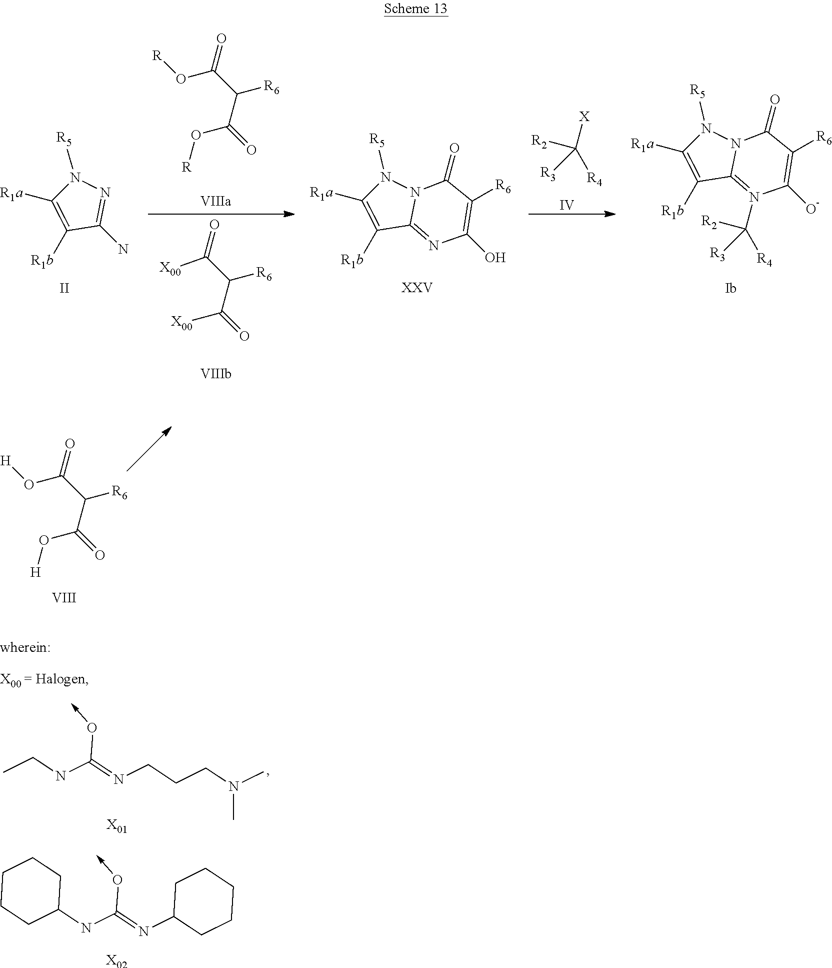

Compounds of the formula XXV, wherein R₁ₐ, R_{1b}, R₅ and R₆ are as defined in formula I above and may be prepared by reaction with compounds of formula II and compounds of formula VIIIa or VIIIb as described in scheme 1 using the same conditions described for the scheme 1. Then, further reaction of compounds of the formula XXV with Compounds of the formula IV using the same conditions described in scheme 1 give Compounds of the formula I wherein R₁ₐ, R_{1b}, R₂, R₃, R₅ and R₆ are as defined in formula I above.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an) other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties, can be obtained in free form or in the form of salts.

The compounds of formula I and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

Certain intermediates used in the process are also novel.

Accordingly, in a further aspect the present invention provides a compound of formulae IXa, IXb and IXc

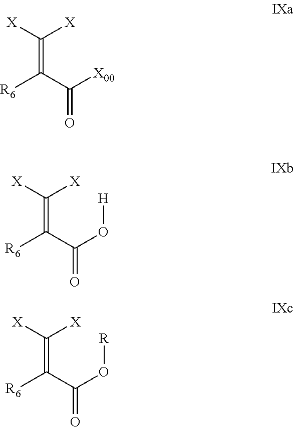

where R₆ in each of IXa, IXb and IXc is 3,5-dichloro phenyl, or 3-trifluormethylphenyl; X in each of IXa, IXb and IXc is a halogen atom (preferably chlorine; R in formula IXc is methyl, or ethyl, and X₀₀ is a halogen atom, or an iso-urea-containing compound, such as 1,3-dicyclohexyl-isourea-2-yl; and acceptable salts, stereoisomers, enantiomers, tautomers and N-oxides.

In a further aspect the present invention provides a compound of formula X

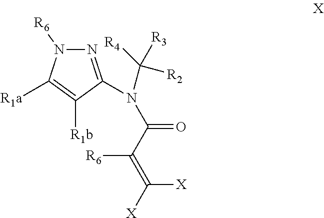

where R₁ₐ, R_{1b}, R₂, R₃, R₄, R₅ and R₆ are as defined in formula I in the first aspect and X is a halogen (preferably Cl); and acceptable salts, stereoisomers, enantiomers, tautomers and N-oxides.

In a further aspect the present invention provides a compound of formula XXI

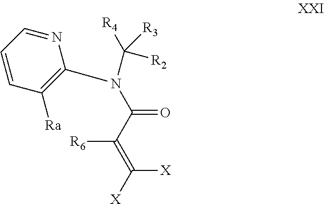

R₂, R₃, R₄, and R₆ are as defined in formula I in the first aspect, Ra is hydrogen or methyl, and X is a halogen (preferably Cl); and acceptable salts, stereoisomers, enantiomers, tautomers and N-oxides. In an embodiment of formula XXI, R₂ and R₃ are each H, R₄ is 2-chloro-1,3-thiazol-5-yl or pyrimidin-5-yl and R₆ is 3,5-dichloro phenyl, or 3-trifluormethylphenyl.

In a further aspect the present invention provides a compound of formula XI

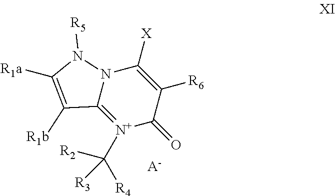

where R₁ₐ, R_{1b}, R₂, R₃, R₄, R₅ and R₆ are as defined in formula I in the first aspect, X is a halogen (preferably Cl), and A⁻ is an anion, preferably selected from AlCl₄⁻ and Cl⁻; and acceptable salts, stereoisomers, enantiomers, tautomers and N-oxides.

In a further aspect the present invention provides a compound of formulae XXII and XXIV

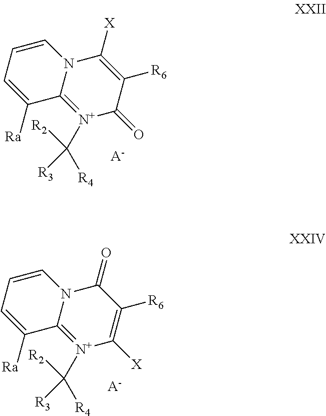

where R₂, R₃, R₄, and R₆ are, independent of formula XXII and XXIV, as defined in formula I in the first aspect, Ra is hydrogen or methyl, X is a halogen (preferably Cl), and A⁻ is an anion, preferably selected from AlCl₄⁻ and Cl⁻; and acceptable salts, stereoisomers, enantiomers, tautomers and N-oxides. In an embodiment of formulae XXII and XXIV, R₂ and R₃ are each H, R₄ is 2-chloro-1,3-thiazol-5-yl or pyrimidin-5-yl and R₆ is 3,5-dichloro phenyl, or 3-trifluormethylphenyl.

For the avoidance of doubt, the embodiments for the various substituents provided above is also applicable to the intermediate compounds (different aspects of the invention), wherever the corresponding substituent appears.

In a further aspect, the present invention makes available a process for preparing a compound of formula Ib, wherein R₁ₐ, R_{1b}, R₂, R₃, R₄, R₅ and R₆ are as defined in formula I above, by
(i) reaction of compound VI (where R₁ₐ, R_{1b}, R₂, R₃, R₄, and R₅ are as defined in formula I) with a compound of formula VIIIa wherein R is aryl or alkyl;

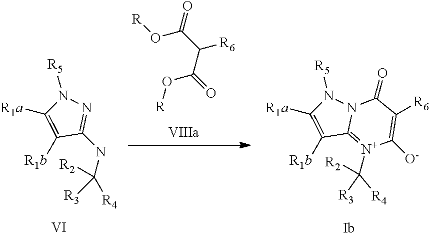

or
(ii) reaction of compound of formula XI, wherein R₁ₐ, R_{1b}, R₂, R₃, R₄, R₅ and R₆ are as defined above for formula I and X is halogen, such as chlorine, and A⁻ is an anion, such as for example AlCl₄⁻ or Cl⁻;

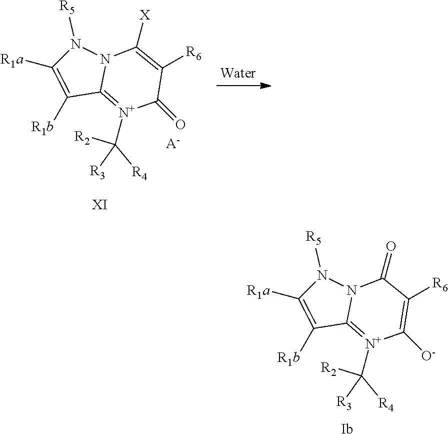

or
(iii) reacting compounds of formula Id (wherein wherein R₁ₐ, R_{1b}, R₂, R₃, R₄, R₅ and R₆ are as defined for formula I above and X is a leaving group) with compounds of formula XIIa, wherein Y_{b1} can be a boron-derived functional group; or reacting compounds of formula Id, wherein X is a leaving group with compounds of formula XIIb, wherein Yb2 is a trialkyl tin derivative.

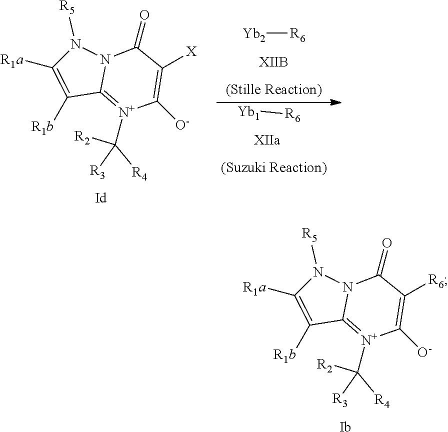

The compounds according to the following Tables 1 and 2 below can be prepared according to the methods described herein. The compounds which follow are intended to illustrate the invention and show particularly preferred compounds of formulae I, and IXa respectively. “Ph” represents the phenyl group.

Table 1: This table discloses the 136 compounds of the formula Ia, where Ph is phenyl:

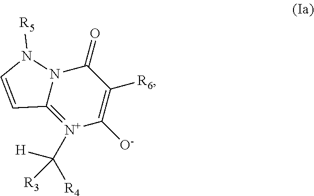

TABLE 1

| Comp. | | | | |
|---|---|---|---|---|
| No. | R₂ | R₅ | R₄ | R₆ |
| 1.001 | H | CH₃ | 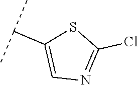 | 3,5-diCl—Ph |
| 1.002 | H | CH₃ | 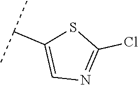 | m-BrPh |
| 1.003 | H | CH₃ | 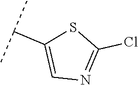 | p-BrPh |
| 1.004 | H | CH₃ | 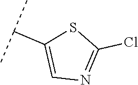 | 3,5-diBr—Ph |
| 1.005 | H | CH₃ | 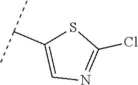 | 3-Cl, 5-Br—Ph |
| 1.006 | H | CH₃ | 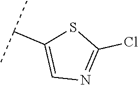 | 3-Cl, 5-CF₃—Ph |
| 1.007 | H | CH₃ | 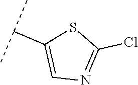 | CF₃ |
| 1.008 | H | CH₃ | 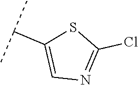 | —C(O)Ph |
| 1.009 | H | CH₃ | 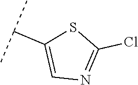 | —C(O) 3,5-diCl—Ph |
| 1.010 | H | CH₃ | 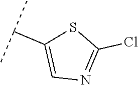 | —CH₂Ph |
| 1.011 | H | CH₃ | 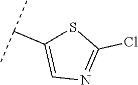 | m-ClPh |
| 1.012 | H | CH₃ | 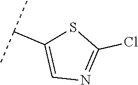 | naphthyl |
| 1.013 | H | CH₃ | 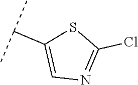 | 2-Br-naphth-6-yl |
| 1.014 | H | CH₃ | 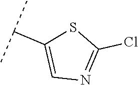 | m-CF₃Ph |
| 1.015 | H | CH₃ | 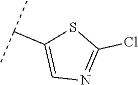 | 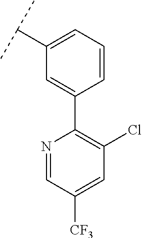 |
| 1.016 | H | CH₃ | 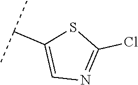 | 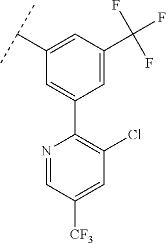 |
| 1.017 | H | CH₃ | 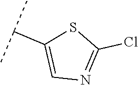 | 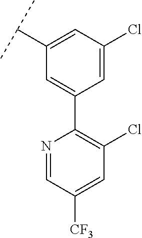 |
| 1.018 | H | CH₂CH₃ | 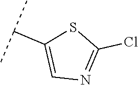 | 3,5-diCl—Ph |
| 1.019 | H | CH₂CH₃ | 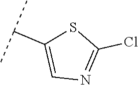 | m-BrPh |
| 1.020 | H | CH₂CH₃ | 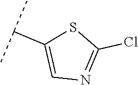 | p-BrPh |
| 1.021 | H | CH₂CH₃ | 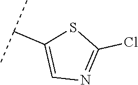 | 3,5-diBr—Ph |
| 1.022 | H | CH₂CH₃ | 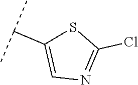 | 3-Cl, 5-Br—Ph |
| 1.023 | H | CH₂CH₃ | 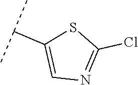 | 3-Cl, 5-CF₃—Ph |
| 1.024 | H | CH₂CH₃ | 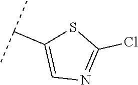 | CF₃ |
| 1.025 | H | CH₂CH₃ | 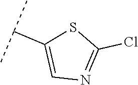 | —C(O)Ph |
| 1.026 | H | CH₂CH₃ | 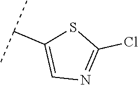 | —C(O) 3,5-diCl—Ph |
| 1.027 | H | CH₂CH₃ | 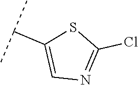 | —CH₂Ph |
| 1.028 | H | CH₂CH₃ | 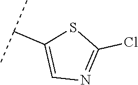 | m-ClPh |
| 1.029 | H | CH₂CH₃ | 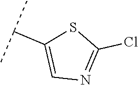 | naphthyl |
| 1.030 | H | CH₂CH₃ | 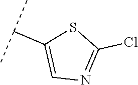 | 2-Br-naphth-6-yl |
| 1.031 | H | CH₂CH₃ | 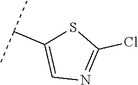 | m-CF₃Ph |
| 1.032 | H | CH₂CH₃ | 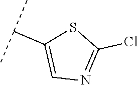 | 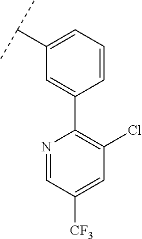 |
| 1.033 | H | CH₂CH₃ | 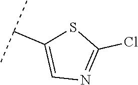 | 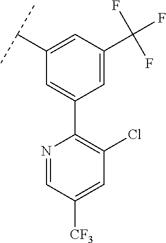 |
| 1.034 | H | CH₂CH₃ | 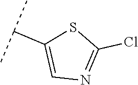 | 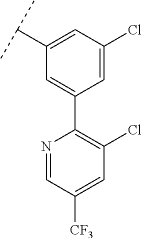 |
| 1.035 | CH₂CN | CH₃ | H | 3,5-diCl—Ph |
| 1.036 | CH₂CN | CH₃ | H | m-BrPh |
| 1.037 | CH₂CN | CH₃ | H | p-BrPh |
| 1.038 | CH₂CN | CH₃ | H | 3,5-diBr—Ph |
| 1.039 | CH₂CN | CH₃ | H | 3-Cl, 5-Br—Ph |
| 1.040 | CH₂CN | CH₃ | H | 3-Cl, 5-CF₃—Ph |
| 1.041 | CH₂CN | CH₃ | H | CF₃ |
| 1.042 | CH₂CN | CH₃ | H | —C(O)Ph |
| 1.043 | CH₂CN | CH₃ | H | —C(O) 3,5-diCl—Ph |
| 1.044 | CH₂CN | CH₃ | H | —CH₂Ph |
| 1.045 | CH₂CN | CH₃ | H | m-ClPh |
| 1.046 | CH₂CN | CH₃ | H | naphthyl |
| 1.047 | CH₂CN | CH₃ | H | 2-Br-naphth-6-yl |
| 1.048 | CH₂CN | CH₃ | H | m-CF₃Ph |
| 1.049 | CH₂CN | CH₃ | H | 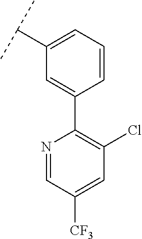 |
| 1.050 | CH₂CN | CH₃ | H | 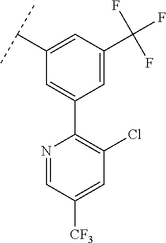 |
| 1.051 | CH₂CN | CH₃ | H | 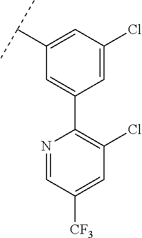 |
| 1.052 | CH₂CN | CH₂CH₃ | H | 3,5-diCl—Ph |
| 1.053 | CH₂CN | CH₂CH₃ | H | m-BrPh |
| 1.054 | CH₂CN | CH₂CH₃ | H | p-BrPh |
| 1.055 | CH₂CN | CH₂CH₃ | H | 3,5-diBr—Ph |
| 1.056 | CH₂CN | CH₂CH₃ | H | 3-Cl, 5-Br—Ph |
| 1.057 | CH₂CN | CH₂CH₃ | H | 3-Cl, 5-CF₃—Ph |
| 1.058 | CH₂CN | CH₂CH₃ | H | CF₃ |
| 1.059 | CH₂CN | CH₂CH₃ | H | —C(O)Ph |
| 1.060 | CH₂CN | CH₂CH₃ | H | —C(O) 3,5-diCl—Ph |
| 1.061 | CH₂CN | CH₂CH₃ | H | —CH₂Ph |
| 1.062 | CH₂CN | CH₂CH₃ | H | m-ClPh |
| 1.063 | CH₂CN | CH₂CH₃ | H | naphthyl |
| 1.064 | CH₂CN | CH₂CH₃ | H | 2-Br-naphth-6-yl |
| 1.065 | CH₂CN | CH₂CH₃ | H | m-CF₃Ph |
| 1.066 | CH₂CN | CH₂CH₃ | H | 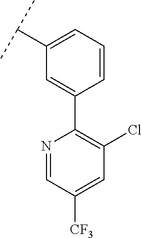 |
| 1.067 | CH₂CN | CH₂CH₃ | H | 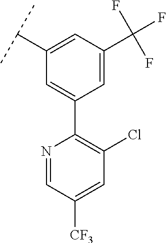 |
| 1.068 | CH₂CN | CH₂CH₃ | H | 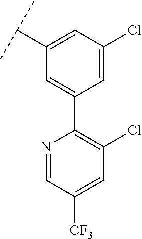 |
| 1.069 | H | CH₃ | 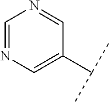 | 3,5-diCl—Ph |
| 1.070 | H | CH₃ | 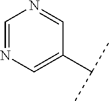 | m-BrPh |
| 1.071 | H | CH₃ | 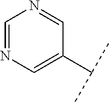 | p-BrPh |
| 1.072 | H | CH₃ | 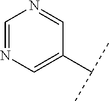 | 3,5-diBr—Ph |
| 1.073 | H | CH₃ | 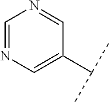 | 3-Cl, 5-Br—Ph |
| 1.074 | H | CH₃ | 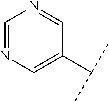 | 3-Cl, 5-CF₃—Ph |
| 1.075 | H | CH₃ | 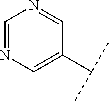 | CF₃ |
| 1.076 | H | CH₃ | 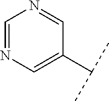 | —C(O)Ph |
| 1.077 | H | CH₃ | 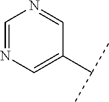 | —C(O) 3,5-diCl—Ph |
| 1.078 | H | CH₃ | 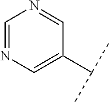 | —CH₂Ph |
| 1.079 | H | CH₃ | 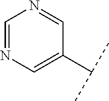 | m-ClPh |
| 1.080 | H | CH₃ | 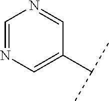 | naphthyl |
| 1.081 | H | CH₃ | 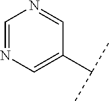 | 2-Br-naphth-6-yl |
| 1.082 | H | CH₃ | 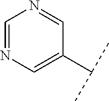 | m-CF₃Ph |
| 1.083 | H | CH₃ | 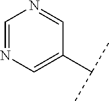 | 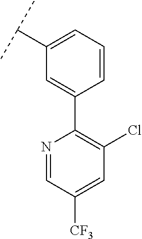 |
| 1.084 | H | CH₃ | 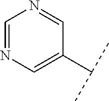 | 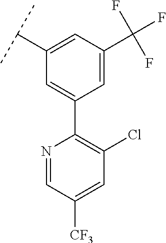 |
| 1.085 | H | CH₃ | 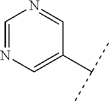 | 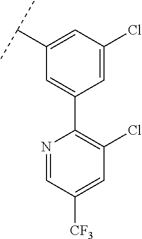 |
| 1.086 | H | CH₂CH₃ | 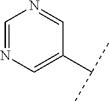 | 3,5-diCl—Ph |
| 1.087 | H | CH₂CH₃ | 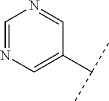 | m-BrPh |
| 1.088 | H | CH₂CH₃ | 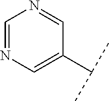 | p-BrPh |
| 1.089 | H | CH₂CH₃ | 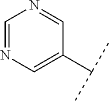 | 3,5-diBr—Ph |
| 1.090 | H | CH₂CH₃ | 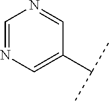 | 3-Cl, 5-Br—Ph |
| 1.091 | H | CH₂CH₃ | 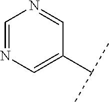 | 3-Cl, 5-CF₃—Ph |
| 1.092 | H | CH₂CH₃ | 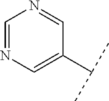 | CF₃ |
| 1.093 | H | CH₂CH₃ | 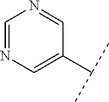 | —C(O)Ph |
| 1.094 | H | CH₂CH₃ | 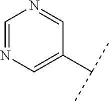 | —C(O) 3,5-diCl—Ph |
| 1.095 | H | CH₂CH₃ | 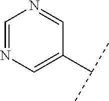 | —CH₂Ph |
| 1.096 | H | CH₂CH₃ | 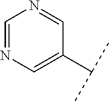 | m-ClPh |
| 1.097 | H | CH₂CH₃ | 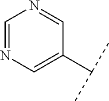 | naphthyl |
| 1.098 | H | CH₂CH₃ | 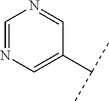 | 2-Br-naphth-6-yl |
| 1.099 | H | CH₂CH₃ | 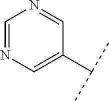 | m-CF₃Ph |
| 1.100 | H | CH₂CH₃ | 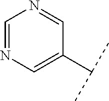 | 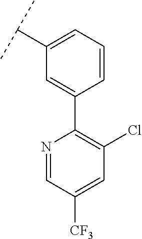 |
| 1.101 | H | CH₂CH₃ | 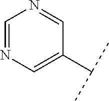 | 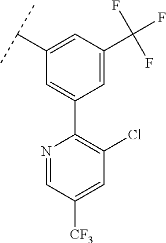 |
| 1.102 | H | CH₂CH₃ | 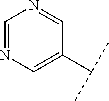 | 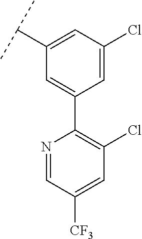 |
| 1.103 | H | CH₃ | 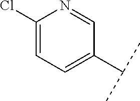 | 3,5-diCl—Ph |
| 1.104 | H | CH₃ | 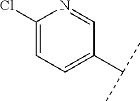 | m-BrPh |
| 1.105 | H | CH₃ | 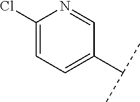 | p-BrPh |
| 1.106 | H | CH₃ | 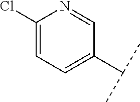 | 3,5-diBr—Ph |
| 1.107 | H | CH₃ | 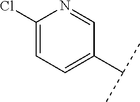 | 3-Cl, 5-Br—Ph |
| 1.108 | H | CH₃ | 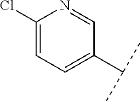 | 3-Cl, 5-CF₃—Ph |
| 1.109 | H | CH₃ | 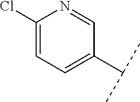 | CF₃ |
| 1.110 | H | CH₃ | 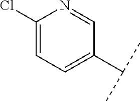 | —C(O)Ph |
| 1.111 | H | CH₃ | 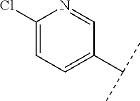 | —C(O) 3,5-diCl—Ph |
| 1.112 | H | CH₃ | 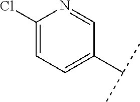 | —CH₂Ph |
| 1.113 | H | CH₃ | 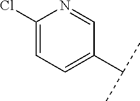 | m-ClPh |
| 1.114 | H | CH₃ | 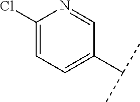 | naphthyl |
| 1.115 | H | CH₃ | 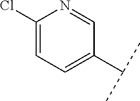 | 2-Br-naphth-6-yl |
| 1.116 | H | CH₃ | 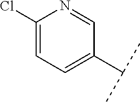 | m-CF₃Ph |
| 1.117 | H | CH₃ | 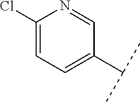 | 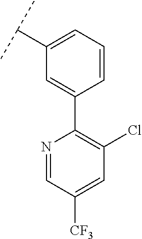 |
| 1.118 | H | CH₃ | 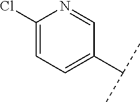 | 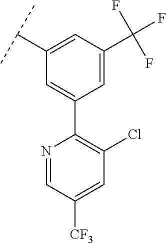 |
| 1.119 | H | CH₃ | 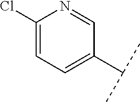 | 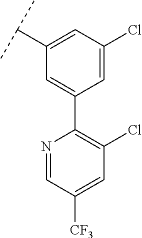 |
| 1.120 | H | CH₂CH₃ | 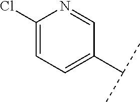 | 3,5-diCl—Ph |
| 1.121 | H | CH₂CH₃ | 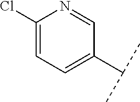 | m-BrPh |
| 1.122 | H | CH₂CH₃ | 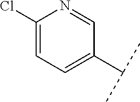 | p-BrPh |
| 1.123 | H | CH₂CH₃ | 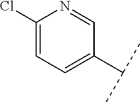 | 3,5-diBr—Ph |
| 1.124 | H | CH₂CH₃ | 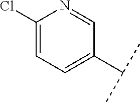 | 3-Cl, 5-Br—Ph |
| 1.125 | H | CH₂CH₃ | 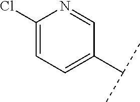 | 3-Cl, 5-CF₃—Ph |
| 1.126 | H | CH₂CH₃ | 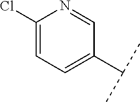 | CF₃ |
| 1.127 | H | CH₂CH₃ | 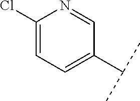 | —C(O)Ph |
| 1.128 | H | CH₂CH₃ | 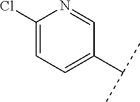 | —C(O) 3,5-diCl—Ph |
| 1.129 | H | CH₂CH₃ | 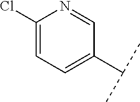 | —CH₂Ph |
| 1.130 | H | CH₂CH₃ | 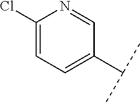 | m-ClPh |
| 1.131 | H | CH₂CH₃ | 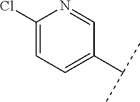 | naphthyl |
| 1.132 | H | CH₂CH₃ | 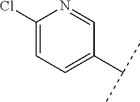 | 2-Br-naphth-6-yl |
| 1.133 | H | CH₂CH₃ | 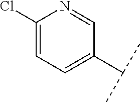 | m-CF₃Ph |
| 1.134 | H | CH₂CH₃ | 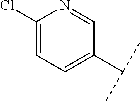 | 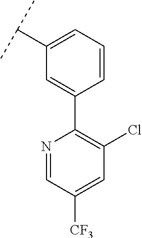 |
| 1.135 | H | CH₂CH₃ | 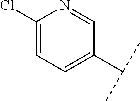 | 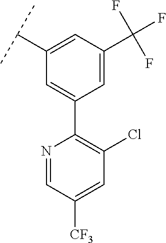 |
| 1.136 | H | CH₂CH₃ | 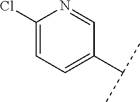 | 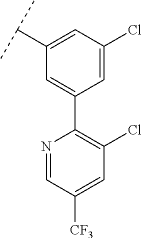 |

and the N-oxides of the compounds of Table 1.

Also made available are 136 compounds of formula X, wherein R₁ₐ, R_{1b}, R₃ are each hydrogen, X is a halogen (such as Cl) and A⁻ is an anion (such as AlCl₄⁻), and R₂, R₄, R₅ and R₆ are as defined in formula Ia in Table 1.

Table 2: This table discloses the 131 compounds of intermediates of formula IX:

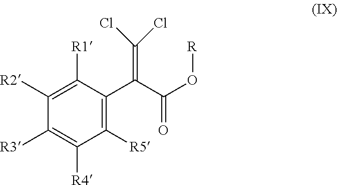

TABLE 2

| Comp. No. | R₁^{′} | R₂^{′} | R₃^{′} | R₄^{′} | R₅^{′} | R |
|---|---|---|---|---|---|---|
| 2.001 | H | Cl | H | Cl | H | H |
| 2.002 | H | Cl | H | Cl | H | CH₃ |
| 2.003 | H | Cl | H | Cl | H | CH₂CH₃ |
| 2.004 | H | Br | H | Cl | H | H |
| 2.005 | H | Br | H | Cl | H | CH₃ |
| 2.006 | H | Br | H | Cl | H | CH₂CH₃ |
| 2.007 | H | I | H | Cl | H | H |
| 2.008 | H | I | H | Cl | H | CH₃ |
| 2.009 | H | I | H | Cl | H | CH₂CH₃ |
| 2.010 | H | Cl | H | H | H | H |
| 2.011 | H | Cl | H | H | H | CH₃ |
| 2.012 | H | Cl | H | H | H | CH₂CH₃ |
| 2.013 | H | Br | H | H | H | H |
| 2.014 | H | Br | H | H | H | CH₃ |
| 2.015 | H | Br | H | H | H | CH₂CH₃ |
| 2.016 | H | I | H | H | H | H |
| 2.017 | H | I | H | H | H | CH₃ |
| 2.018 | H | I | H | H | H | CH₂CH₃ |
| 2.019 | H | Cl | H | CF₃ | H | H |
| 2.020 | H | Cl | H | CF₃ | H | CH₃ |
| 2.021 | H | Cl | H | CF₃ | H | CH₂CH₃ |
| 2.022 | H | Br | H | CF₃ | H | H |
| 2.023 | H | Br | H | CF₃ | H | CH₃ |
| 2.024 | H | Br | H | CF₃ | H | CH₂CH₃ |
| 2.025 | H | I | H | CF₃ | H | H |
| 2.026 | H | I | H | CF₃ | H | CH₃ |
| 2.027 | H | I | H | CF₃ | H | CH₂CH₃ |
| 2.028 | H | H | H | CF₃ | H | H |
| 2.029 | H | H | H | CF₃ | H | CH₃ |
| 2.030 | H | H | H | CF₃ | H | CH₂CH₃ |
| 2.031 | H | Cl | H | CF₂CF₃ | H | H |
| 2.032 | H | Cl | H | CF₂CF₃ | H | CH₃ |
| 2.033 | H | Cl | H | CF₂CF₃ | H | CH₂CH₃ |
| 2.034 | H | Br | H | CF₂CF₃ | H | H |
| 2.035 | H | Br | H | CF₂CF₃ | H | CH₃ |
| 2.036 | H | Br | H | CF₂CF₃ | H | CH₂CH₃ |
| 2.037 | H | I | H | CF₂CF₃ | H | H |
| 2.038 | H | I | H | CF₂CF₃ | H | CH₃ |
| 2.039 | H | I | H | CF₂CF₃ | H | CH₂CH₃ |
| 2.040 | H | H | H | CF₂CF₃ | H | H |
| 2.041 | H | H | H | CF₂CF₃ | H | CH₃ |
| 2.042 | H | H | H | CF₂CF₃ | H | CH₂CH₃ |
| 2.043 | H | Cl | H | OCF₃ | H | H |
| 2.044 | H | Cl | H | OCF₃ | H | CH₃ |
| 2.045 | H | Cl | H | OCF₃ | H | CH₂CH₃ |
| 2.046 | H | Br | H | OCF₃ | H | H |
| 2.047 | H | Br | H | OCF₃ | H | CH₃ |
| 2.048 | H | Br | H | OCF₃ | H | CH₂CH₃ |
| 2.049 | H | I | H | OCF₃ | H | H |
| 2.050 | H | I | H | OCF₃ | H | CH₃ |
| 2.051 | H | I | H | OCF₃ | H | CH₂CH₃ |
| 2.052 | H | H | H | OCF₃ | H | H |
| 2.053 | H | H | H | OCF₃ | H | CH₃ |
| 2.054 | H | H | H | OCF₃ | H | CH₂CH₃ |
| 2.055 | H | H | Br | H | H | H |
| 2.056 | H | H | Br | H | H | CH₃ |
| 2.057 | H | H | Br | H | H | CH₂CH₃ |
| 2.058 | H | H | F | H | H | H |
| 2.059 | H | H | F | H | H | CH₃ |
| 2.060 | H | Cl | H | 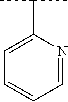 | H | H |
| 2.061 | H | Cl | H | 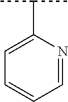 | H | CH₃ |
| 2.062 | H | Cl | H | 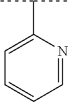 | H | CH₂CH₃ |
| 2.063 | H | Cl | H | 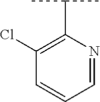 | H | H |
| 2.064 | H | Cl | H | 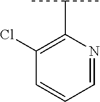 | H | CH₃ |
| 2.065 | H | Cl | H | 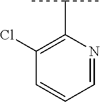 | H | CH₂CH₃ |
| 2.066 | H | Cl | H | 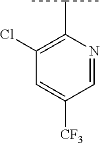 | H | H |
| 2.067 | H | Cl | H | 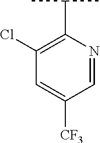 | H | CH₃ |
| 2.068 | H | Cl | H | 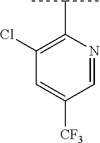 | H | CH₂CH₃ |
| 2.069 | H | Cl | H | 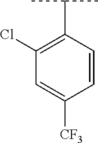 | H | H |
| 2.070 | H | Cl | H | 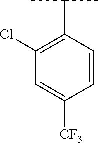 | H | CH₃ |
| 2.071 | H | Cl | H | 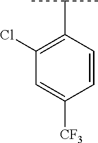 | H | CH₂CH₃ |
| 2.072 | H | Cl | H | 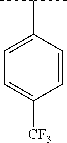 | H | H |
| 2.073 | H | Cl | H | 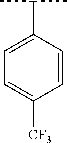 | H | CH₃ |
| 2.074 | H | Cl | H | 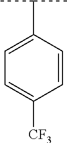 | H | CH₂CH₃ |
| 2.075 | H | Cl | H | 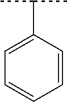 | H | H |
| 2.076 | H | Cl | H | 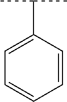 | H | CH₃ |
| 2.077 | H | Cl | H | 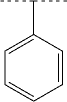 | H | CH₂CH₃ |
| 2.078 | H | Cl | H | 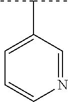 | H | H |
| 2.079 | H | Cl | H | 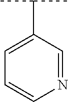 | H | CH₃ |
| 2.080 | H | Cl | H | 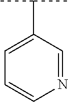 | H | CH₂CH₃ |
| 2.081 | H | Cl | H | 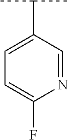 | H | H |
| 2.082 | H | Cl | H | 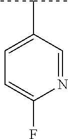 | H | CH₃ |
| 2.083 | H | Cl | H | 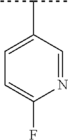 | H | CH₂CH₃ |
| 2.084 | H | Br | H | 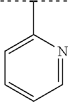 | H | H |
| 2.085 | H | Br | H | 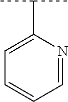 | H | CH₃ |
| 2.086 | H | Br | H | 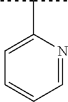 | H | CH₂CH₃ |
| 2.087 | H | Br | H | 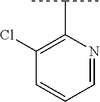 | H | H |
| 2.088 | H | Br | H | 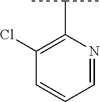 | H | CH₃ |
| 2.089 | H | Br | H | 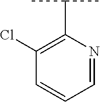 | H | CH₂CH₃ |
| 2.090 | H | Br | H | 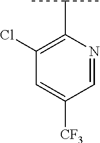 | H | H |
| 2.091 | H | Br | H | 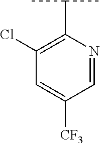 | H | CH₃ |
| 2.092 | H | Br | H | 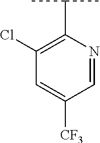 | H | CH₂CH₃ |
| 2.093 | H | Br | H | 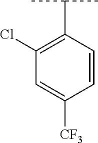 | H | H |
| 2.094 | H | Br | H | 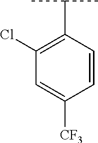 | H | CH₃ |
| 2.095 | H | Br | H | 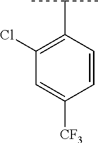 | H | CH₂CH₃ |
| 2.096 | H | Br | H | 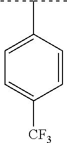 | H | H |
| 2.097 | H | Br | H | 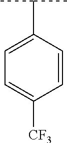 | H | CH₃ |
| 2.098 | H | Br | H | 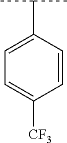 | H | CH₂CH₃ |
| 2.099 | H | Br | H | 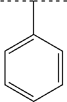 | H | H |
| 2.100 | H | Br | H | 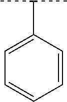 | H | CH₃ |
| 2.101 | H | Br | H | 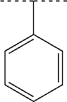 | H | CH₂CH₃ |
| 2.102 | H | Br | H | 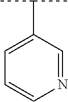 | H | H |
| 2.103 | H | Br | H | 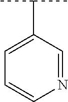 | H | CH₃ |
| 2.104 | H | Br | H | 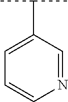 | H | CH₂CH₃ |
| 2.105 | H | Br | H | 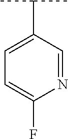 | H | H |
| 2.106 | H | Br | H | 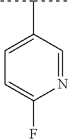 | H | CH₃ |
| 2.107 | H | Br | H | 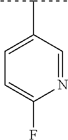 | H | CH₂CH₃ |
| 2.108 | H | CF₃ | H | 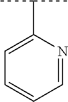 | H | H |
| 2.109 | H | CF₃ | H | 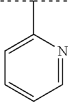 | H | CH₃ |
| 2.110 | H | CF₃ | H | 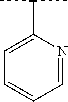 | H | CH₂CH₃ |
| 2.111 | H | CF₃ | H | 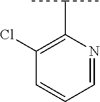 | H | H |
| 2.112 | H | CF₃ | H | 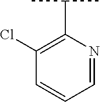 | H | CH₃ |
| 2.113 | H | CF₃ | H | 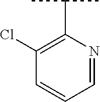 | H | CH₂CH₃ |
| 2.114 | H | CF₃ | H | 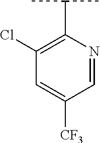 | H | H |
| 2.115 | H | CF₃ | H | 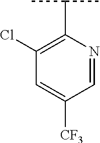 | H | CH₃ |
| 2.116 | H | CF₃ | H | 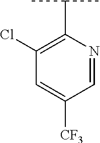 | H | CH₂CH₃ |
| 2.117 | H | CF₃ | H | 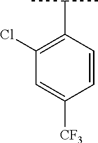 | H | H |
| 2.118 | H | CF₃ | H | 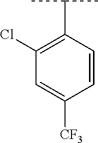 | H | CH₃ |
| 2.119 | H | CF₃ | H | 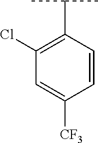 | H | CH₂CH₃ |
| 2.120 | H | CF₃ | H | 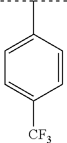 | H | H |
| 2.121 | H | CF₃ | H | 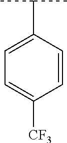 | H | CH₃ |
| 2.122 | H | CF₃ | H | 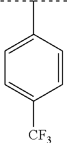 | H | CH₂CH₃ |
| 2.123 | H | CF₃ | H | 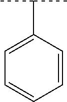 | H | H |
| 2.124 | H | CF₃ | H | 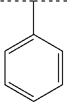 | H | CH₃ |
| 2.125 | H | CF₃ | H | 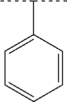 | H | CH₂CH₃ |
| 2.126 | H | CF₃ | H | 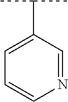 | H | H |
| 2.127 | H | CF₃ | H | 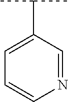 | H | CH₃ |
| 2.128 | H | CF₃ | H | 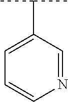 | H | CH₂CH₃ |
| 2.129 | H | CF₃ | H | 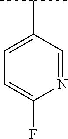 | H | H |
| 2.130 | H | CF₃ | H | 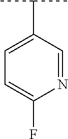 | H | CH₃ |
| 2.131 | H | CF₃ | H | 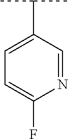 | H | CH₂CH₃ |

The compounds of formula I according to the invention are preventively and/or curatively valuable active ingredients in the field of pest control, even at low rates of application, which have a very favorable biocidal spectrum and are well tolerated by warm-blooded species, fish and plants. The compounds of formula I are safe towards non-target species, such as bees, and accordingly have a good toxicity profile. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the abovementioned animal pests are:

from the order Acarina, for example,

Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.,

from the order Anoplura, for example,

Haematopinus spp., Linognathus spp., Pediculus spp., Pemphigus spp. and Phylloxera spp., from the order Coleoptera, for example,

Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.,

from the order Diptera, for example,

Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.,

from the order Hemiptera, for example,

Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Euschistus spp. (stinkbugs), Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens, Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris,

from the order Hymenoptera, for example,

Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.,

from the order Isoptera, for example,

Coptotermes spp, Corniternes cumulans, Incisitermes spp, Macrotermes spp, Mastotermes spp, Microtermes spp, Reticulitermes spp., Solenopsis geminate

from the order Lepidoptera, for example,

Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Gra-pholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.,

from the order Mallophaga, for example,

Damalinea spp. and Trichodectes spp.,

from the order Orthoptera, for example,

Blatta spp., Blattella spp., Gryllotalpa spp., Leucophaea maderae, Locusta spp., Neocurtilla hexadactyla, Periplaneta spp., Scapteriscus spp, and Schistocerca spp.,

from the order Psocoptera, for example,

Liposcelis spp.,

from the order Siphonaptera, for example,

Ceratophyllus spp., Ctenocephalides spp. and Xenopsylla cheopis,

from the order Thysanoptera, for example,

Calliothrips phaseoli, Frankliniella spp., Heliothrips spp, Hercinothrips spp., Parthenothrips spp, Scirtothrips aurantii, Sericothrips variabilis, Taeniothrips spp., Thrips spp,

from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum, beet, such as sugar or fodder beet, fruit, for example pomaceous fruit, stone fruit or soft fruit, such as apples, pears, plums, peaches, almonds, cherries or berries, for example strawberries, raspberries or blackberries, leguminous crops, such as beans, lentils, peas or soya, oil crops, such as oilseed rape, mustard, poppies, olives, sunflowers, coconut, castor, cocoa or ground nuts, cucurbits, such as pumpkins, cucumbers or melons, fibre plants, such as cotton, flax, hemp or jute, citrus fruit, such as oranges, lemons, grapefruit or tangerines, vegetables, such as spinach, lettuce, asparagus, cabbages, carrots, onions, tomatoes, potatoes or bell peppers, Lauraceae, such as avocado, Cinnamonium or camphor, and also tobacco, nuts, coffee, eggplants, sugarcane, tea, pepper, grapevines, hops, the plantain family, latex plants and ornamentals.

In an embodiment, the active ingredients according to the invention are especially suitable for controlling stink bugs. Stink bugs are from the order Hemiptera and examples are: Acrosternum spp., Acrosternum hilare, Antestiopsis spp., Antestiopsis orbitalus, Dichelops spp., Dichelops furcatus, Dichelops melacanthus, Dichelops melacanthus, Edessa spp., Edessa meditabunda, Euchistus spp., Eurygaster spp., Euschistus spp., Euschistus heros, Euschistus servus, Halyomorpha spp., Halyomorpha halys, Murgantia spp., Nezara spp., Nezara antennata, Nezara hilare, Nezara viridula, Oebalus spp., Oebalus mexicana, Oebalus poecilus, Oebalus pugnase, Oebalus pugnax, Piezodorus spp., Piezodorus guildinii, Plautia crossota, Scotinophara spp., Scotinophara coarctata, Scotinophara lurida, Scotinophora spp., Thyanta spp., Tibraca spp. In a preferred embodiment, stinkbugs are, e.g. Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus servus), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase), and Scotinophara spp. (e.g. Scotinophara lurida, Scotinophara coarctatd). Preferred targets include Antestiopsis orbitalus, Dichelops furcatus, Dichelops melacanthus, Euchistus heros, Euschistus servus, Nezara viridula, Nezara hilare, Piezodorus guildinii, Halyomorpha halys. In one embodiment the stinkbug target is Nezara viridula, Piezodorus spp., Acrosternum spp, Euchistus heros. The compounds of the invention are particularly effective against Euschistus and in particular Euchistus heros.

In a further embodiment, the active ingredients according to the invention are especially suitable for controlling a pest selected from Adoxophyes spp., Agrotis spp., Anticarsia spp., Apamea spp., Chilo spp., Cnaphalocrocis spp., Diaphania spp., Earias spp., Elasmopalpus spp., Epinotia spp., Eupoecilia spp., Euxoa spp., Feltia spp., Grapholita spp., Helicoverpa spp., Heliothis spp., Homoeosoma spp., Keiferia spp., Laphygma spp., Leucinodes spp., Lobesia spp., Lymantria spp., Mamestra spp., Marasmia spp., Maruca spp., Neoleucinodes spp., Oria spp., Ostrinia spp., Pectinophora spp., Perileucoptera spp., Phthorimaea spp., Phyllocnistis spp., Pieris spp., Plusia spp., Plutella spp., Prodenia spp., Pseudaletia spp., Pseudoplusia spp., Rachiplusia spp., Scirpophaga spp., Sesamia spp., Spodoptera spp., and Trichoplusia spp.; preferably Agrotis spp., Earias spp., Elasmopalpus spp., Helicoverpa spp., Heliothis spp., Pectinophora spp., and Spodoptera spp.,

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species, cyst-forming nematodes, Globodera rostochiensis and other Globodera species, Heterodera avenae, Heterodera glycines, Heterodera schachtii, Heterodera trifolii, and other Heterodera species, Seed gall nematodes, Anguina species, Stem and foliar nematodes, Aphelenchoides species, Sting nematodes, Belonolaimus longicaudatus and other Belonolaimus species, Pine nematodes, Bursaphelenchus xylophilus and other Bursaphelenchus species, Ring nematodes, Criconema species, Criconemella species, Criconemoides species, Mesocriconema species, Stem and bulb nematodes, Ditylenchus destructor, Ditylenchus dipsaci and other Ditylenchus species, Awl nematodes, Dolichodorus species, Spiral nematodes, Heliocotylenchus multicinctus and other Helicotylenchus species, Sheath and sheathoid nematodes, Hemicycliophora species and Hemicriconemoides species, Hirshmanniella species, Lance nematodes, Hoploaimus species, false rootknot nematodes, Nacobbus species, Needle nematodes, Longidorus elongatus and other Longidorus species, Pin nematodes, Pratylenchus species, Lesion nematodes, Pratylenchus neglectus, Pratylenchus penetrans, Pratylenchus curvitatus, Pratylenchus goodeyi and other Pratylenchus species, Burrowing nematodes, Radopholus similis and other Radopholus species, Reniform nematodes, Rotylenchus robustus, Rotylenchus reniformis and other Rotylenchus species, Scutellonema species, Stubby root nematodes, Trichodorus primitivus and other Trichodorus species, Paratrichodorus species, Stunt nematodes, Tylenchorhynchus claytoni, Tylenchorhynchus dubius and other Tylenchorhynchus species, Citrus nematodes, Tylenchulus species, Dagger nematodes, Xiphinema species, and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae, Arion (A. ater, A. circumscriptus, A. hortensis, A. rufus), Bradybaenidae (Bradybaena fruticum), Cepaea (C. hortensis, C. Nemoralis), ochlodina, Deroceras (D. agrestis, D. empiricorum, D. laeve, D. reticulatum), Discus (D. rotundatus), Euomphalia, Galba (G. trunculata), Helicelia (H. itala, H. obvia), Helicidae Helicigona arbustorum), Helicodiscus, Helix (H. aperta), Limax (L. cinereoniger, L. flavus, L. marginatus, L. maximus, L. tenellus), Lymnaea, Milax (M. gagates, M. marginatus, M. sowerbyi), Opeas, Pomacea (P. canaticulata), Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae, or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A, or insecticidal proteins of bacteria colonising nematodes, for example Photorhabdus spp. or Xenorhabdus spp., such as Photorhabdus luminescens, Xenorhabdus nematophilus, toxins produced by animals, such as scorpion toxins, arachnid toxins, wasp toxins and other insect-specific neurotoxins, toxins produced by fungi, such as Streptomycetes toxins, plant lectins, such as pea lectins, barley lectins or snowdrop lectins, agglutinins, proteinase inhibitors, such as trypsin inhibitors, serine protease inhibitors, patatin, cystatin, papain inhibitors, ribosome-inactivating proteins (RIP), such as ricin, maize-RIP, abrin, luffin, saporin or bryodin, steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by b-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin), YieldGard Rootworm® (maize variety that expresses a Cry3Bb1 toxin), YieldGard Plus® (maize variety that expresses a Cry1Ab and a Cry3Bb1 toxin), Starlink® (maize variety that expresses a Cry9C toxin), Herculex I® (maize variety that expresses a Cry1Fa2 toxin and the enzyme phosphinothricine N-acetyltransferase (PAT) to achieve tolerance to the herbicide glufosinate ammonium), NuCOTN 33B® (cotton variety that expresses a CrylAc toxin), Bollgard I® (cotton variety that expresses a CrylAc toxin), Bollgard II® (cotton variety that expresses a CrylAc and a Cry2Ab toxin), VipCot® (cotton variety that expresses a Vip3A and a Cry1Ab toxin), NewLeaf® (potato variety that expresses a Cry3A toxin), NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603 x MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins, stilbene synthases, bibenzyl synthases, chitinases, glucanases, the so-called “pathogenesis-related proteins” (PRPs, see e.g. EP-A-0 392 225), antipathogenic substances produced by microorganisms, for example peptide antibiotics or heterocyclic antibiotics (see e.g. WO 95/33818) or protein or polypeptide factors involved in plant pathogen defense (so-called “plant disease resistance genes”, as described in WO 03/000906).

Further areas of use of the compositions according to the invention are the protection of stored goods and store ambients and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors, see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents. A further object of the invention is therefore a substrate selected from nonwoven and fabric material comprising a composition which contains a compound of formula I.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 03/034823, U.S. Pat. No. 5,631,072, WO 2005/64072, WO 2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleotera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, |
| | | Netleaf hackberry |
| | Oberea ocellata | Sumac, Apple, Peach, Plum, |
| | | Pear, Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, |
| | | Beech, Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black |
| | | cherry, Elm, Mulberry, Mountain-ash |
| | Pseudopityophthorus pruinosus | Oak, American beech, Black |
| | | cherry, Chickasaw plum, Chestnut, |
| | | Maple, Hickory, Hornbeam, |
| | | Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, |
| | | Black Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass Ataenius, A. spretulus), Maladera spp. (e.g. Asiatic garden beetle, M. castanea) and Tomarus spp.), ground pearls (Margarodes spp.), mole crickets (tawny, southern, and short-winged, Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.,
Of the order Mallophagida: Trimenopon spp., Menopon spp., Trinoton spp., Bovicola spp., Werneckiella spp., Lepikentron spp., Damalina spp., Trichodectes spp. and Felicola spp.,
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.,
Of the order Siphonapterida, for example Pulex spp., Ctenocephalides spp., Xenopsylla spp., Ceratophyllus spp.,
Of the order Heteropterida, for example Cimex spp., Triatoma spp., Rhodnius spp., Panstrongylus spp.,
Of the order Blattarida, for example Blatta orientalis, Periplaneta americana, Blattela germanica and Supella spp.,
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.,
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants or additives, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate, salts of alkylarylsulfonates, such as calcium dodecylbenzenesulfonate, alkylphenol/alkylene oxide addition products, such as nonylphenol ethoxylate, alcohol/alkylene oxide addition products, such as tridecylalcohol ethoxylate, soaps, such as sodium stearate, salts of alkylnaphthalenesulfonates, such as sodium dibutylnaphthalenesulfonate, dialkyl esters of sulfosuccinate salts, such as sodium di(2-ethylhexyl)sulfosuccinate, sorbitol esters, such as sorbitol oleate, quaternary amines, such as lauryltrimethylammonium chloride, polyethylene glycol esters of fatty acids, such as polyethylene glycol stearate, block copolymers of ethylene oxide and propylene oxide, and salts of mono- and di-alkylphosphate esters, and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern IIIinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

Emulsifiable Concentrates:
active ingredient: 1 to 95%, preferably 60 to 90%
surface-active agent: 1 to 30%, preferably 5 to 20%
liquid carrier: 1 to 80%, preferably 1 to 35%

Dusts:
active ingredient: 0.1 to 10%, preferably 0.1 to 5%
solid carrier: 99.9 to 90%, preferably 99.9 to 99%

Suspension Concentrates:
active ingredient: 5 to 75%, preferably 10 to 50%
water: 94 to 24%, preferably 88 to 30%
surface-active agent: 1 to 40%, preferably 2 to 30%

Wettable Powders:
active ingredient: 0.5 to 90%, preferably 1 to 80%
surface-active agent: 0.5 to 20%, preferably 1 to 15%
solid carrier: 5 to 95%, preferably 15 to 90%

Granules:
active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of | 3% |
| ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of | 6% |
| ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20%) | 0.5% |
| solution in water | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

In a further aspect, the present invention makes available a pesticidal composition comprising a compound of the first aspect, one or more formulation additives and a carrier.

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula I with active ingredients are preferred (the abbreviation “TX” means “one compound selected from the group consisting of the compounds described in Tables 1 and A (including Table A2) of the present invention”):

an adjuvant selected from the group of substances consisting of petroleum oils (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacry-pyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl O-(methoxyaminothiophosphoryl)salicylate (IUPAC name) (473)+TX, ivermectin [CCN]+TX, jasmolin 1(696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thiocarboxime (1431)+TX, thiofanox (800)+TX, thiometon (801)+TX, thioquinox (1436)+TX, thuringiensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX,

a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX,

a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria brongniartii (54)+TX, Chrysoperla carnea (151)+TX, Cryptolaemus montrouzieri (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopii (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Orius spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema riobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillium lecanii (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of I-dec-5-en-1-yl acetate with I-dec-5-en-1-ol (IUPAC name) (222)+TX, 1-tridec-4-en-1-yl acetate (IUPAC name) (829)+TX, I-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordidin (736)+TX, sulcatol [CCN]+TX, tetradec-11-en-1-yl acetate (IUPAC name) (785)+TX, trimedlure (839)+TX, trimedlure A (839)+TX, trimedlure B (839)+TX, trimedlure B2 (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,

an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin I1 (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone III [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, potassium arsenite [CCN]+TX, potassium thiocyanate [CCN]+TX, prallethrin (655)+TX, precocene I [CCN]+TX, precocene II [CCN]+TX, precocene III [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spiropidion (CCN)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, Cyantraniliprole [736994-63-1]+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX, DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (720)+TX, a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, meta-laxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3][112-65-2](free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzo-lar-S-methyl[135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX, lancotrione [1486617-21-3]+TX, florpyrauxifen [943832-81-3]]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]]+TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX, kappa-tefluthrin [391634-71-2]+TX, fenpicoxamid [517875-34-2]+TX, fluindapyr [1383809-87-7]+TX, alpha-bromadiolone [28772-56-7]+TX, flupyrimin [1689566-03-7]+TX, benzpyrimoxan [1449021-97-9]+TX, acynonapyr [1332838-17-1]+TX, inpyrfluxam [1352994-67-2]+TX, isoflucypram [1255734-28-1]+TX, rescalure [64309-03-1]+TX, fluxametamide [928783-29-3]+TX, tetraniliprole [1229654-66-3]+TX, guadipyr (described in WO2010/060231)+TX, cycloxaprid (described in WO 2005/077934)+TX, Afidopyropen+TX, kappa-bifenthrin+TX, kappa-tefluthrin+TX, Tetrachloraniliprole+TX, aminopyrifen [1531626-08-0]+TX, tyclopyrazoflor [1477919-27-9]+TX, Dichloromezotiaz+TX, Momfluorothrin+TX, Fluopyram+TX, Terpenoid blend+TX, Fluhexafon+TX, Cyclaniliprole+TX, Isocycloseram+TX; dimpropyridaz+TX; and spiropidion [1229023-00-0]+TX; and

microbials including: Acinetobacter twoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®, BioNem-WP®, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide)+TX, Candida butyri+TX, Candida famata+TX, Candida fructus+TX, Candida glabrata+TX, Candida guilliermondii+TX, Candida melibiosica+TX, Candida oleophila strain 0+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (Amykor®+TX, Root Maximizer®)+TX, Myrothecium verrucaria strain AARC-0255 (DiTera)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG)+TX, Paenibacillus polymyxa+TX, Pantoea agglomerans (BlightBan C₉-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus, and Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®, Neemix®)+TX, canola oil (Lilly Miller Vegol®)+TX, Chenopodium ambrosioides near ambrosioides (Requiem®)+TX, Chrysanthemum extract (Crisant)+TX, extract of neem oil (Trilogy®)+TX, essentials oils of Labiatae (Botania®)+TX, extracts of clove rosemary peppermint and thyme oil (Garden Insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®)+TX, and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, Tetradecatrienyl acetate+TX, 13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate, and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®)+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator, and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium, Thirteenth Edition, Editor: C. D. S. TomLin, The British Crop Protection Council], they are described therein under the entry number given in round brackets hereinabove for the particular compound, for example, the compound “abamectin” is described under entry number (1). Where “[CCN]” is added hereinabove to the particular compound, the compound in question is included in the “Compendium of Pesticide Common Names”, which is accessible on the internet [A. Wood, Compendium of Pesticide Common Names, Copyright © 1995-2004], for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound, in that case, the IUPAC name, the IUPAC/Chemical Abstracts name, a “chemical name”, a “traditional name”, a “compound name” or a “development code” is used. “CAS Reg. No” means the Chemical Abstracts Registry Number.

The ratio (by weight) of active ingredient mixture of the compounds of formula I selected from Tables 1 and A with active ingredients described above is from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1 and A and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1 and A and the active ingredients as described above is not essential for working the present invention.

In a further aspect, the present invention provides a combination of active ingredients comprising a compound defined in the first aspect, and one or more further active ingredients (whether chemical or biological).

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula 1. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I. Further, it is hereby made available, a composition comprising a plant propagation material treated with a compound of formula I.

Seed treatment comprises all suitable seed treatment techniques known in the art, such as seed dressing, seed coating, seed dusting, seed soaking and seed pelleting. The seed treatment application of the compound formula I can be carried out by any known methods, such as spraying or by dusting the seeds before sowing or during the sowing/planting of the seeds.

The compounds of the invention can be distinguished from other similar compounds by virtue of greater efficacy at low application rates, which can be verified by the person skilled in the art using the experimental procedures outlined in the Examples below, using lower concentrations if necessary, for example 10 ppm, 5 ppm, 2 ppm, 1 ppm or 0.2 ppm; or lower application rates, such as 300, 200 or 100, mg of A1 per m².

An aspect of the present invention is a method of controlling insects, acarines, nematodes or molluscs which comprises applying an insecticidally, acaricidally, nematicidally or molluscicidally effective amount of a compound of formula I defined the first aspect, or a composition containing a compound of formula I defined the first aspect, to a pest, a locus of pest, preferably a plant, to a plant susceptible to attack by a pest or to plant propagation material thereof, such as a seed, provided if the the control were on a human or animal body, then it is non-therapeutical.

A further aspect is a plant propagation material comprising by way of treatment or coating one or more compounds of formula I defined the first aspect, optionally also comprising a colour pigment.

The compounds of the present invention as well as providing pesticidal activity, may possess improved insecticidal properties, such as improved efficacy for example, at lower rates or faster, improved selectivity, reduced toxicity, lower tendency to generate resistance or activity against a broader range of pests. Compounds may be more advantageously formulated or better physchem to provide more efficient delivery and retention at sites of action, or may have less persistence in the environment.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

The following Examples serve to illustrate the invention. They do not limit the invention. Temperatures are given in degrees Celsius; mixing ratios of solvents are given in parts by volume.

### PREPARATORY EXAMPLES

“Mp” means melting point in ° C. Free radicals represent methyl groups. ¹H and ¹⁹F NMR measurements were recorded on Brucker 400 MHz or 300 MHz spectrometers, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ and/or (M−H)⁻.

### LCMS Methods:

### Method A—Standard: (SQD-ZDQ-ZCQ)

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 □m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85.

### a) Synthesis of Intermediates

### Example I1: Preparation of N-[(2-chlorothiazol-5-yl)methyl]-1-methyl-pyrazol-3-amine

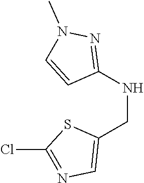

### Methode A:

To a stirred solution of 1-methylpyrazol-3-amine (CAS 1904-31-0), 3.36 g, 34.6 mmol, 2 equiv.) in dimethylformamide (20 mL) at RT was added sodium hydride (60 mass % in oil) (1.38 g, 34.5 mmol, 2 equiv.). After addition, the reaction mixture was stirred for 30 minutes at the same temperature. Then 2-chloro-5-(chloromethyl)thiazole (CAS 105827-91-6), 3.00 g, 17.3 mmol, 1 equiv.) dissolved in dimethylformamide (10 mL) was added to the reaction mixture at room temperature and then, the reaction mixture was stirred at 70° C. for 3 h. The reaction was monitored by TLC and after completion, the reaction mixture was diluted with ice water and ethyl acetate. All volatiles were removed under vacuum and the solid was dissolved in ethyl acetate/water and filtered over Hyflo. The organic layer was separated and the water layer was extracted, two times, with ethyl acetate. The combined organic layers were washed with brine, dried over magnesium sulfate and concentrated under vacuum. The residue was purified by flash chromatography using a gradient of ethyl acetate in cyclohexane (0 to 100%) to give the title compound (14% yield).

¹H NMR (400 MHz, CDCl₃) □ ppm 7.48 (1H, s), 7.20 (1H, s), 5.58 (1H, s), 4.52 (2H, s), 3.82 (3H, s).

### Methode B:

### Step I1-A: tert-butyl N-(1-methylpyrazol-3-yl)carbamate

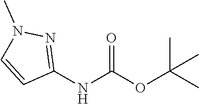

a 500 ml 3-necked flask was charged with di-t-butyl dicarbonate (36.32 g, 38 mL, 164.7 mmol) and hexanes (66.6 mL). The colourless solution was heated to reflux at 59° C., then a solution of 1-methylpyrazol-3-amine (CAS 1904-31-0), 10 g, 103.0 mmol) in ethyl acetate (18.0 g, 20 mL, 204 mmol) was added dropwise over an hour period. The Heating was kept one hour more after end of addition, and the grey solution was allowed to cool down slowly over night.

The white precipitate formed was filtered and rinsed with Hexane to give the tittle compound (14.3 g, 70.4% Yield). ¹H NMR (400 MHz, Solvent) δ ppm 1.53 (s, 9H), 3.81 (s, 3H), 6.25-6.61 (m, 1H), 7.23 (d, 1H), 7.66 (br s, 1H).

### Step I1-B: Synthesis of tert-butyl N-[(2-chlorothiazol-5-yl)methyl]-N-(1-methylpyrazol-3-yl)carbamate

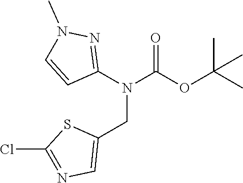 [SMILES: Cn1ccc(N(Cc2cnc(Cl)s2)C(=O)OC(C)(C)C)n1]

To a stirred solution of tert-butyl N-(1-methylpyrazol-3-yl)carbamate (14.2 g, 72.0 mmol) in dimethylformamide (67.0 g, 71.0 mL, 915 mmol) at 0° C. was added sodium hydride (60 mass % in oil) (3.24 g, 81.0 mmol) portion wise and after addition reaction mixture was stirred for 30 minute at the same temperature. Then 2-chloro-5-(chloromethyl)thiazole (CAS 105827-91-6, 16.5 g, 95.4 mmol, 1.32) was added to the reaction mixture at 0° C. and stirred at room temperature for 3 h. After completion of the reaction mixture was diluted with ice water and extracted with ethyl acetate (3×). the combinated organic layers were washed with brine, dried over sodium sulphate and concentrated. The crude was purified by silica gel column chromatography (330 g) using a gradient of ethyl acetate in cyclohexane (0 to 50%) to give the title compound (20.6 g, 87.0% Yield). LC-MS (method A): 329 (M+1)⁺, retention time 1.04 min.

### Step I1-C: Synthesis of N-[(2-chlorothiazol-5-yl)methyl]-1-methyl-pyrazol-3-amine

To a solution of tert-butyl N-[(2-chlorothiazol-5-yl)methyl]-N-(1-methylpyrazol-3-yl)carbamate (Step I1-B, 20.6 g, 62.7 mmol) in dichloromethane (66.25 g, 50 mL, 778 mmol) was added trifluoroacetic acid (72.2 g, 48.4 mL, 627 mmol). The solution was stirred for 18 hours at room temperature. Then the trifluoroacetic acid was neutralized by addition of a saturated solution of potassium carbonate. The water layer was extracted, three times, with ethyl acetate. The combined organic layers were washed with brine dried on magnesium sulfate and concentrated under vacuum. The residue was used without extra purification for the next step.

¹H NMR (400 MHz, CDCl₃) □ ppm 7.48 (1H, s), 7.20 (1H, s), 5.58 (1H, s), 4.52 (2H, s), 3.82 (3H, s).

### Example I1: Preparation of bis(2,4,6-trichlorophenyl) 2-(3,5-dichlorophenyl)propanedioate

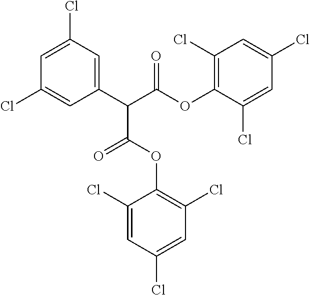 [SMILES: O=C(Oc1c(Cl)cc(Cl)cc1Cl)C(C(=O)Oc1c(Cl)cc(Cl)cc1Cl)c1cc(Cl)cc(Cl)c1]

### Step I1-A: Synthesis of dimethyl 2-(3,5-dichlorophenyl)propanedioate

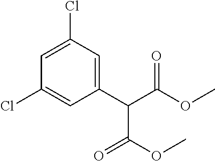 [SMILES: COC(=O)C(C(=O)OC)c1cc(Cl)cc(Cl)c1]

To a solution of 1,3-dichloro-5-iodo-benzene in 1,4-dioxane (160 mL) was added Copper(I) iodide (0.661 g), pyridine-2-carboxylic acid (0.812 g), and cesium carbonate (32 g) under Argon. Then dimethyl propanedioate (15 g) was added at room temperature via dropping funnel. The mixture was heated to 90° C. for 5h. After completion of the reaction mixture was diluted with water and extracted with ethyl acetate (2×). the combinated organic layers were washed with brine, dried over sodium sulphate and concentrated. The crude was purified by silica gel column chromatography (3RF 200) using a gradient of ethyl acetate (0 to 10%) in cyclohexane to give the title compound (11.75 g, 77.1% Yield). LC-MS (method A): 277 (M+1)⁺. 275 (M−1)⁺ retention time 1.02 min.

### Step I1-B: Synthesis of 2-(3,5-dichlorophenyl)propanedioic acid

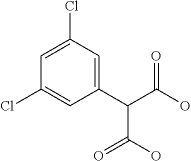 [SMILES: O=C(O)C(C(=O)O)c1cc(Cl)cc(Cl)c1]

Dimethyl 2-(3,5-dichlorophenyl)propanedioate (11.75 g) was solved in Sodium hydroxide 1N (340 ml, 8 equiv.) and the reaction was stirred over night at 50° C. After completion of the reaction mixture was diluted with dichloromethane and the organic layers were separated. The aqueous layer was additionned of a solution of hydrogen chloride (2N) at −5/0° C. until pH 2. In addition, at pH 2, Sodium chloride and dichloromethane were additioned to the water layer, then the mixture was stirred for about 60 min at <0° C. until a solid appeared. After filtration, the solid was once washes with cold water and once with Methyl-tert-butylether and give the title compound (8.56 g). The compound is instable in solvent and the NMR shows only the decarboxylated compound.

### Step I1-C: Synthesis of bis(2,4,6-trichlorophenyl) 2-(3,5-dichlorophenyl)propanedioate

To a solution of 2-(3,5-dichlorophenyl)propanedioic acid (8.50 g) in dichloromethane (140 mL) was added dimethyl formamide (0.3 mL) at 5° C. Then oxalyl dichloride (7.2 mL) was added dropwise. The reaction was stirred at room temperature for 2h. 2 extra ml oxalyl dichloride was added and stirred for an extra hour at room temperature. At 5° C. 2,4,6-trichlorophenol (14.8 g) was added. The reaction was stirred over night at room temperature. All volatiles were removed under vacuum and the solid was additionned of cold methanol. The solid was filtered off to give the title compound (16.37 g).

¹H NMR (400 MHz, CDCl₃) □ ppm 7.60 (2H, s), 7.48 (1H, s), 7.42 (4H, s), 5.26 (1H, s).

### Example I2: Preparation of diphenyl 2-[3-bromo-5-(trifluoromethyl)phenyl]propanedioate

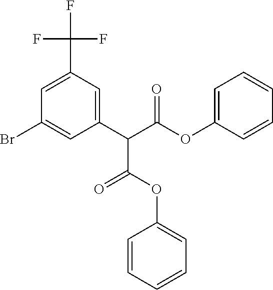 [SMILES: O=C(Oc1ccccc1)C(C(=O)Oc1ccccc1)c1cc(Br)cc(C(F)(F)F)c1]

### Step I2-A: Synthesis of diethyl 2-[3-bromo-5-(trifluoromethyl)phenyl]propanedioate

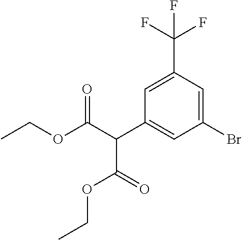 [SMILES: CCOC(=O)C(C(=O)OCC)c1cc(Br)cc(C(F)(F)F)c1]

The compound was synthesised using the similar protocol described in organic letters, 2007, Vol. 9, No. 17, 3469-3472 and using the available 1-Bromo-3-iodo-5-(trifluoromethyl)benzene (CAS: 481075-59-6). LC-MS (method A): 385 (M+1)⁺, 383 (M−1) retention time 1.17 min

### Step I2-B: Synthesis of disodium; 2-[3-bromo-5-(trifluoromethyl)phenyl]propanedioate

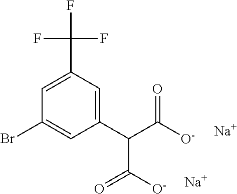 [SMILES: O=C([O-])C(C(=O)[O-])c1cc(Br)cc(C(F)(F)F)c1 ; [Na+] ; [Na+]]

To a solution of diethyl 2-[3-bromo-5-(trifluoromethyl)phenyl]propanedioate (5 g, 13.05 mmol) in ethanol (1.7 mL) was added sodium hydroxide (1N aq., 27.40 mL, 27.40 mmol). The mixture was stirred 3h at 60° C. and concentrated under vacuum. The mixture was co-evaporated with toluene (3×), then, finally evaporated to dryness to give the tittle compound in mixture with the decarboxylated analogue. The mixture was used without extra purification for the next step.

### Step I2-C: Synthesis of phenyl 2-[3-bromo-5-(trifluoromethyl)phenyl]acetate

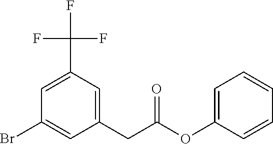 [SMILES: O=C(Cc1cc(Br)cc(C(F)(F)F)c1)Oc1ccccc1]

At room temperature, a vial was charged with disodium; 2-[3-bromo-5-(trifluoromethyl)phenyl]propanedioate (4.54 g, from Step I1-B), 10.4 mmol) dissolved in dichloromethane (45.4 mL), Then were added Phenol (1.39 g) and dimethylamino pyridine (0.180 g). Then the solution was cooled at 0° C. and EDC-HCl (3.46 g, 17.7 mmol) was slowly added portionwise. The mixture was stirred at room temperature for 2.5h, quenched with aqueous diluted with a saturated solution of sodium hydrogenocarbonate, dry with MgSO4, filter and volatils were remove under vacuum. The crude was purified by silica gel column chromatography (330 g SiO₂) using a gradient of ethyl acetate (0 to 20%) in heptane to give the title compound (70% purity) that was used without extra purification for the next step.

### Step I2-D: Synthesis of diphenyl 2-[3-bromo-5-(trifluoromethyl)phenyl]propanedioate

In a dry 50 ml 2-necked round-bottom flask was charged at RT with phenyl 2-[3-bromo-5-(trifluoromethyl)phenyl]acetate (2.04 g, 5.671 mmol) diluted into THE (8.15 mL) under Argon. The resulting colourless solution was cooled down to −78° C. and Potassium bis(trimethylsilyl) amide solution (20% in THF, 19 mL, 17.01 mmol) was added dropwise with a syringe over 5 min to afford a yellow solution. It was stirred at −78° C. for 20 min. Then Phenyl chloroformate (1.1 mL) was added dropwise with a syringe and the resulting yellow solution was stirred at −78° C. for 2h. Then, at −70° C., 10 ml of a saturated solution ammonium chloride were added and temperature was allowed to reach room temperature. The aqueous phase was extracted with ethyl acetate (2×). the combinated organic layers were washed with brine, dried over sodium sulphate and concentrated. The crude was purified by silica gel column chromatography using a gradient of ethyl acetate (0 to 20%) in heptane to give the title compound. ¹H NMR (400 MHz, CDCl₃-d) δ ppm 5.14 (s, 1H), 7.16 (m, 4H), 7.32 (m, 2H), 7.44 (m, 4H), 7.85 (d, 2H), 8.0 (s, 1H)

### Example 13: Preparation of bis(phenyl) 2-[3-[3-chloro-5-(trifluoromethyl)-2-pyridyl]phenyl]propanedioate

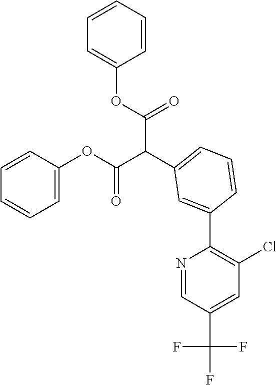 [SMILES: O=C(Oc1ccccc1)C(C(=O)Oc1ccccc1)c1cccc(-c2ncc(C(F)(F)F)cc2Cl)c1]

### Step I3-A: Synthesis of ethyl 2-[3-(4,4,5,5-tetramethyl-1,3,2-dioxaborolan-2-yl)phenyl]acetate

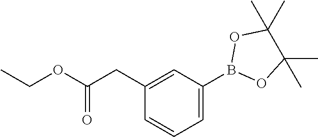 [SMILES: CCOC(=O)Cc1cccc(B2OC(C)(C)C(C)(C)O2)c1]

In a dry 500 ml flask was charged at room temperature with ethyl 2-(3-bromophenyl)acetate (CAS 14062-30-7, 10 g) dissolved in Dioxane (123 mL) was added of 4,4,5,5-tetramethyl-2-(4,4,5,5-tetramethyl-1,3,2-dioxaborolan-2-yl)-1,3,2-dioxaborolane (20.9 g), potassium acetic acid (8.7 g), Chloro(η3-syn-crotyl)(tricyclohexylphosphine)palladium (0.79 g). The reaction mixture was heated up to 90° C. and stirred over night. Then, water was added and the aqueous phase was extracted with ethyl acetate (2×). the combinated organic layers were washed with water and brine, dried over sodium sulphate and concentrated. The crude was purified by silica gel column chromatography RF200 (120 g column; cyclo Hexane/EA) to give the title compound. LC-MS (method A): 292 (M+1)⁺ retention time 1.14 min.

### Step I3-B: Synthesis of ethyl 2-[3-[3-chloro-5-(trifluoromethyl)-2-pyridyl]phenyl]acetate

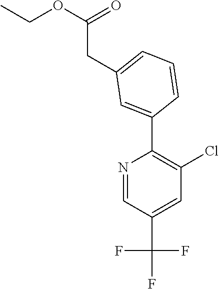 [SMILES: CCOC(=O)Cc1cccc(-c2ncc(C(F)(F)F)cc2Cl)c1]

In a dry 25 ml 2-necked round-bottom flask was charged at room temperature with ethyl 2-[3-(4,4,5,5-tetramethyl-1,3,2-dioxaborolan-2-yl)phenyl]acetate (12.75 g) dissolved in THE (87.9 mL) was added of Bis(triphenylphosphinepalladium(II)dichloride (0.77 g), potassium carbonate (18.48 g) dissolved in water (43.45 mL) and 2,3-dichloro-5-(trifluoromethyl)pyridine (9.49 g). The reaction mixture was heated 70° C. for 4h. Then, water was added and the aqueous phase was extracted with ethyl acetate (2×). the combinated organic layers were washed with water and brine, dried over sodium sulphate and concentrated. The crude was purified by silica gel column chromatography RF200 (40 g column; cyclohexane/EA) to give the title compound. LC-MS (method A): 345 (M+1)⁺ retention time 1.18 min.

### Step I3-C: Synthesis of 2-[3-[3-chloro-5-(trifluoromethyl)-2-pyridyl]phenyl]acetic acid

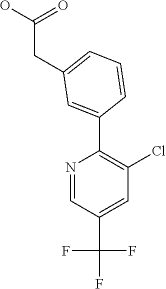 [SMILES: O=C(O)Cc1cccc(-c2ncc(C(F)(F)F)cc2Cl)c1]

ethyl 2-[3-[3-chloro-5-(trifluoromethyl)-2-pyridyl]phenyl]acetate (1.16 g) was solved in a mixture of tetrahydrofuran (10 mL) and water (5 mL) then Lithium hydroxide was added. The reaction was stirred 1h at room temperature. After completion of the reaction mixture was diluted with water and acidified with HCl 1M (pH 2). the aqueous phase was extracted with ethyl acetate (2×). the combinated organic layers were washed with water and brine, dried over sodium sulphate and concentrated to give the title compound. The compound was used without extra purification for the next step. LC-MS (method A): 316 (M+1)⁺ retention time 0.96 min.

### Step I3-D: Synthesis of phenyl 2-[3-[3-chloro-5-(trifluoromethyl)-2-pyridyl]phenyl]acetate

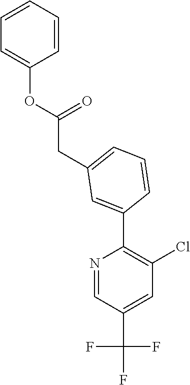 [SMILES: O=C(Cc1cccc(-c2ncc(C(F)(F)F)cc2Cl)c1)Oc1ccccc1]

In a dry 250 ml 2-necked round-bottom flask was charged at room temperature with 2-[3-[3-chloro-5-(trifluoromethyl)-2-pyridyl]phenyl]acetic acid (7.89 g) dissolved in dichloromethane (99.9 mL) was added of 3-(ethyliminomethyleneamino)-N,N-dimethyl-propan-1-amine; hydrochloride (1.44 g), phenol (8.78 mL) and a catalytic amount of dimethylamonopyridine (0.3 g). The reaction mixture was let running over weekend. Then, water was added and the aqueous phase was extracted with ethyl acetate (2×). the combinated organic layers were washed with water and brine, dried over sodium sulphate and concentrated. The crude was purified by silica gel column chromatography RF200 (80 g column; cyclohexane/EA) to give the title compound. The compound was used without extra purification for the next step. LC-MS (method A): 392 (M+1)⁺ retention time 1.21 min.

### Step I3-E: Synthesis of bis(phenyl) 2-[3-[3-chloro-5-(trifluoromethyl)-2-pyridyl]phenyl]propanedioate

This compound was synthesised using similar conditions as described in Step 2-D. LC-MS (method A): 512 (M+1)⁺, 383 (M−1) retention time 1.36 min.

### Example I4: Preparation of 3,3-dichloro-2-(3,5-dichlorophenyl)prop-2-enoic acid

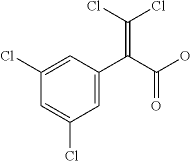 [SMILES: O=C(O)C(=C(Cl)Cl)c1cc(Cl)cc(Cl)c1]

### Step I4-A: Synthesis of ethyl 2-(3,5-dichlorophenyl)-2-oxo-acetate

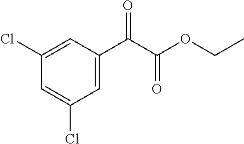

To a 3-necked 50 mL round bottomed flask (RBF) under argon were added 1-bromo-3,5-dichlorobenzene (1.052 equiv., 4.107 g), THE (0.625 mL/mmol, 9.41 g, 10.5848 mL). To the resulting pale yellow solution isopropylmagnesium chloride (1.043 equiv., 8.6 g, 8.8 mL) was added dropwise. The solution mixture became yellow to green. The temperature was controlled around 40° C. In a separate flask under argon were added diethyl oxalate (2500 mg, 2.5 g, 2.323 mL) in tetrahydrofuran (0.4 mL/mmol, 6.02 g, 6.77 mL). The reaction mixture was cooled to −55° C. Then the solution of the “Grignard reagent” was added dropwise (˜25 min). The reaction mixture was stirred at −55° C. The reaction was allowed to warm up to rt overnight. Water (12 mL) and 10% aqueous HCl (12 mL) were added to the reaction mixture. Then the aqueous layer was extracted twice with ethyl acetate. The combined organic layer were dried over magnesium sulfate, filtered and concentrated under reduced pressure. The crude product was used in the next step without purification. ¹H NMR (400 MHz, CDCl₃) □ ppm 1.43-1.48 (m, 3H), 4.46-4.52 (m, 2H), 7.65-7.66 (m, 1H), 7.94 (d, 2H).

### Step I4-B: Synthesis of ethyl 3,3-dichloro-2-(3,5-dichlorophenyl)prop-2-enoate

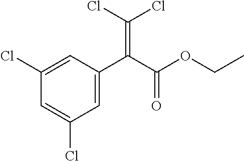 [SMILES: CCOC(=O)C(=C(Cl)Cl)c1cc(Cl)cc(Cl)c1]

To a stirred solution of ethyl 2-(3,5-dichlorophenyl)-2-oxo-acetate (90.0%, 18.0 g, 65.6 mmol) in carbon tetrachloride (110 mL) was added triphenyl phosphine (34.4 g, 131 mmol) at room temperature and stirred at 95° C. for 2h. The solvent was evaporated and the residue was diluted with water (50 mL) and extracted twice with ethyl acetate (2×50 mL). The combined organic layers were dried over sodium sulfate and concentrated under reduced pressure. The residue was purified by chromatography (combiflash, 2.5 to 5% ethyl acetate in Hexane) to afford 17.1 g of ethyl 3,3-dichloro-2-(3,5-dichlorophenyl)prop-2-enoate as a light yellow oil. ¹H NMR (400 MHz, CDCl3) δ ppm 7.4 (s, 1H), 7.28 (m, 2H), 4.30 (m, 2H), 1.30 (m, 3H).

### Step I4-B: Synthesis of 3,3-dichloro-2-(3,5-dichlorophenyl)prop-2-enoic acid

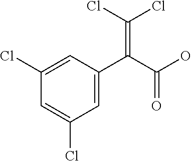 [SMILES: O=C(O)C(=C(Cl)Cl)c1cc(Cl)cc(Cl)c1]

To a stirred solution of ethyl 3,3-dichloro-2-(3,5-dichlorophenyl)prop-2-enoate (6.00 g, 19.1 mmol) in methanol (30.0 mL) was added 1M potassium hydroxide (1.32 M, 30.0 mL, 39.7 mmol) in water (30.0 mL) at 0° C. and stirred at rt for 16h. The solvent was evaporated and the residue was acidified by 2M hydrogen chloride and extracted twice with ethyl acetate (2×200 mL). The combined organic layers were dried over Na2SO4 and concentrated under reduced pressure. The residue was purified by chromatography (combiflash, 17 to 25% ethyl acetate in Hexane) to afford 2.85 g (63.2%) of 3,3-dichloro-2-(3,5-dichlorophenyl)prop-2-enoic acid. ¹H NMR (400 MHz, CDCl3) δ ppm 7.76 (s, 1H), 7.52 (m, 2H).

### Example I5: Preparation of 4-(2-cyanoethyl)-6-iodo-1-methyl-7-oxo-pyrazolo[1,5-a]pyrimidin-4-ium-5-olate

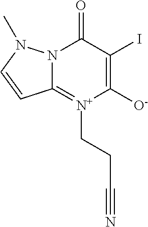 [SMILES: Cn1ccc2n1c(=O)c(I)c([O-])[n+]2CCC#N]

A 25 mL flask under argon is charged with 3-[(1-methylpyrazol-3-yl)amino]propanenitrile (1 g, 6.66 mmol), dichloromethane (20 mL), malonic acid (0.69 g, 6.66 mmol) and DCC (1.85 g, 14.6 mmol). The reaction mixture is stirred at room temperature for two hours and N-iodosuccinimide (1.79 g, 7.97 mmol) is added as a solid. The brown mixture is further stirred at room temperature for two hours and the resulting suspension is diluted with methanol and filtered to give 4-(2-cyanoethyl)-6-iodo-1-methyl-7-oxo-pyrazolo[1,5-a]pyrimidin-4-ium-5-olate as a white solid (1.92 g). ¹H-NMR (400 MHz, DMSO-d6): δ 8.38 (d, 1H), 6.75 (d, 1H), 4.26 (s, 3H), 4.24 (m, 2H), 2.86 (m, 2H).

### Example I6: Preparation of 3-[(1-methylpyrazol-3-yl)amino]propanenitrile

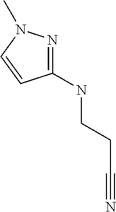

To a solution of 3-amino-1-methylpyrazole (5 mmol, 485 mg) in acetonitrile (5 ml) was added cupric acetate monohydrate (5 mmol, 1 g) at 25° C., then the reaction was stirred at 90° C. for 1h. The reaction was filtrated and concentrated, purified by column chromatography (EtOAc as the eluent) to afford the title compound (247 mg): ¹H NMR (400 MHz, DMSO-d6) δ 7.29 (s, 1H), 5.45 (s, 1H), 5.41 (s, 1H), 3.58 (s, 3H), 3.27-3.19 (m, 2H), 2.68 (m, 2H).

### Example I7: Preparation of 2-methyl-3-[(1-methylpyrazol-3-yl)amino]propanenitrile

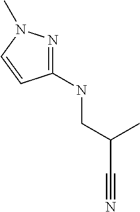

To a solution of 3-Bromo-1-methyl-1H-pyrazole (CAS 151049-87-5, 0.200 g, 1.22 mmol) in 1,4-Dioxane (4 mL) was added, at room temperature, 3-Amino-2-methylpropanenitrile (CAS 96-16-2, 0.108 g, 1.22 mmol) and sodium tert-butoxide (0.422 g, 4.26 mmol). The resulting beige suspension was degassed under Argon for 2 min, then BrettPhos Pd G3 (0.0552 g, 0.0609 mmol) was added and the suspension was stirred at 80° C. for 21h. Then temperature was allowed to come back at room temperature. The reaction medium was filtered through a sintered disc filter funnel. The solid was washed with dichloromethane (×3). The filtrate was concentrated under reduced pressure at 40° C. The crude was purified by Combiflash chromatography (4 g cartridge) with a gradient of cyclohexane/ethyl acetate to give the title compound (0.0212 g). ¹H NMR (400 MHz, CDCl3-d) δ ppm 7.12 (s, 1H), 5.7 (s, 1H), 3.98 (m, 1H), 3.74 (s, 3H), 3.40 (m, 2H), 3.12 (m, 1H).

### b) Synthesis of Final Compounds:

### Example P1: Preparation of 4-[(2-chlorothiazol-5-yl)methyl]-6-(3,5-dichlorophenyl)-1-methyl-7-oxo-pyrazolo[1,5-a]pyrimidin-4-ium-5-olate

### Methode A:

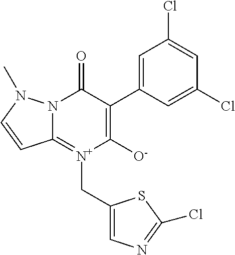 [SMILES: Cn1ccc2n1c(=O)c(-c1cc(Cl)cc(Cl)c1)c([O-])[n+]2Cc1cnc(Cl)s1]

A 250 ml flask was charged with N-[(2-chlorothiazol-5-yl)methyl]-1-methyl-pyrazol-3-amine (4.00 g, 17.5 mmol), bis(2,4,6-trichlorophenyl) 2-(3,5-dichlorophenyl)propanedioate (11.2 g, 18.4 mmol, 1.05) and toluene (120 mL). The resulting solution was stirred 3 hours at 90° C. After cooling, the suspension was diluted with methyl-tert-butylether, filtered and rinsed with methyl-tert-butylether to give the tittle compound (6.59 g, 85.3% Yield). ¹H NMR (400 MHz, DMSO-d6) δ ppm 4.31 (s, 3H), 5.31 (s, 2H), 6.90 (d, 1H), 7.30 (m, 1H), 7.85 (d, 2H), 7.92 (s, 1H), 8.48 (d, 1H).

### Methode B:

### Step P1-A: Synthesis of 3,3-dichloro-N-[(2-chlorothiazol-5-yl)methyl]-2-(3,5-dichlorophenyl)-N-(1-methylpyrazol-3-yl)prop-2-enamide

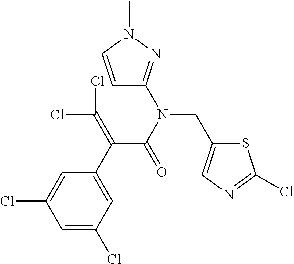 [SMILES: Cn1ccc(N(Cc2cnc(Cl)s2)C(=O)C(=C(Cl)Cl)c2cc(Cl)cc(Cl)c2)n1]

A 25 ml flask was charged with 3,3-dichloro-2-(3,5-dichlorophenyl)prop-2-enoic acid (Step I4-B, 0.7178 g, 2.5107 mmol), toluene (5.71 mL) and thionyl chloride (0.3684 mL, 5.02 mmol). The mixture was refluxed until end of gassing (45 min). The brown solution concentrated under vacuum and the residue was dissolved in tetrahydrofuran (4.5649 mL).

In a separate 25 ml flask, was dissolved N-[(2-chlorothiazol-5-yl)methyl]-1-methyl-pyrazol-3-amine 0.522 g, 2.2825 mmol) in tetrahydrofuran (4.56 mL). The solution was cooled to 0° C. and isopropylmagnesium chloride (2.0 mol/L in diethyl ether, 1.3 mL, 2.5107 mmol) was added dropwise. The solution was stirred for an hour. Then the solution of acid chloride was then slowly added dropwise at 0° C. The reaction mixture was allowed to reach room temperature over night. The mixture was diluted with water and extracted twice with ethyl acetate. The combined organic layers were dried over sodium sulfate and concentrated under reduced pressure. The residue was purified by flash chromatography to give the title compound (0.9 g, 79.40% Yield). ¹H NMR (400 MHz, DMSO-d6) δ ppm 7.35 (m 3H), 7.0 (s, 2H), 6.0 (s, 1H), 4.8 (s, 2H), 3.84 (s, 3H).

### Step P1-B: Synthesis of 7-chloro-4-[(2-chlorothiazol-5-yl)methyl]-6-(3,5-dichlorophenyl)-1-methyl-pyrazolo[1,5-a]pyrimidin-4-ium-5-one; tetrachloroalumanuide

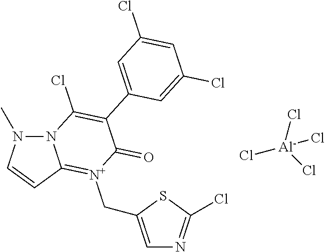

A 250 mL flask was charged with 3,3-dichloro-N-[(2-chlorothiazol-5-y)methyl]-2-(3,5-dichlorophenyl)-N-(1-methylpyrazol-3-yl)prop-2-enamide (3.54 g, 7.13 mmol), 1,2-dichloroethane (53.5 mL) and aluminum chloride (1.14 g, 8.55 mmol). The resulting solution was refluxed over night then allowed to cool down at room temperature. The mixture was diluted with dichloromethane (70 mL) and the solid was filtered to give the tittle compound (2.56 g, 57% Yield). The solid was used for the next step without extra purification. ¹H NMR (400 MHz, DMSO-d6) δ ppm 9.1 (s, 1H), 8.04 (s, 1H), 7.88 (s, 1H), 7.45 (s, 1H), 5.6 (s, 2H), 4.45 (s, 3H).

### Step P1-C: Synthesis of 4-[(2-chlorothiazol-5-yl)methyl]-6-(3,5-dichlorophenyl)-1-methyl-7-oxo-pyrazolo[1,5-a]pyrimidin-4-ium-5-olate

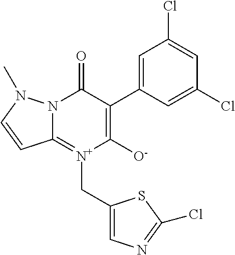 [SMILES: Cn1ccc2n1c(=O)c(-c1cc(Cl)cc(Cl)c1)c([O-])[n+]2Cc1cnc(Cl)s1]

A 25 mL flask was charged with 7-chloro-4-[(2-chlorothiazol-5-yl)methyl]-6-(3,5-dichlorophenyl)-1-methyl-pyrazolo[1,5-a]pyrimidin-4-ium-5-one; tetrachloroaluminate (Step P1-B, 0.1 g, 0.1587 mmol), tetrahydrofuran (2 mL) and water (1 mL). The reaction was stirred 6 hours at reflux, then at room temperature for the weekend. 3 ml of water was added and the mixture was reflux 30 hours. The mixture was diluted with water and extracted twice with dichloromethane and one time with ethyl acetate. The combined organic layers were dried over sodium sulfate and concentrated under reduced pressure to give the title compound (0.0500 g, 71.3% Yield). ¹H NMR (400 MHz, DMSO-d6) δ ppm 4.31 (s, 3H), 5.31 (s, 2H), 6.90 (d, 1H), 7.30 (m, 1H), 7.85 (d, 2H), 7.92 (s, 1H), 8.48 (d, 1H).

### Example P2: Preparation of 6-(4-chloro-4,4-difluoro-butyl)-4-[(2-chlorothiazol-5-yl)methyl]-1-methyl-7-oxo-pyrazolo[1,5-a]pyrimidin-4-ium-5-olate

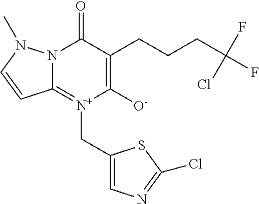 [SMILES: Cn1ccc2n1c(=O)c(CCCC(F)(F)Cl)c([O-])[n+]2Cc1cnc(Cl)s1]

In a flask containing disodium 2-(4-chloro-4,4-difluoro-butyl)propanedioate (Obtained from hydrolysis of compound available (CAS: 168901-97-1), 0.900 g) was added dichloromethane (7.50 mL), oxalyl dichloride (0.636 mL) and one drop of DMF. The mixture was stirred for 3 hours. N-[(2-chlorothiazol-5-yl)methyl]-1-methyl-pyrazol-3-amine (B, 0.150 g, 0.656 mmol, 1.00) was added and the reaction was stirred over night at RT. After completion of the reaction mixture was diluted with water and the aqueuous layer was additionned of a saturated solution of sodium bicarbonate until pH 6-7. The aqueuous layer extracted with dichloromethane (2×) and the combinated organic layers were washed with brine, dried over sodium sulphate and concentrated. The crude was purified by silica gel column chromatography (3RF 200) to give the title compound (0.109 g, 39.3% Yield). ¹H NMR (400 MHz, DMSO-d6) δ ppm 1.63-1.77 (m, 2H), 2.34-2.46 (m, 4H), 4.28 (s, 3H), 5.25 (s, 2H), 6.80 (d, 1H), 7.86 (s, 1H), 8.34 (d, 1H).

### Example P3: Preparation of 4-(2-cyanoethyl)-1-methyl-7-oxo-6-[3-(trifluoromethyl)phenyl]pyrazolo[1,5-a]pyrimidin-4-ium-5-olate

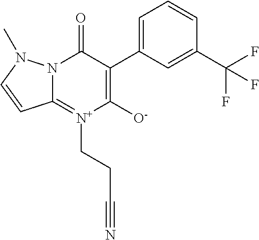 [SMILES: Cn1ccc2n1c(=O)c(-c1cccc(C(F)(F)F)c1)c([O-])[n+]2CCC#N]

A 5 mL flask is charged with 4-(2-cyanoethyl)-6-iodo-1-methyl-7-oxo-pyrazolo[1,5-a]pyrimidin-4-ium-5-olate (100 mg, 0.29 mmol), N-methyl-2-pyrrolidone (2.9 mL), potassium fluoride (152 mg, 2.62 mmol), [3-(trifluoromethyl)phenyl]boronic acid (221 mg, 1.16 mmol) and the pre-catalyst [P(tBu)₃]Pd(crotyl)Cl (5.8 mg, 0.0145 mmol). The yellow suspension is stirred at 80° C. for four hours, cooled at room temperature, filtered on Celite and concentrated. Purification by reverse phase chromatography on a C₁₈ column and eluting with (water/acetonitrile, gradient 100:0->0:100) afford 4-(2-cyanoethyl)-1-methyl-7-oxo-6-[3-(trifluoromethyl)phenyl]pyrazolo[1,5-a]pyrimidin-4-ium-5-olate as a white solid (6 mg). ¹H-NMR (400 MHz, CDCl₃): δ 7.99 (s, 1H), 7.94-7.82 (m, 1H), 7.62 (d, 1H), 7.53-7.39 (m, 2H), 6.27 (d, 1H), 4.49 (s, 3H), 4.28 (m, 2H), 2.95 (m, 3H).

### Example P3: Preparation of 4-(2-cyanoethyl)-1-methyl-7-oxo-6-(2,2,2-trifluoroacetyl)pyrazolo[1,5-a]pyrimidin-4-ium-5-olate

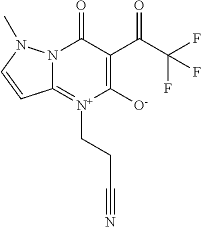 [SMILES: Cn1ccc2n1c(=O)c(C(=O)C(F)(F)F)c([O-])[n+]2CCC#N]

In a 25 mL flask containing 3-[(1-methylpyrazol-3-yl)amino]propanenitrile (Example 16, 0.1 g) and malonic acid (0.069 g, 1 eq.) was added N,N′-diisopropylmethanediimine (0.181 g, 2.2 eq.). The yellow suspension was stirred at room temperature for 1h, then (2,2,2-trifluoroacetyl) 2,2,2-trifluoroacetate (0.167 g, 1.2 eq.) was added. The mixture was stirred at room temperature for 2h. The suspension was filtered and spilled with dichloromethane, then the liquid was concentrated under vacuum. The crude was purified by silica gel column chromatography (RF 200, cyclohexane/ethyl acetate to dichloromethane/MeOH) to give the title compound (0.117 g). ¹H-NMR (400 MHz, DMSO-d6): δ 8.52 (s, 1H), 6.78 (m, 1H), 4.18 (m, 2H), 4.85 (m, 2H).

### Example P4: Preparation of 4-(2-chloroethyl)-6-(3,5-dichlorophenyl)-1-methyl-7-oxo-pyrazolo[1,5-a]pyrimidin-4-ium-5-olate B20

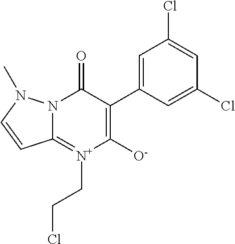 [SMILES: Cn1ccc2n1c(=O)c(-c1cc(Cl)cc(Cl)c1)c([O-])[n+]2CCCl]

### Step P4-A: Synthesis of 6-(3,5-dichlorophenyl)-5-hydroxy-1-methyl-pyrazolo[1,5-a]pyrimidin-7-one

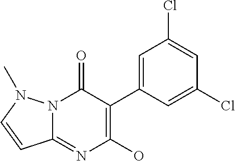 [SMILES: Cn1ccc2nc(O)c(-c3cc(Cl)cc(Cl)c3)c(=O)n21]

A flask containing 1-methylpyrazol-3-amine (1.0 g, 10.30 mmol), bis(2,4,6-trichlorophenyl) 2-(3,5-dichlorophenyl)propanedioate (7.511 g, 12.36 mmol) in toluene (50 mL) was stirred 1 hours under reflux. After cooling, suspension was diluted with TBME, filtered and the solid was rinsed with TBME to give 6-(3,5-dichlorophenyl)-5-hydroxy-1-methyl-pyrazolo[1,5-a]pyrimidin-7-one (3.0 g, 94% Yield). LC-MS (method A): 310 (M+1)⁺, retention time 0.73 min.

### Step P4-B: Synthesis of 4-(2-chloroethyl)-6-(3,5-dichlorophenyl)-1-methyl-7-oxo-pyrazole [1,5-a]pyrimidin-4-ium-5-olate B20

A flask containing 6-(3,5-dichlorophenyl)-5-hydroxy-1-methyl-pyrazolo[1,5-a]pyrimidin-7-one (0.5 g, 1.612 mmol), N,N-dimethylformamide (10 mL), potassium carbonate (0.2674 g, 1.94 mmol) and lithium bromide (0.1400 g, 1.61 mmol). was stirred 10 minutes at room temperature. Then, 1-bromo-2-chloroethane (0.685 mL, 8.06 mmol) was added. The white suspension was stirred for 5 hours at room temperature. After completion of the reaction mixture was diluted with water and the aqueuous layer extracted with ethyl acetate (3×) and the combinated organic layers were washed with brine, dried over sodium sulphate and concentrated. the white solid was suspended in Aceton and filtered. The filtrate was concentrated under vacuum and purified by silica gel column chromatography (3RF 200) to give the title compound (0.058 g, 0.156 mmol, 9.7% Yield −90/92% purity). LC-MS (method A): 372 (M+1)+, retention time 0.92 min. The mixture was used without extra purification.

### Example P5: Preparation of 4-(2-chloroethyl)-6-(3,5-dichlorophenyl)-1-methyl-7-oxo-pyrazolo[1,5-a]pyrimidin-4-ium-5-olate B21

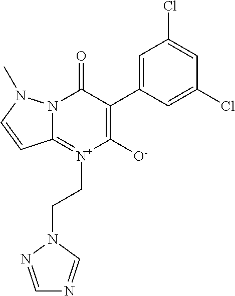 [SMILES: Cn1ccc2n1c(=O)c(-c1cc(Cl)cc(Cl)c1)c([O-])[n+]2CCn1cncn1]

To a solution of 4-(2-chloroethyl)-6-(3,5-dichlorophenyl)-1-methyl-7-oxo-pyrazole [1,5-a]pyrimidin-4-ium-5-olate B20 (0.05 g, 0.1342 mmol, 90% purity) in N,N-dimethylformamide (0.52 mL) was added 1H-1,2,4-triazole (0.04634 g, 0.6710 mmol). The mixture was stirred at 50° C. for 1h30. The mixture was then diluted in water and ethyl acetate, then after separation of the organic phase, the aqueous. layer was then extracted 3 times with ethyl acetate. The combinated organic layers were washed with brine, dried over sodium sulphate and concentrated. The crude was purified by chromatography using a dichloromethane and methanol gradient to afford the title compound (10 mg, 18.4% yield). LC-MS (method A): 405 (M+1)+, retention time 0.75 min.

### Example P6: Preparation of 4-[1-(2-chlorothiazol-5-yl)ethyl]-6-(3,5-dichlorophenyl)-1-methyl-7-oxo-pyrazolo[1,5-a]pyrimidin-4-ium-5-olate A61

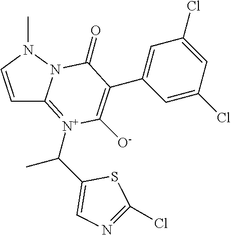 [SMILES: CC(c1cnc(Cl)s1)[n+]1c([O-])c(-c2cc(Cl)cc(Cl)c2)c(=O)n2c1ccn2C]

### Step P6-A: Synthesis of N-[1-(2-chlorothiazol-5-yl)ethyl]-1-methyl-pyrazol-3-amine

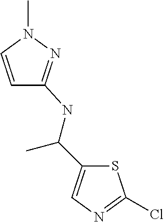

To a solution of 1-methylpyrazol-3-amine (0.2 g, 2.059 mmol) in ethanol (13 mL, 226.5 mmol),2-chlorothiazole-5-carbaldehyde (0.4559 g, 3.089 mmol), molecular sieve 4A (1.8 g) and acetic acid (0.354 mL, 6.18 mmol) were added. The mixture was stirred at 78° C. for 1h. After completion of the reaction, the reaction mixture was filtered through celite, the solvent was evaporated under reduced pressure. The residue was dissolved in tetrahydrofuran (25 mL, 308.9 mmol), and under cooling with ice, bromo(methyl)magnesium (8.24 mL, 8.237 mmol) was added drop-wise. After completion of the drop-wise addition, the mixture was stirred at room temperature for 3h. The mixed solution was cooled with ice, and water was added. The solution was extracted twice with ethyl acetate. Then, the organic layer was washed with brine and dried over anhydrous sodium sulfate and concentrated. The residue was purified by chromatography using a cyclohexane/ethyl acetate gradient to afford the title compound. LC-MS (method A): 243 (M+1)+, retention time 0.73 min.

### Step P6-B: Synthesis of 4-[1-(2-chlorothiazol-5-yl)ethyl]-6-(3,5-dichlorophenyl)-1-methyl-7-oxo-pyrazolo[1,5-a]pyrimidin-4-ium-5-olate A61

The synthesis was done using the analogue condition use for Example P1, Method A. LC-MS (method A): 455 (M+1)⁺, retention time 1.03 min.

Alternative synthesis of mesoionics to the ones described in, for example in WO09099929, WO16171053 or WO11017342.

### Example P7: Preparation of 1-[(2-chlorothiazol-5-yl)methyl]-3-(3,5-dichlorophenyl)-4-oxo-pyrido[1,2-a]pyrimidin-1-ium-2-olate

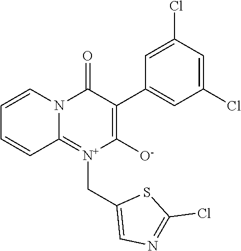 [SMILES: O=c1c(-c2cc(Cl)cc(Cl)c2)c([O-])[n+](Cc2cnc(Cl)s2)c2ccccn12]

### Synthesis A: Preparation of 1-[(2-chlorothiazol-5-yl)methyl]-3-(3,5-dichlorophenyl)-4-oxo-pyrido[1,2-a]pyrimidin-1-ium-2-olate

### Step 1: Preparation of 3,3-dichloro-N-[(2-chlorothiazol-5-yl)methyl]-2-(3,5-dichlorophenyl)-N-(2-pyridyl)prop-2-enamide

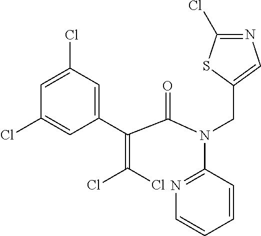 [SMILES: O=C(C(=C(Cl)Cl)c1cc(Cl)cc(Cl)c1)N(Cc1cnc(Cl)s1)c1ccccn1]

To a 25 mL RBF under argon were added N-[(2-Chloro-5-thiazolyl)methyl]-2-pyridinamine (CAS 1176959-68-4 or prepared as described in WO09099929, 0.565 g) and tetrahydrofuran (4.76 mL). The solution was cool down to 0° C., then isopropyl magnesium chloride (1.10 equiv., 1.28 g, 1.31 mL) was added dropwise and the reaction mixture was stirred at room temperature. The reaction mixture was cooled down to 0° C. Then 3,3-dichloro-2-(3,5-dichlorophenyl) prop-2-enoyl chloride (Prepared by analogy with step P1-A, 0.7239 g) in tetrahydrofuran (4.7563 mL) was added dropwise. The reaction mixture was stirred at room temperature overnight. The reaction mixture was directly concentrated under reduced pressure and purified by flash column chromatography (Silica, 25 g; cyclohexane to cyclohexane/ethyl acetate 85:15) to afford 0.94 g of the title compound (70% yield). 1H NMR (400 MHz, DMSO-d₆) δ ppm 5.10-5.28 (m, 2H) 6.91-7.17 (m, 2H) 7.29-7.45 (m, 2H) 7.56-7.69 (m, 2H) 7.92-8.01 (m, 1H) 8.34-8.48 (m, 1H).

### Step 2: Preparation of 1-[(2-chlorothiazol-5-yl)methyl]-3-(3,5-dichlorophenyl)-4-oxo-pyrido[1,2-a]pyrimidin-1-ium-2-olate

To a 25 mL flask under argon were added 3,3-dichloro-N-[(2-chlorothiazol-5-yl)methyl]-2-(3,5-dichlorophenyl)-N-(2-pyridyl)prop-2-enamide (0.087 g) was dissolved in 1,2-dichloroethane (3.07 mL), then aluminum chloride (1 equiv., 0.0205 g) was added. The solution was heated to 85° C. four hours. The reaction was allowed to cool down to room temperature. The reaction mixture was allowed to cool down to room temperature, then it was diluted with dichloromethane and aqueous sodium bicarbonate solution. After separation of the layer, the aqueous layer was extracted twice with dichloromethane. The combined organic layer was washed once with brine, dried over magnesium sulfate, filtered and concentrated under reduced pressure to give the title compound (75 mg, 93% yield). ¹H NMR (400 MHz, DMSO-d₆) □ ppm 5.64 (s, 2H), 7.39 (s, 1H), 7.58 (t, 1H), 7.94 (m, 2H), 8.01 (s, 1H), 8.17 (d, 1H), 8.40 (t, 1H), 9.31 (d, 1H). The analytical data were identical to compounds coming from Synthesis B and C.

The 4-chloro-1-[(2-chlorothiazol-5-yl)methyl]-3-(3,5-dichlorophenyl)pyrido[1,2-a]pyrimidin-5-ium-2-one; tetrachloroalumanuide, the intermediate of the preview reaction could be prepared and isolated it.

### Preparation of 4-chloro-1-[(2-chlorothiazol-5-yl)methyl]-3-(3,5-dichlorophenyl)pyrido[1,2-a]pyrimidin-5-ium-2-one; tetrachloroalumanuide

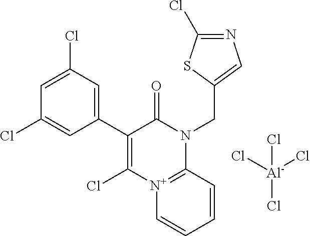

In a 20 mL vial, the 3,3-dichloro-N-[(2-chlorothiazol-5-yl)methyl]-2-(3,5-dichlorophenyl)-N-(2-pyridyl)prop-2-enamide (110 mg, 0.11 g) was dissolved in 1,2-dichloroethane (2.44 g, 1.94 mL), then aluminum chloride (1 equiv., 0.02585 g) was added. The solution was heated to 85° C. The reaction was allowed to come back at room temperature and filtrate to give the title compound (116 mg, 86% yield). ¹H NMR (400 MHz, DMSO-d₆) □ ppm 5.89 (s, 2H), 7.51 (d, 2H), 7.88-7.94 (m, 1H), 7.95-8.01 (m, 1H), 8.03-8.08 (m, 1H), 8.64 (s, 1H), 8.79 (s, 1H), 9.57 (d, 1H).

### Synthesis B: Preparation of 1-[(2-chlorothiazol-5-yl)methyl]-3-(3,5-dichlorophenyl)-4-oxo-pyrido[1,2-a]pyrimidin-1-ium-2-olate (classical method by analogy with methods described in WO09099929, WO16171053 or WO11017342)

In a 5 mL vial, N-[(2-Chloro-5-thiazolyl)methyl]-2-pyridinamine (CAS 1176959-68-4 or prepared as described in WO09099929,1 equiv., 0.05932 g) was dissolved in 1,2-dichloroethane (1.31 mL). Then n,n-diisopropylethylamine (3 equiv., 0.14 mL) was added. The solution was cooled down to 0° C. In a separate flask, 3,3-dichloro-2-(3,5-dichlorophenyl)prop-2-enoyl chloride (Prepared by analogy with step P1-A, 2.114 mmol, 0.08 g) was dissolved in 1,2-dichloroethane (1.31 mL) and added dropwise to the prior pale yellow solution at 0° C. The reaction mixture was allowed to warm up to room temperature and stirred at room temperature over the week end. Water and ethyl acetate were added to the reaction mixture. After separation of the layers, the aqueous layer was extracted twice with ethyl acetate. The combined organic layer was washed once with brine, dried over magnesium sulfate, filtered and concentrated under reduced pressure to give 1-[(2-chlorothiazol-5-yl)methyl]-3-(3,5-dichlorophenyl)-4-oxo-pyrido[1,2-a]pyrimidin-1-ium-2-olate (153 mg, 65% yield). The analytical data were identical to compound coming from Synthesis A and C.

### Synthesis C: Preparation of 1-[(2-chlorothiazol-5-yl)methyl]-3-(3,5-dichlorophenyl)-4-oxo-pyrido[1,2-a]pyrimidin-1-ium-2-olate

N-[(2-Chloro-5-thiazolyl)methyl]-2-pyridinamine (CAS 1176959-68-4 or prepared as described in WO09099929,1 equiv., 0.059 g) was dissolved in 1,2-dichloroethane (5 mL/mmol, 1.650 g, 1.314 mL). Then N,N-diisopropylethylamine (3 equiv., 0.100 g, 0.14 mL) was added. The solution was cooled down to 0° C. In a separate flask, 3,3-dichloro-2-(3,5-dichlorophenyl) prop-2-enoyl chloride (Prepared by analogy with step P1-A, 0.7239 g) in 1,2-dichloroethane (1.314 mL) and added dropwise to the prior pale yellow solution at 0° C. The reaction mixture was still stirred at 0° C. for 2h, then overweekend at room temperature. A mixture of water and ethyl acetate were added to the reaction. After separation of the layers, the aqueous layer was extracted twice with ethyl acetate. The combined organic layer was washed once with brine, dried over magnesium sulfate, filtered and concentrated under reduced pressure to give the title compound. The analytical data were identical to compound coming from Synthesis A and B.

The 2-chloro-1-[(2-chlorothiazol-5-yl)methyl]-3-(3,5-dichlorophenyl)pyrido[1,2-a]pyrimidin-1-ium-4-one; chloride, the intermediate of the preview reaction could be prepared and isolated it.

### Preparation of 2-chloro-1-[(2-chlorothiazol-5-yl)methyl]-3-(3,5-dichlorophenyl)pyrido[1,2-a]pyrimidin-1-ium-4-one: chloride

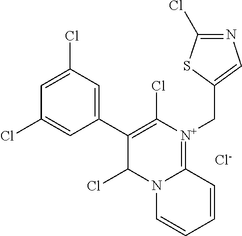 [SMILES: ClC1=C(c2cc(Cl)cc(Cl)c2)C(Cl)N2C=CC=CC2=[N+]1Cc1cnc(Cl)s1 ; [Cl-]]

In a 25 mL flask, N-[(2-Chloro-5-thiazolyl)methyl]-2-pyridinamine (CAS 1176959-68-4 or prepared as described in WO09099929,1 equiv., 0.502 g) was dissolved in 1,2-dichloroethane (5.29 mL). Then n,n-diisopropylethylamine (3 equiv., 1.13 mL) was added. The solution was cooled down to 0° C. In a separate flask, 3,3-dichloro-2-(3,5-dichlorophenyl)prop-2-enoyl chloride (Prepared by analogy with step P1-A, 2.114 mmol, 0.6435 g) was dissolved in 1,2-dichloroethane (6.69 g, 5.29 mL) and added dropwise to the prior pale yellow solution at 0° C. The reaction mixture was allowed to warm up to room temperature. The reaction mixture was concentrated under reduced pressure. Then it was diluted with diethyl ether and the precipitate was filtered off and washed 5 times with diethyl ether to give the title compound. ¹H NMR (400 MHz, DMSO-d₆) □ ppm 6.30 (s, 2H), 7.5 (s, 2H), 7.85 (s, 1H), 7.90 (s, 1H), 8.20 (t, 1H), 8.70 (d, 1H), 8.85 (t, 1H), 9.15 (d, 1H). LC-MS (method A): 456 (M+1)*, retention time 0.82 min.

The compounds according to the following Table A, A2 and B below can be prepared according to the methods described above, commercially available or by known reactions from literature. The examples which follow are intended to illustrate the invention and show preferred compounds of formula I. Intermediates not included in the tabled C, D and E were used without any purification.

Table A: This table discloses compounds of the formula (1) wherein R₂ and R₃ are hydrogen and V and W are oxygen, R_{1b}, R₂ and R₃ are each H:

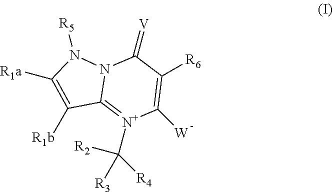

| Comp. | | | | | |
|---|---|---|---|---|---|
| No. | R₁ₐ | R₄ | R₅ | R₆ | Analytical data |
| A1 | H | 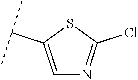 | CH₃ | 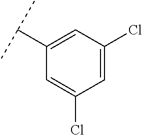 | See experimental part (Example P1) |
| A2 | H | 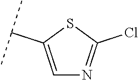 | CH₃ | 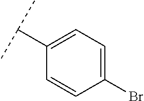 | ¹H NMR (400 MHz, DMSO-d₆) δ ppm 4.29 (s, 3 H), 5.29 (s, 2 H), 6.87 (d, 1 H), 7.44 (d, 2 H), 7.63 (d, 2 H), 7.90 (s, 1 H), 8.43 (d, 1 H). |
| A3 | H | 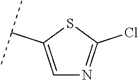 | CH₃ | 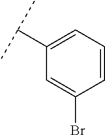 | LC-MS (method A): 453 (M + 1)⁺, retention time 0.86 min |
| A4 | H | 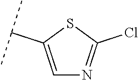 | CH₃ | 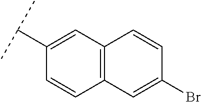 | ¹H NMR (600 MHz, DMSO-d6) δ ppm 4.31 (s, 3 H), 5.32 (s, 2 H), 6.89 (d, 1 H), 7.54 (m, 1 H) 7.77 (d, 1 H) 7.80 (d, 1 H) 7.87 (m, 1 H), 7.92 (s, 1 H), 8.10 (d,1 H), 8.15 (s, 1 H), 8.45 (d,1 H) |
| A5 | H | 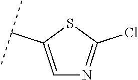 | CH₃ | CH₂CH₂CH₂CF₂Cl | See experimental part (Example P2) |
| A6 | H | 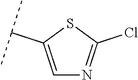 | CH₃ | 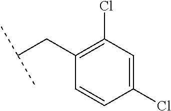 | LC-MS (method A): 457 (M + 1)⁺, retention time 0.94 min |
| A7 | H | 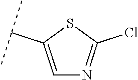 | CH₃ | 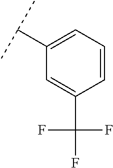 | LC-MS (method A): 441 (M + 1)⁺, retention time 0.93 min |
| A8 | H | 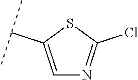 | CH₃ | 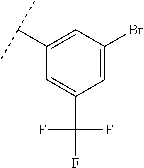 | LC-MS (method A): 521 (M + 1)⁺, retention time 1.06 min |
| A9 | H | 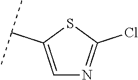 | cyclo- prop- yl | 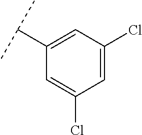 | LC-MS (method A): 469(M + 1)⁺, retention time 1.02 min |
| A10 | H | 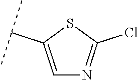 | CHF₂ | 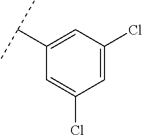 | LC-MS (method A): 479(M + 1)⁺, retention time 1.08 min |
| A11 | H | 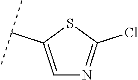 | Ph | 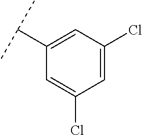 | LC-MS (method A): 505(M + 1)⁺, retention time 1.08 min |
| A12 | H | 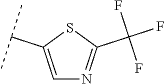 | CH₃ | 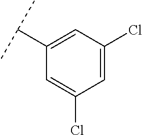 | LC-MS (method A): 474(M + 1)⁺, retention time 1.05 min |
| A13 | H | 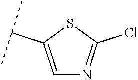 | CH₂ CH₃ | 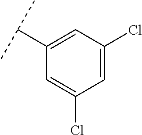 | LC-MS (method A): 457(M + 1)⁺, retention time 1.04 min |
| A14 | H | 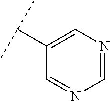 | CH₃ | 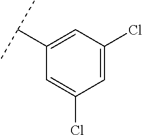 | LC-MS (method A): 402(M + 1)⁺, retention time 0.78 min |
| A15 | H | 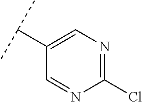 | CH₃ | 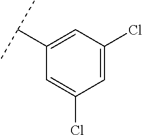 | LC-MS (method A): 437 (M + 1)⁺, retention time 0.92 min |
| A16 | H | 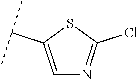 | iso- prop- yl | 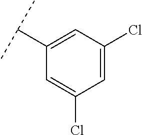 | LC-MS (method A): 471 (M + 1)⁺, retention time 1.10 min |
| A17 | H | 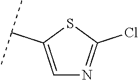 | CH₂CF₃ | 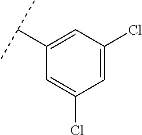 | LC-MS (method A): 511(M + 1)⁺, retention time 1.10 min |
| A18 | CH₃ | 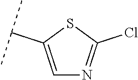 | CH₃ | 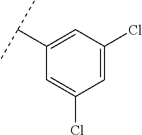 | LC-MS (method A): 457(M + 1)⁺, retention time 1.01 min |
| A19 | CH₃ | 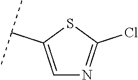 | CH₃ | 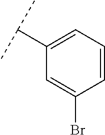 | ¹H NMR (400 MHz, DMSO-d6) δ ppm 2.45 (s, 3 H), 4.19 (s, 3 H), 5.26 (s, 2 H), 6.77 (s, 1 H), 7.19-7.32 (m, 2 H), 7.69 (m, 1 H), 7.85 (m, 1 H), 7.90 (s, 1 H) |
| A20 | H | 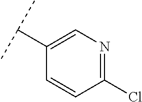 | CH₃ | 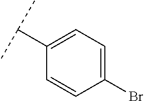 | ¹H NMR (400 MHz, Solvent) δ ppm 4.32 (s, 3 H), 5.23 (s, 2 H), 6.53 (d, 1 H), 7.32 (m, 1 H), 7.39-7.45 (m, 2 H), 7.47 (d, 1 H), 7.60-7.68 (m, 2 H), 8.33-8.40 (m, 2H). |
| A21 | H | 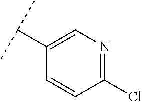 | CH₃ | 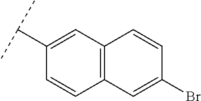 | LC-MS (method A): 497(M + 1)⁺, retention time 0.94 min |
| A22 | H | 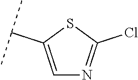 | CH₃ | 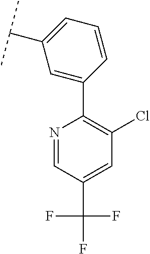 | LC-MS (method A): 552(M + 1)⁺, retention time 1.05 min |
| A23 | H | 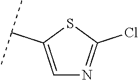 | CH₃ | 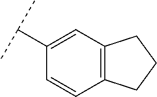 | LC-MS (method A): 413 (M + 1)+, retention time 0.84 min |
| A24 | H | 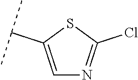 | CH₃ | 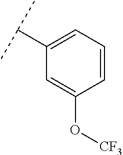 | LC-MS (method A): 457 (M + 1)+, retention time 0.90 min |
| A25 | H | 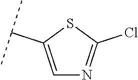 | CH₃CH₂ | 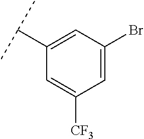 | LC-MS (method A):534 (M + 1)+, retention time 1.07 min |
| A26 | H | 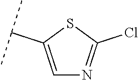 | CH₃ | 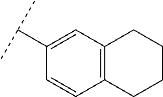 | LC-MS (method A):427 (M + 1)+, retention time 0.90 min |
| A27 | H | 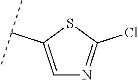 | CH₃CH₂ | 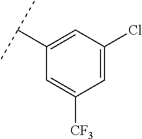 | LC-MS (method A):489 (M + 1)+, retention time 1.07 min |
| A28 | H | 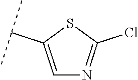 | CH₃ | 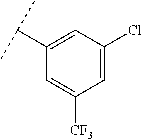 | LC-MS (method A): 475 (M + 1)+, retention time 1.02 min |
| A29 | H | 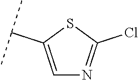 | CH₃ | 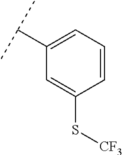 | LC-MS (method A): 473 (M + 1)+, retention time 0.96 min |
| A30 | H | 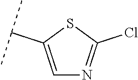 | CH₃ | 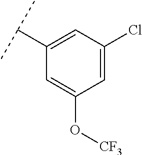 | LC-MS (method A): 491 (M + 1)+, retention time 1.03 min |
| A31 | H | 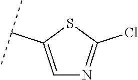 | CH₃CH₂ | 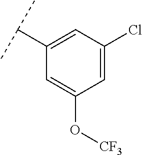 | LC-MS (method A): 505 (M + 1)+, retention time 1.08 min |
| A32 | H | 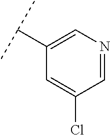 | CH₃ | 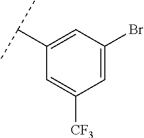 | LC-MS (method A): 513 (M + 1)+, retention time 0.98 min |
| A33 | H | 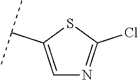 | CH₃ | 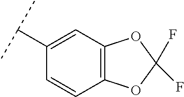 | LC-MS (method A): 453 (M + 1)+, retention time 0.89 min |
| A34 | H | 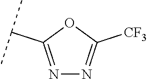 | CH₃ | 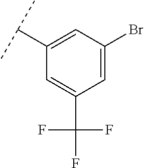 | LC-MS (method A): 539 (M + 1)+, retention time 1.01 min |
| A35 | H | 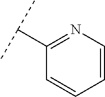 | CH₃ | 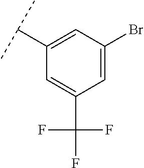 | LC-MS (method A): 478 (M + 1)+, retention time 0.92 min |
| A36 | H | 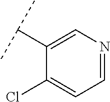 | CH₃ | 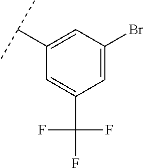 | LC-MS (method A): 513 (M + 1)+, retention time 0.98 min |
| A37 | H | 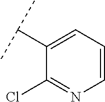 | CH₃ | 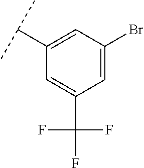 | LC-MS (method A): 513 (M + 1)+, retention time 0.95 min |
| A38 | H | 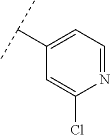 | CH₃ | 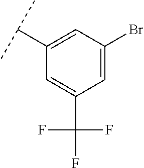 | LC-MS (method A): 513 (M + 1)+, retention time 0.98 min |
| A39 | H | 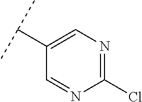 | CH₃ | 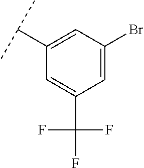 | LC-MS (method A): 514 (M + 1)+, retention time 0.96 min |
| A40 | H | 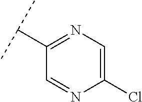 | CH₃ | 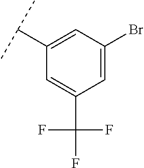 | LC-MS (method A): 516 (M + 1)+, retention time 0.99 min |
| A41 | H | 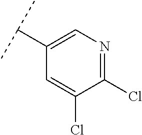 | CH₃ | 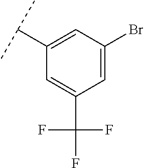 | LC-MS (method A): 548 (M + 1)+, retention time 1.07 min |
| A42 | H | 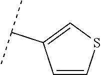 | CH₃ | 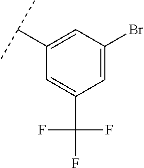 | LC-MS (method A): 485 (M + 1)+, retention time 1.02 min |
| A43 | H | 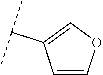 | CH₃ | 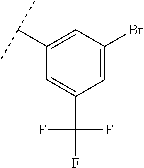 | LC-MS (method A): 468 (M + 1)+, retention time 0.98 min |
| A44 | H | 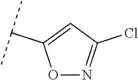 | CH₃ | 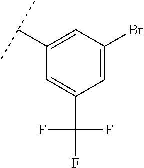 | LC-MS (method A): 503 (M + 1)+, retention time 1.00 min |
| A45 | H | 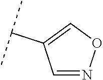 | CH₃ | 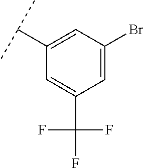 | LC-MS (method A): 469 (M + 1)+, retention time 0.92 min |
| A46 | H | 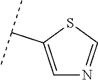 | CH₃ | 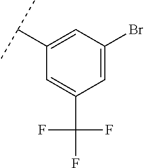 | LC-MS (method A): 485 (M + 1)+, retention time 0.91 min |
| A47 | H | 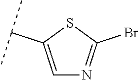 | CH₃ | 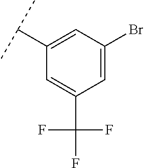 | LC-MS (method A): 563 (M + 1)+, retention time 1.03 min |
| A48 | H | 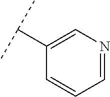 | CH₃ | 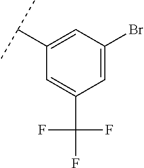 | LC-MS (method A): 479 (M + 1)+, retention time 0.80 min |
| A49 | H | 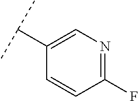 | CH₃ | 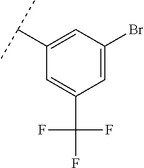 | LC-MS (method A): 497 (M + 1)+, retention time 0.96 min |
| A50 | H | 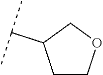 | CH₃ | 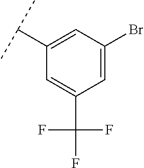 | LC-MS (method A): 472 (M + 1)+, retention time 0.91 min |
| A51 | H | 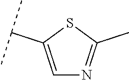 | CH₃ | 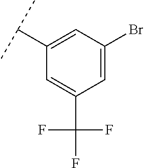 | LC-MS (method A): 499 (M + 1)+, retention time 0.98 min |
| A52 | H | 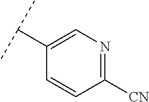 | CH₃ | 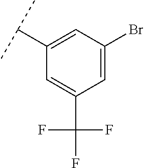 | LC-MS (method A): 504 (M + 1)+, retention time 0.97 min |
| A53 | H | 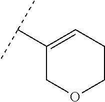 | CH₃ | 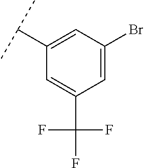 | LC-MS (method A): 484 (M + 1)+, retention time 0.96 min |
| A54 | H | 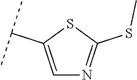 | CH₃ | 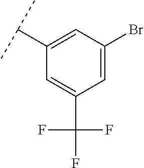 | LC-MS (method A): 533 (M + 1)+, retention time 1.04 min |
| A55 | H | 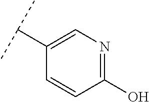 | CH₃ | 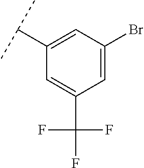 | LC-MS (method A): 495 (M + 1)+, retention time 0.83 min |
| A56 | H | 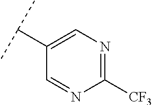 | CH₃ | 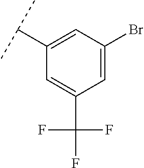 | LC-MS (method A): 548 (M + 1)+, retention time 1.03 min |
| A57 | H | 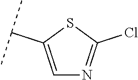 | CH₃CH₂ | 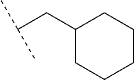 | LC-MS (method A): 407 (M + 1)+, retention time 0.94 min |
| A58 | H | 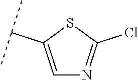 | CH₃CH₂ | 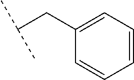 | LC-MS (method A): 401 (M + 1)+, retention time 0.84 min |
| A59 | H | 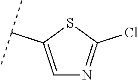 | CH₃ | 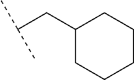 | LC-MS (method A): 393 (M + 1)+, retention time 0.88 min |

Table A2: This table discloses compounds of the formula (1) wherein R₂ and R₃ are hydrogen and V and W are oxygen, R₁ₐ, R_{1b} and R₃ are H. R₅ is methyl:

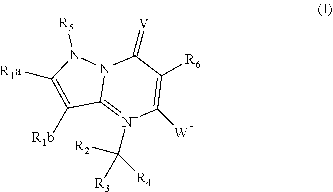

| Comp. | | | | Analytical |
|---|---|---|---|---|
| No. | R₂ | R₄ | R₆ | data |
| A61 | CH₃ | 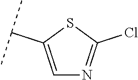 | 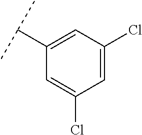 | See example P6 |
| A62 | CH₃ | 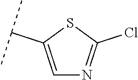 | 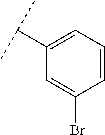 | LC-MS (method A): 467 (M + 1)+, retention time 1.0 min |

Table B: This table discloses compounds of the formula (1) wherein V and Ware oxygen, R₃ and R₄ are H

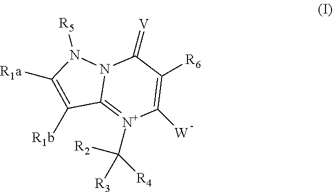

| Comp. | | | | | | |
|---|---|---|---|---|---|---|
| No. | R₁ₐ | R_{1b} | R₂ | R₅ | R₆ | Analytical data |
| B1 | H | H | 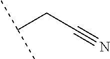 | CH₃ | I | See experimental part (Example I5) |
| B2 | H | H | 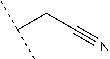 | CH₃ | 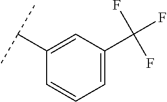 | See experimental part (Example P3) |
| B3 | H | H | 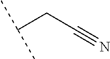 | CH₃ | 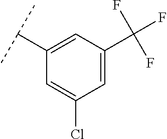 | LC-MS (method A): 397 (M + 1)⁺, retention time 0.87 min |
| B4 | H | H | 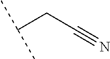 | CH₃ | 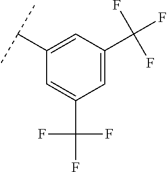 | LC-MS (method A): 431 (M + 1)⁺, retention time 0.92 min |
| B5 | H | H | 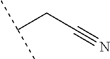 | CH₂ CH₃ | 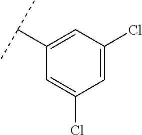 | LC-MS (method A): 377 (M + 1)⁺, retention time 0.88 min |
| B6 | H | H | 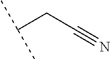 | CH₂ CH₃ | 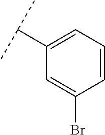 | LC-MS (method A): 387 (M + 1)⁺, retention time 0.77 min |
| B7 | H | H | 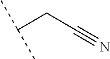 | CH₃ | 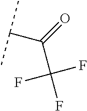 | See example P4 |
| B8 | H | H | 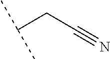 | CH₃ | 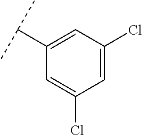 | LC-MS (method A): 363(M + 1)⁺, retention time 0.83 min |
| B9 | H | CH₃ | 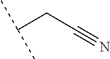 | CH₃ | 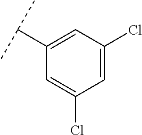 | LC-MS (method A): 377(M + 1)⁺, retention time 0.87 min |
| B10 | H | H | 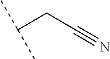 | CH₃ | 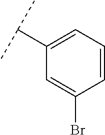 | LC-MS (method A): 375 (M + 1)⁺, retention time 0.71 min |
| B11 | H | H | 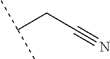 | CH₃ | 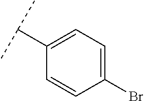 | LC-MS (method A): 375 (M + 1)⁺, retention time 0.69 min |
| B12 | H | H | 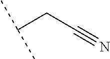 | CH₃ | 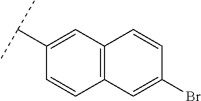 | LC-MS (method A): 425 (M + 1)⁺, retention time 0.82 min |
| B13 | H | H | 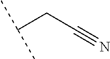 | CH₃ | 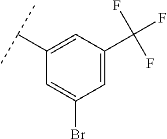 | LC-MS (method A): 443 (M + 1)⁺, retention time 0.90 min |
| B14 | H | H | 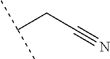 | CH₃ | 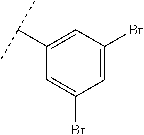 | LC-MS (method A): 453 (M + 1)⁺, retention time 0.85 min |
| B15 | H | H | 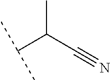 | CH₃ | 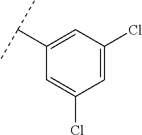 | LC-MS (method A): 421 (M + 1)⁺, retention time 0.86 min |
| B16 | H | H | 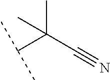 | CH₃ | 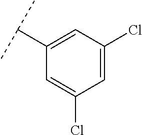 | LC-MS (method A): 391 (M + 1)⁺, retention time 0.89 min |
| B17 | H | H | CF₃ | CH₃ | 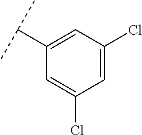 | LC-MS (method A): 392(M + 1)+, retention time 0.97 min |
| B18 | H | H | 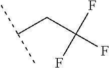 | CH₃ | 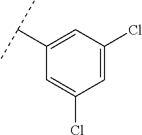 | LC-MS (method A): 406 (M + 1)⁺, retention time 0.94 min |
| B19 | H | H | 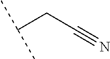 | CH₃ | 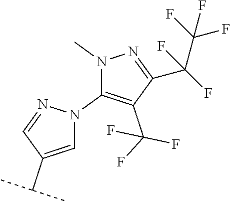 | LC-MS (method A): 551 (M + 1)⁺, retention time 0.95 min |
| B20 | H | H | 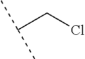 | CH₃ | 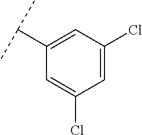 | See example P4 |
| B21 | H | H | 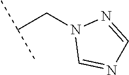 | CH₃ | 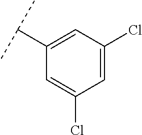 | See example P5 |
| B22 | H | H | 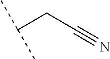 | CH₃ | 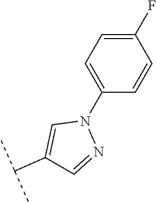 | LC-MS (method A): 379 (M + 1)⁺, retention time 0.78 min |

TABLE C
Examples of intermediates of formula (V), (VI)

| No. | IUPAC name | Structures | Analytical data |
|---|---|---|---|
| C1 | N-[(2-chlorothiazol-5- yl)methyl]-1-methyl- pyrazol-3-amine | 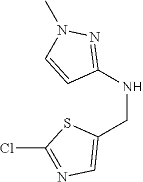 | See example I1 |
| C2 | N-[(6-chloro-3- pyridyl)methyl]-1- methyl-pyrazol-3-amine | 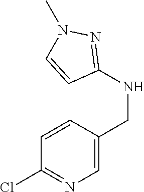 | LC-MS (method A): 223 (M + 1)⁺, retention time 0.62 min |
| C3 | 1-methyl-N-(pyrimidin- 5-ylmethyl)pyrazol-3- amine | 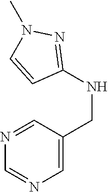 | CAS 1343337-21-2 |
| C4 | 1-methyl-N-(2,2,2- trifluoroethyl)pyrazol-3- amine | 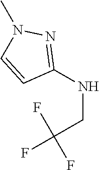 | CAS 1514978-81-4 |
| C5 | 1-methyl-N-(3,3,3- trifluoropropyl)pyrazol- 3-amine | 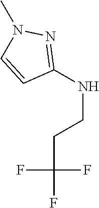 | CAS 1521828-19-2 |
| C6 | 3-[(1-methylpyrazol-3- yl)amino]propanenitrile | 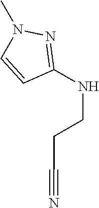 | See example I5 |
| C7 | N-[(2-chlorothiazol-5- yl)methyl]-1-phenyl- pyrazol-3-amine | 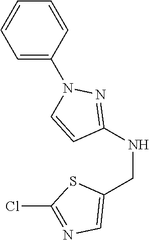 [SMILES: Clc1ncc(CNc2ccn(-c3ccccc3)n2)s1] | ¹H NMR (400 MHz, DMSO-d6) δ 8.19 (s, 1H), 7.68 (d, 2H), 7.59 (s, 1H), 7.39 (m, 2H), 7.12 (m, 1H), 6.31 (d, 1H), 5.81 (s, 1H), 4.47 (d, 2H). |
| C8 | N-[(2-chlorothiazol-5- yl)methyl]-1-ethyl- pyrazol-3-amine | 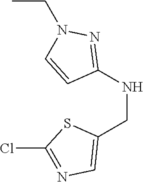 | ¹H NMR (400 MHz, CDCl₃) δ 7.40 (s, 1H), 7.16 (s, 1H), 5.52 (d, 1H), 4.46 (s, 2H), 3.98 (m, 2H), 1.42 (m, 3H). |
| C9 | N-[(2-chlorothiazol-5- yl)methyl]-1,5-dimethyl- pyrazol-3-amine | 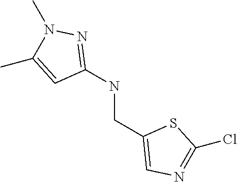 | LC-MS (method A): 243 (M + 1)⁺, retention time 0.65 min |
| C10 | tert-butyl N-(1- methylpyrazol-3-yl)-N- [[2-(trifluoromethyl) thiazol-5-yl]methyl] carbamate | 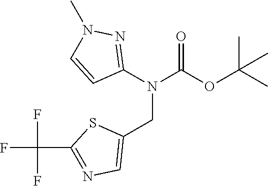 [SMILES: Cn1ccc(N(Cc2cnc(C(F)(F)F)s2)C(=O)OC(C)(C)C)n1] | ¹H NMR (400 MHz, DMSO-d6) δ 8.02 (s, 1H), 7.58 (s, 1H), 6.22 (s, 1H), 5.15 (s, 2H), 3.76 (s, 3H), 1.45 (s, 9H) |
| C11 | N-[(2-chlorothiazol-5- yl)methyl]-1- (difluoromethyl)pyrazol- 3-amine | 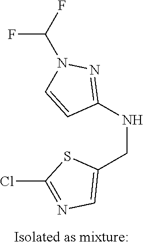 [SMILES: FC(F)n1ccc(NCc2cnc(Cl)s2)n1] | The mixture was used in the next step: NMR of the mixture: ¹H NMR (400 MHz, DMSO-d6) δ 8.17 (s, 1H), 7.88-7.59 (m, 1H), 7.84 (s, 1H), 7.79 (s, 1H), 7.59 (s, 1H), 7.55 (s, 1H), 7.62-7.32 (m, 1H), 6.60 (s, 1H), 6.45 (s, 1H), 5.78 (s, 1H), 5.42 (s, 2H), 5.09 (s, 2H), 4.42 (d, 2H). |
| | | 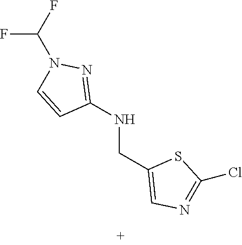 [SMILES: FC(F)n1ccc(NCc2cnc(Cl)s2)n1] | |
| | | 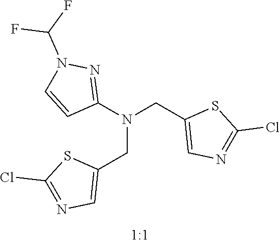 [SMILES: FC(F)n1ccc(N(Cc2cnc(Cl)s2)Cc2cnc(Cl)s2)n1] | |
| C12 | 3-[(1-ethylpyrazol-3- yl)amino]propanenitrile | 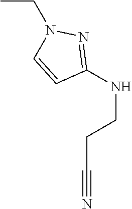 | LC-MS (method A): 164 (M + 1)⁺, retention time 0.35 min |
| C13 | 2-methyl-3-[(1- methylpyrazol-3- yl)amino]propanenitrile | 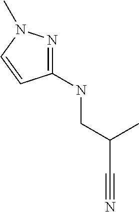 | See example I7 |
| C14 | 2,2-dimethyl-3-[(1- methylpyrazol-3- yl)amino]propanenitrile | 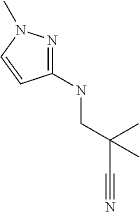 | ¹H NMR (400 MHz, CDCl₃-d) δ ppm 7.12 (s, 1H), 5.58 (s, 1H), 3.88 (m, 1H), 3.88 (m, 1H), 3.72 (s, 3H), 3.33 (m, 2H) |
| C15 | N-(3-furylmethyl)-1- methyl-pyrazol-3-amine | 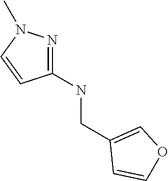 | 1H NMR (400 MHz, DMSO-d6) δ 7.51 (d, 2H), 7.27 (d, 1H), 6.44 (s, 1H), 5.40 (d, 1H), 5.28 (s, 1H), 3.99 (d, 2H), 3.57 (s, 3H). |
| C16 | 1-methyl-N-(3- thienylmethyl)pyrazol-3- amine | 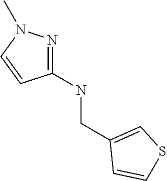 | 1H NMR (400 MHz, DMSO-D6) δ 7.80 (s, 1H), 7.19 (s, 1H), 6.87 (s, 2H), 5.75 (s, 1H), 5.10 (s, 1H), 4.40 (s, 2H), 3.94 (s, 3H). |
| C17 | 1-methyl-N-[[5- (trifluoromethyl)-1,3,4- oxadiazol-2- yl]methyl]pyrazol-3- amine | 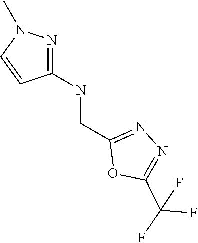 [SMILES: Cn1ccc(NCc2nnc(C(F)(F)F)o2)n1] | 1H NMR (400 MHz, DMSO-d6) δ 7.31 (d, 1H), 6.04 (t, 1H), 5.46 (d, 1H), 4.55 (d, 2H), 3.54 (s, 3H). |
| C18 | N-[(3-chloroisoxazol-5- yl)methyl]-1-methyl- pyrazol-3-amine | 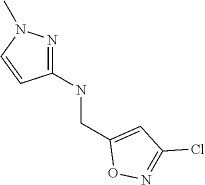 | 1H NMR (400 MHz, DMSO-d6) δ 7.30 (d, 1H), 6.53 (s, 1H), 5.86 (t, 1H), 5.43 (d, 1H), 4.32 (d, 2H), 3.57 (d, 3H). |
| C19 | N-[(4- bromophenyl)methyl]-1- methyl-pyrazol-3-amine | 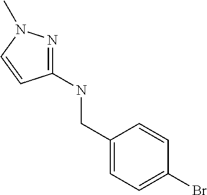 | 1H NMR (400 MHz, DMSO-d6) δ 7.45 (d, 2H), 7.30-7.22 (m, 3H), 5.71 (s, 1H), 5.35 (d, 1H), 4.14 (s, 2H), 3.54 (s, 3H). |
| C20 | N-(isoxazol-4-ylmethyl)- 1-methyl-pyrazol-3- amine | 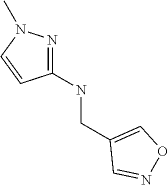 | 1H NMR (400 MHz, DMSO-d6) δ 8.73 (s, 1H), 8.50 (s, 1H), 7.29 (s, 1H), 5.46 (s, 1H), 5.41 (s, 1H), 4.03 (d, J = 6.1 Hz, 2H), 3.58 (d, J = 1.1 Hz, 3H). |
| C21 | 1-methyl-N-[(thiazol-5- yl)methyl]pyrazol-3- amine | 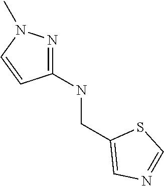 | 1H NMR (400 MHz, DMSO-d6) δ 8.88 (s, 1H), 7.75 (s, 1H), 7.29 (s, 1H), 5.72 (s, 1H), 5.41 (t, 1H), 4.40 (s, 2H), 3.58 (d, 3H). |
| C22 | 1-methyl-N-[(2- bromothiazol-5- yl)methyl]pyrazol-3- amine | 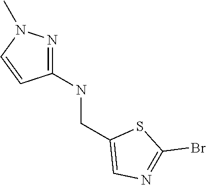 | 1H NMR (400 MHz, DMSO-d6) δ 7.52 (s, 1H), 7.31 (s, 1H), 5.79 (t, 1H), 5.40 (d, 1H), 4.34 (d, 2H), 3.59 (s, 3H). |
| C23 | 1-methyl-N-[(2- methylsulfanylthiazol-5- yl)methyl]pyrazol-3- amine | 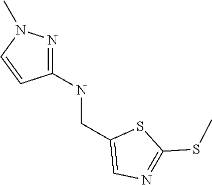 | 1H NMR (400 MHz, DMSO-d6) δ 7.50 (s, 1H), 7.29 (s, 1H), 5.73 (s, 1H), 5.40 (s, 1H), 4.31 (s, 2H), 3.58 (d, 3H), 2.59 (d, 3H). |
| C24 | 1-methyl-N-[(2- methylthiazol-5- yl)methyl]pyrazol-3- amine | 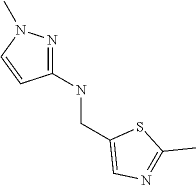 | 1H NMR (400 MHz, DMSO-d6) δ 7.26 (d, 1H), 7.12 (s, 1H), 5.48 (t, 1H), 5.41 (d, 1H), 4.21 (d, 2H), 3.56 (s, 3H), 2.59 (s, 3H). |
| C25 | 1-methyl-N-(2- pyridylmethyl)pyrazol-3- amine | 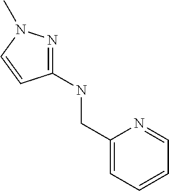 | 1H NMR (400 MHz, DMSO-d6) δ 8.45 (d, 1H), 7.72-7.64 (m, 1H), 7.34 (d, 1H), 7.25 (d, 1H), 7.18 (dd, 1H), 5.70 (t, 1H), 5.38 (d, 1H), 4.27 (d, 2H), 3.54 (s, 3H). |
| C26 | 1-methyl-N-(3- pyridylmethyl)pyrazol-3- amine | 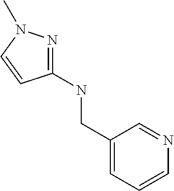 | 1H NMR (400 MHz, DMSO-d6) δ 8.52 (s, 1H), 8.39 (s, 1H), 7.71 (d, 1H), 7.37-7.23 (m, 2H), 5.79 (s, 1H), 5.39 (t, 1H), 4.20 (s, 2H), 3.55 (d, 3H). |
| C27 | N-[(2-chloro-3- pyridyl)methyl]-1- methyl-pyrazol-3-amine | 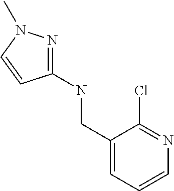 | 1H NMR (400 MHz, DMSO-d6) δ 8.24 (d, 1H), 7.79 (d, 1H), 7.36 (m, 1H), 7.28 (d, 1H), 5.83 (s, 1H), 5.40 (d, 1H), 4.23 (s, 2H), 3.53 (s, 3H). |
| C28 | N-[(4-chloro-3- pyridyl)methyl]-1- methyl-pyrazol-3-amine | 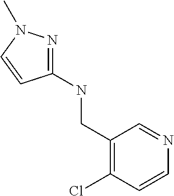 | 1H NMR (400 MHz, DMSO) δ 8.55 (s, 1H), 8.38 (d, 1H), 7.49 (d, 1H), 7.30 (s, 1H), 5.43 (d, 1H), 4.30 (s, 2H), 3.55 (s, 3H). |
| C29 | N-[(5-chloro-3- pyridyl)methyl]-1- methyl-pyrazol-3-amine | 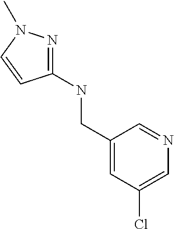 | 1H NMR (400 MHz, DMSO) δ 8.48 (d, 1H), 8.44 (d, 1H), 7.83 (s, 1H), 7.28 (d, 1H), 5.83 (t, 1H), 5.41 (d, 1H), 4.22 (d, 2H), 3.54 (s, 3H). |
| C30 | N-[(5,6-dichloro-3- pyridyl)methyl]-1- methyl-pyrazol-3-amine | 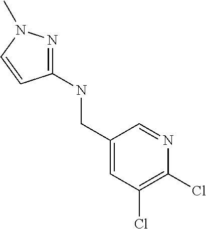 [SMILES: Cn1ccc(NCc2cnc(Cl)c(Cl)c2)n1] | 1H NMR (400 MHz, DMSO) δ 8.69 (s, 1H), 8.42 (s, 1H), 7.28 (d, 1H), 5.91 (t, 1H), 5.42 (d, 1H), 4.34 (d, 2H), 3.53 (s, 3H). |
| C31 | N-[(6-fluoro-3- pyridyl)methyl]-1- methyl-pyrazol-3-amine | 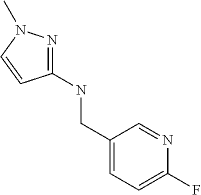 | 1H NMR (400 MHz, DMSO-d6) δ 8.16 (s, 1H), 7.97-7.85 (m, 1H), 7.30 (d, 1H), 7.08 (m, 1H), 5.88 (s, 1H), 5.43 (t, 1H), 4.21 (s, 2H), 3.65-3.47 (s, 3H). |
| C32 | 5-[[(1-methylpyrazol-3- yl)amino]methyl]pyridine- 2-carbonitrile | 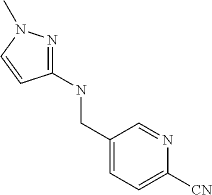 [SMILES: Cn1ccc(NCc2ccc(C#N)nc2)n1] | ¹H NMR (400 MHz, DMSO) δ 8.68 (s, 1H), 7.93 (d, 2H), 7.27 (s, 1H), 5.90 (t, 1H), 5.40 (d, 1H), 4.29 (m, 2H), 3.53 (s, 3H). |
| C33 | N-[(2-chloro-4- pyridyl)methyl]-1- methyl-pyrazol-3-amine | 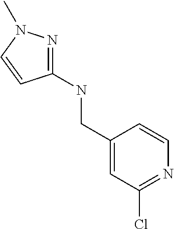 | 1H NMR (400 MHz, DMSO) δ 8.26 (t, 1H), 7.40 (s, 1H), 7.33 (d, 1H), 7.28 (d, 1H), 5.91 (s, 1H), 5.39 (d, 1H), 4.23 (s, 2H), 3.52 (d, 3H). |
| C34 | N-[(2-chloropyrimidin-5- yl)methyl]-1-methyl- pyrazol-3-amine | 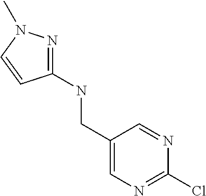 | 1H NMR (400 MHz, DMSO) δ 8.67 (d, 2H), 7.28 (s, 1H), 5.83 (t, 1H), 5.48-5.35 (m, 1H), 4.20 (d, 2H), 3.54 (s, 3H). |
| C35 | N-[(5-chloropyrazin-2- yl)methyl]-1-methyl- pyrazol-3-amine | 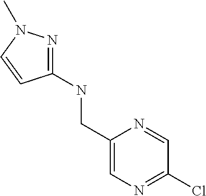 | 1H NMR (400 MHz, DMSO) δ 8.33 (s, 1H), 8.03 (d, 1H), 7.28 (d, 1H), 5.84 (t, 1H), 5.41 (d, 1H), 4.22 (d, 2H), 3.55 (s, 3H). |
| C36 | N-(3,6-dihydro-2H- pyran-5-ylmethyl)-1- methyl-pyrazol-3-amine | 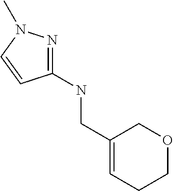 | ¹H NMR (400 MHz, CDCl₃) δ 7.11 (d, 1H), 5.78 (d, 1H), 5.51 (d, 1H), 4.10 (d, 1H), 3.74 (d, 4H), 3.65 (s, 2H), 2.14 (s, 2H). |
| C37 | 1-methyl-N- (tetrahydrofuran-3- ylmethyl)pyrazol-3- amine | 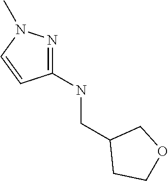 | 1H NMR (400 MHz, DMSO-d6) δ 7.25 (s, 1H), 5.35 (d, 1H), 5.14 (s, 1H), 3.72-3.64 (m, 2H), 3.62- 3.49 (m, 4H), 3.39 (m, 1H), 2.98-2.86 (m, 2H), 2.40 (m, 1H), 1.89 (m, 1H), 1.52 (m, 1H). |

TABLE D
Examples of intermediates of formula (VIIIa) and (VIIIb)

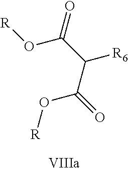

| No. | IUPAC name | R | R₆ | Analytical data |
|---|---|---|---|---|
| D1 | bis(2,4,6- trichlorophenyl) 2- (3,5-dichlorophenyl) propanedioate | 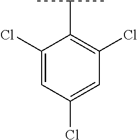 | 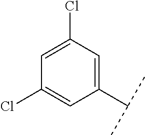 | See example I1 |
| D2 | bis(2,4,6- trichlorophenyl) 2- (4-bromophenyl) propanedioate | 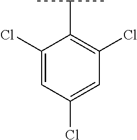 | 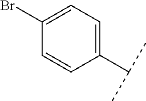 | ¹H NMR (400 MHz, CDCl₃) δ ppm 7.42 (s, 4 H), 7.55 (d, 2 H), 7. 64 (d, 2 H), 5.31 (s, 1H). |
| D3 | bis(2,4,6- trichlorophenyl) 2- (3-bromophenyl) propanedioate | 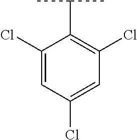 | 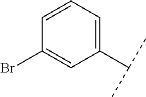 | ¹H NMR (400 MHz, CDCl₃) δ ppm 7.82 (s, 1H), 7.58(m, 2H), 7.38(m, 5H), 5.30 (s,1H). |
| D4 | bis(2,4,6- trichlorophenyl) 2- (6-bromo-2- naphthyl) propanedioate | 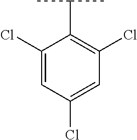 | 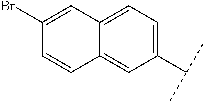 | ¹H NMR (400 MHz, CDCl₃) δ ppm 8.08 (m, 2H), 7.82 (m, 1H), 7.75 (m, 2H), 7.60 (m,1H), 7.48 (s, 4H), 5.46 (s, 1H). |
| D5 | bis(phenyl) 2-[3- bromo-5- (trifluoromethyl) phenyl] propanedioate | 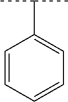 | 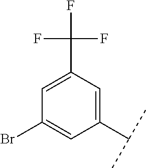 | See example I2 |
| D6 | bis(phenyl) 2-[3-[3- chloro-5- (trifluoromethyl)-2- pyridyl]phenyl] propanedioate | 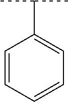 | 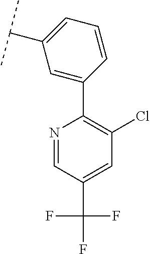 | See example I3 |
| D7 | bis(2,4,6- trichlorophenyl) 2- indan-5- ylpropanedioate | 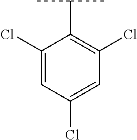 | 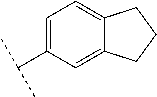 | 1H NMR (400 MHz, DMSO- d6) δ 7.84 (s, 2H), 7.51 (s, 2H), 7.47 (s, 1H), 7.37 (d, 1H), 7.29 (d, 1H), 6.04 (s, 1H), 2.85 (d, 4H), 1.99 (m, 2H) |
| D8 | bis(2,4,6- trichlorophenyl) 2- [3- (trifluoromethoxy) phenyl]propanedioate | 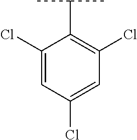 | 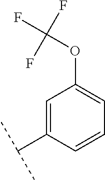 | 1H NMR (400 MHz, CDCl₃) δ 7.57 (d, 1H), 7.53 (s, 1H), 7.49 (t, 1H), 7.37 (s, 3H), 7.30 (d, 1H), 5.33 (s, 1H). |
| D9 | bis(2,4,6- trichlorophenyl) 2- tetralin-6- ylpropanedioate | 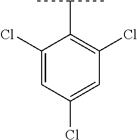 | 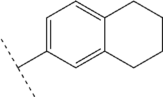 | 1H NMR (400 MHz, CDCl₃) δ 7.36 (s, 4H), 7.30 (d, 2H), 7.12 (d, 1H), 5.24 (s, 1H), 2.78 (s, 4H), 1.80 (t, 4H). |
| D10 | bis(2,4,6- trichlorophenyl) 2- [3- (trifluoromethylsulfanyl) phenyl]propane dioate | 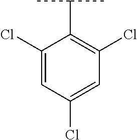 | 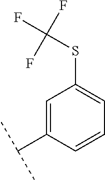 | 1H NMR 400 MHz, CDCl₃δ 7.94 (s, 1H), 7.78 (d, 1H), 7.74 (d, 1H), 7.53 (t, 1H), 7.37 (s, 4H), 5.34 (s, 1H). |
| D11 | bis(2,4,6- trichlorophenyl) 2- [3-chloro-5- (trifluoromethoxy) phenyl]propanedioate | 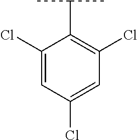 | 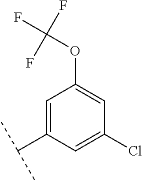 | 1H NMR (400 MHz, CDCl₃) δ 7.59 (d, 1H), 7.44 (s, 1H), 7.38 (s, 4H), 7.32 (s, 1H), 5.28 (s, 1H). |
| D12 | | 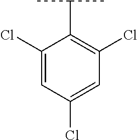 | 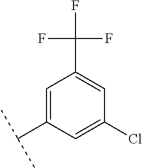 | 1H NMR (400 MHz, DMSO- d6) δ 7.73 (s, 1H), 7.68 (s, 1H), 7.63 (s, 1H), 7.52 (s, 4H), 3.86 (s, 1H). |
| D13 | bis(2,4,6- trichlorophenyl) 2- [3-chloro-5- (trifluoromethyl) phenyl]propanedioate | 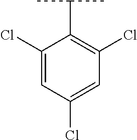 | 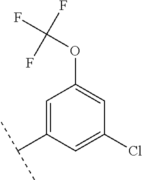 | 1H NMR (400 MHz, CDCl₃) δ 7.59 (d, 1H), 7.44 (s, 1H), 7.38 (s, 4H), 7.32 (s, 1H), 5.28 (s, 1H). |
| D14 | diphenyl 2-(3- chloro-5-iodo- phenyl)propanedioate | 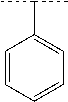 | 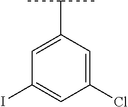 | ¹H NMR (400 MHz, CDCl₃): δ 7.88-7.81 (m, 1H), 7.77 (t, 1H), 7.59 1H), 7.42-7.38 (m, 3H), 7.30-7.29 (m, 1H), 7.26 (s, 2H), 7.14-7.11 (m, 4H), 5.00 (s, 1H). |
| D15 | diphenyl 2- (cyclohexylmethyl) propanedioate | 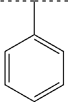 | 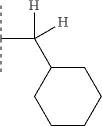 | ¹H NMR (400 MHz, CDCl₃): δ 7.42-7.37 (m, 4H), 7.28- 7.24 (m, 2H), 7.14-7.12 (m, 4H), 3.94 (t, 1H), 2.07 (t, 2H), 1.86 (d, 2H), 1.78-1.67 (m, 3H), 1.52-1.44 (m, 1H), 1.30-1.20 (m, 3H), 1.08- 1.01 (m, 2H). |

TABLE E
Examples of intermediates of formula (IX) and (IXa)

| No. | IUPAC name | Structures | Analytical data |
|---|---|---|---|
| E1 (2.003) | ethyl 3,3-dichloro-2-(3,5- dichlorophenyl)prop-2- enoate | 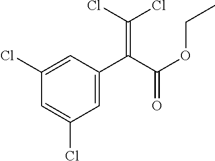 [SMILES: CCOC(=O)C(=C(Cl)Cl)c1cc(Cl)cc(Cl)c1] | See example I4 |
| E2 (2.001) | 3,3-dichloro-2-(3,5- dichlorophenyl)prop-2-enoic acid | 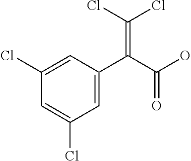 [SMILES: O=C(O)C(=C(Cl)Cl)c1cc(Cl)cc(Cl)c1] | See example I4 |
| E4 (2.030) | ethyl 3,3-dichloro-2-[3- (trifluoromethyl)phenyl]prop- 2-enoate | 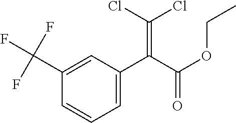 [SMILES: CCOC(=O)C(=C(Cl)Cl)c1cccc(C(F)(F)F)c1] | ¹H NMR (400 MHz, CDCl3) δ ppm 7.5-7.8 (m, 4H), 4.28 (m, 2H), 1.3 (m, 3H). |
| E5 (2.028) | 3,3-dichloro-2-[3- (trifluoromethyl)phenyl]prop- 2-enoic acid | 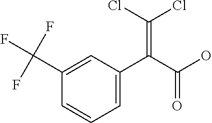 [SMILES: O=C(O)C(=C(Cl)Cl)c1cccc(C(F)(F)F)c1] | ¹H NMR (300 MHz, DMSO) δ ppm 14.05 (sb, 1H), 7.8 (s, 1H), 7.7 (m, 3H). |

### BIOLOGICAL EXAMPLES

Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm:

### A1, A3, A4, A7, A8, A9, A10, A13, A17, A24, A25, A27, A28, A30, A31, A47, A60, B2, B3, B4, B8, B10, B13, B14

### Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm:

### A30, A39, A46, B2, B3, B4, B8, B10, B11, B12, B13, B14, B15

Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation. The following compounds gave an effect of at least 80% in at least one of the two categories (mortality or growth inhibition) at an application rate of 200 ppm:

### A33, A37, A39, A40, A43, A46, A47, A48, A49

Myzus persicae (Green Peach Aphid): Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm:

### A7, A14, A16, B12, B18, B21

Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm:

### A3, A4, A7, A13, A24, A28, A29, A30, A31

Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm:

### A1, A3, A4, A7, A8, A9, A13, A17, A23, A24, A25, A27, A28, A29, A30, A31, A33, A47, B4, B8, B13, B14

Spodoptera littoralis (Egyptian Cotton Leaf Worm)

Test compounds were applied by pipette from 10′000 ppm DMSO stock solutions into 24-well plates and mixed with agar. Lettuce seeds were placed onto the agar and the multi well plate was closed by another plate which contained also agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

The following compounds gave an effect of at least 80% in at least one of the three categories (mortality, anti-feeding, or growth inhibition) at a test rate of 12.5 ppm:

### A24, A50

Thrips tabaci (Onion Thrips) Feeding/Contact Activity

Sunflower leaf discs were placed on agar in 24-well microtiter plates and sprayed with aqueous test solutions prepared from 10′000 ppm DMSO stock solutions. After drying the leaf discs were infested with a thrips population of mixed ages. The samples were assessed for mortality 6 days after infestation.

The following compounds resulted in at least 80% mortality at an application rate of 200 ppm:

### B2, B4, B8, B13

What is claimed is:

1. A compound of formula I, 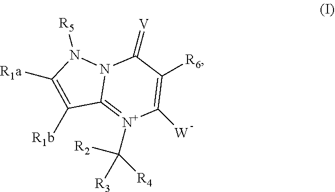
wherein
W is S or O;
V is S or O;
R₁ₐ and R_{1b} are, independently, hydrogen, halogen, amino, hydroxyl, C₁-C₆alkyl, C₁-C₆ haloalkyl, C₁-C₆haloalkoxy, C₁-C₆ alkoxy, or cyano;
R₂ is hydrogen, halogen, hydroxyl, amino, cyano, C₁-C₆ alkyl, mono- or poly-substituted C₁-C₆ alkyl wherein the substituent is independently selected from the group consisting of halogen, hydroxyl, amino, cyano, nitro, C₁-C₆ haloalkoxy, C₁-C₆ alkoxy, triazole, pyrazole, imidazole and tetrazole, wherein said triazole, pyrazole, imidazole and tetrazole can be mono- or polysubstituted by substituents independently selected from the group consisting of halogen, C₁-C₄ alkyl, C₁-C₄ alkoxy, C₁-C₄ haloalkyl and cyano;
R₃ is hydrogen or C₁-C₆ alkyl;
R₄ is hydrogen or a 5 or 6 membered heteroaromatic ring Y, optionally independently substituted with a substituent from the group selected from U, wherein Y is a ring selected from Y1 to Y29 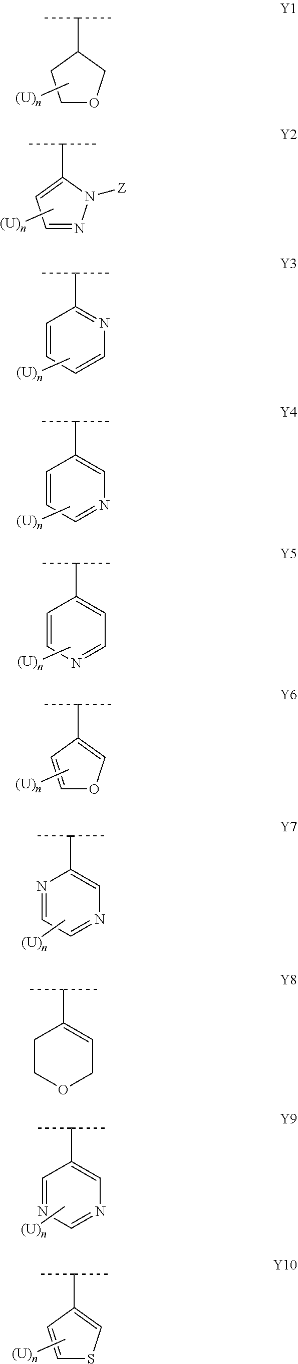 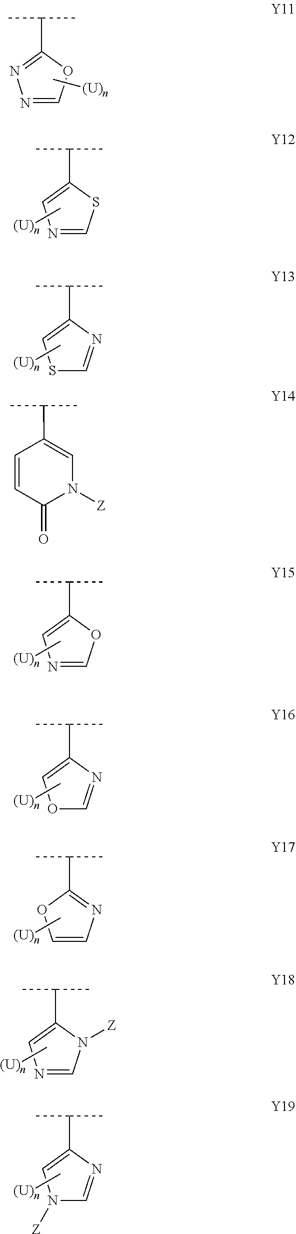 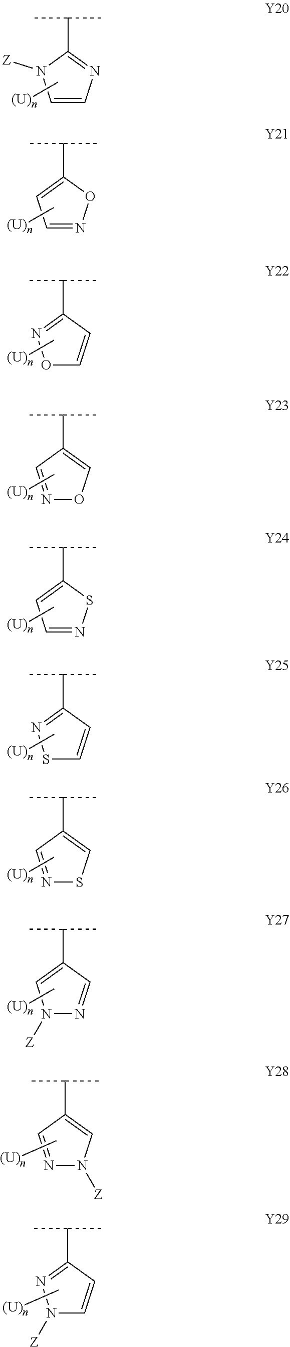
n is 0, 1, 2 or 3;
Z is hydrogen, cyano, nitro, hydroxyl, C₁-C₄ alkyl, C₁-C₄ haloalkyl, C₁-C₄ alkoxy or C₁-C₄ haloalkoxy;
U is independently selected from the group consisting of halogen, cyano, nitro, hydroxyl, amino, C₁-C₄ alkyl, C₁-C₄ haloalkyl, C₁-C₄ alkoxy, C₁-C₄ haloalkoxy, C₁-C₄ haloalkoxy-C₁-C₄ alkyl, C₁-C₄ alkoxy-C₁-C₄ alkyl, C₁-C₄ alkylsulfanyl, C₁-C₄ alkylsulfinyl, C₁-C₄ alkylsulfonyl, C₁-C₄haloalkylsulfanyl, C₁-C₄haloalkylsulfinyl, C₁-C₄haloalkylsulfonyl, and cyclopropyl;
R₅ is C₁-C₆ alkyl, C₃-C₆ cycloalkyl, C₁-C₆ haloalkyl, or C₁-C₆ alkoxy; or
R₅ is phenyl, the ring system of either can be mono- or polysubstituted by substituents independently selected from halogen, C₁-C₄ alkyl, C₁-C₄haloalkyl, C₁-C₄ alkoxy and C₁-C₄ haloalkoxy; and
R₆ is a 5 to 12 membered aromatic ring, which can be monocyclic or polycyclic, which ring system can be mono- or polysubstituted by substituents independently selected from the group U₂; or
R₆ is a 3 to 12 membered heteroaromatic ring or saturated or partially saturated heterocyclic ring, each of which ring system can be monocyclic or polycyclic, which ring system can contain 1 to 4 hetero atoms selected from the group consisting of nitrogen, oxygen and sulfur, with the proviso that each ring system cannot contain more than 2 oxygen atoms or more than 2 sulfur atoms, wherein the nitrogen heteroatom can be substituted by Z and said 3 to 12-membered ring system can be mono- or polysubstituted by substituents independently selected from the group U₂; or
R₆ is hydrogen, amino, halogen, cyano, C₁-C₆ haloalkoxy, C₁-C₆ alkoxy, C₁-C₄ alkylsulfanyl, C₁-C₄ alkylsulfinyl, C₁-C₄ alkylsulfonyl, C₁-C₄haloalkylsulfanyl, C₁-C₄haloalkylsulfinyl, C₁-C₄haloalkylsulfonyl, C₂-C₆ alkenyl, C₂-C₆ haloalkenyl, C₂-C₆ alkynyl, C₂-C₆ haloalkynyl, benzyl optionally mono- or poly-substituted by a halogen (in case of polysubstitution, can be the same of different) or —C(O)R₇. or
R₆ is C₁-C₆ alkyl, which is optionally mono- or polysubstituted by substituents independently selected from the group U₃, or
R₆ is C₃-C₆ cycloalkyl, which is optionally mono- or polysubstituted by substituents independently selected from the group U; wherein
U₂ is halogen, nitro, cyano, amino, hydroxyl, —SCN, —CO₂H, C₁-C₆ alkyl, C₃-C₆ cycloalkyl, C₃-C₆ halocycloalkyl, C₃-C₆ cycloalkyl-C₁-C₄ alkyl, C₃-C₆ halocycloalkyl-C₁-C₄ alkyl, C₁-C₆ haloalkyl, C₁-C₆ alkoxy, C₁-C₄ alkoxy-C₁-C₄ alkyl, C₁-C₄ alkoxy-C₁-C₄ alkoxy, cyano-C₁-C₄ alkyl, cyano-C₁-C₄ haloalkyl, C₂-C₆ alkenyl, C₂-C₆ haloalkenyl, C₂-C₆ alkynyl, C₂-C₆ haloalkynyl, C₁-C₆ haloalkoxy, C₁-C₄ haloalkoxy-C₁-C₄ alkyl, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, C₁-C₆ alkylcarbonyl, C₁-C₆ alkoxycarbonyl, C₁-C₆ haloalkylcarbonyl, C₁-C₆ haloalkoxycarbonyl, (C₁-C₆ alkyl)NH, (C₁-C₆ alkyl)₂N, (C₃-C₆ cycloalkyl)NH, (C₃-C₆ cycloalkyl)₂N, C₁-C₆ alkylcarbonylamino, C₃-C₆ cycloalkylcarbonylamino, C₃-C₆ haloalkylcarbonylamino, C₃-C₆ halocycloalkylcarbonylamino, C₁-C₆ alkylaminocarbonyl, C₃-C₆ cycloalkylaminocarbonyl, C₁-C₆ haloalkylaminocarbonyl, C₃-C₆ halocycloalkylaminocarbonyl, C₃-C₆ cycloalkylcarbonyl, C₃-C₆ halocycloalkylcarbonyl, —SF₅ or —C(O)NH₂;
U₃ is halogen, nitro, cyano, amino, hydroxyl, C₁-C₆ alkyl, C₃-C₆ cycloalkyl, C₃-C₆ halocycloalkyl, C₃-C₆ cycloalkyl-C₁-C₄ alkyl, C₃-C₆ halocycloalkyl-C₁-C₄ alkyl, C₁-C₆ haloalkyl, C₁-C₆ alkoxy, C₁-C₄ alkoxy-C₁-C₄ alkyl, C₁-C₄ alkoxy-C₁-C₄ alkoxy, cyano-C₁-C₄ alkyl, cyano-C₁-C₄ haloalkyl, C₂-C₆ alkenyl, C₂-C₆ haloalkenyl, C₂-C₆ alkynyl, C₂-C₆ haloalkynyl, C₁-C₆ haloalkoxy, C₁-C₄ haloalkoxy-C₁-C₄ alkyl, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, C₁-C₆ alkylcarbonyl, C₁-C₆ alkoxycarbonyl, C₁-C₆ haloalkylcarbonyl or C₁-C₆ haloalkoxycarbonyl; or
U₃ is a 5 to 6 membered aromatic ring, heteroaromatic ring, or saturated or partially saturated carbocyclic or heterocyclic ring (wherein the heteroatomatic and heterocyclic rings can contain 1 to 4 hetero atoms selected from the group consisting of nitrogen substituted or not, oxygen and sulfur, with the proviso that each ring system cannot contain more than 2 oxygen atoms or more than 2 sulfur atoms), wherein the said 5 to 6-membered ring system can be mono- or polysubstituted by substituents independently selected from the group U; and
R₇ is hydrogen, amino, halogen, cyano, C₁-C₆ alkyl, C₁-C₆ haloalkyl, C₁-C₆ alkoxy, C₁-C₆ haloalkoxy, C₁-C₄ haloalkoxy C₁-C₄ alkyl, C₁-C₆ alkoxy-C₁-C₆ alkyl, C₂-C₆ alkenyl, C₂-C₆ haloalkenyl, C₂-C₆ alkynyl or C₂-C₆ haloalkynyl; or
R₇ is a 5 to 6 membered aromatic ring, heteroaromatic ring, or saturated or partially saturated carbocyclic or heterocyclic (wherein the heteroatomatic and heterocyclic rings can can contain 1 to 4 hetero atoms selected from the group consisting of nitrogen substituted or not, oxygen and sulfur, with the proviso that each ring system cannot contain more than 2 oxygen atoms and more than 2 sulfur atoms), wherein the said 5 to 6-membered ring system can be mono- or polysubstituted by substituents independently selected from the group U; or an agrochemically acceptable salt, stereoisomer, enantiomer, tautomer or N-oxide thereof

2. The compound according to claim 1 wherein R₄ is hydrogen or a 5 or 6 membered heteroaromatic ring selected from Y4, Y9, and Y12, U is selected from the group consisting of halogen, and trifluoromethyl, and n is 0, 1.

3. The compound according to claim 1, wherein R₆ is hydrogen, iodine, —C(O)R₇ (wherein R₇ is trifluoromethyl or phenyl), phenyl optionally mono- or poly-substituted by the group consisting of halogen and trifluoromethyl, naphthyl optionally substituted by a halogen (in case of polysubstitution, can be the same or different), pyridylphenyl optionally mono- or poly-substituted by substituents independently selected from halogen and trifluoromethyl, or C₁-C₄ alkyl, which is optionally mono- or poly-substituted by substituents independently selected from chlorine and fluorine.

4. The compound according to claim 1, wherein W and V are each O, R₁ₐ and R_{1b} are each hydrogen; R₂ is selected from hydrogen, trifluoromethyl, trifluoroethyl and cyanomethyl; R₃ is hydrogen; R₄ is hydrogen or a 5 or 6 membered heteroaromatic ring selected from Y1, Y3, Y4, Y5, Y7, Y9, Y12, Y18, Y21 and Y23, wherein Z is C₁-C₄ alkyl, U is selected from the group consisting of halogen, C₁-C₄haloalkyl, C₁-C₄ alkoxyl, cyano, C₁-C₄ alkylsulfanyl and C₁-C₄ alkylsulfonyl, and n is 0, 1; preferably wherein Z is methyl, U is selected from the group consisting of halogen, trifluoromethyl, methoxy, cyano, methylsulfanyl and methylsulfonyl, and n is 0, 1; R₅ is methyl, ethyl, trifluoroethyl or cyclopropyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which is optionally mono- or poly-substituted by substituents independently selected from a halogen (in case of polysubstitution, can be the same or different), or C₃-C₆ cycloalkyl.

5. The compound according to claim 1, wherein W and V are each O, R₁ₐ and R_{1b} are each hydrogen; R₂ is selected from trifluoromethyl, trifluoroethyl and cyanomethyl; R₃ is hydrogen; R₄ is hydrogen; R₅ is methyl, ethyl, trifluoroethyl, or cyclopropyl; and R₆ is hydrogen, halogen, —C(O)R₇ (wherein R₇ is C₁-C₆ haloalkyl, phenyl or halophenyl), phenyl optionally mono- or poly-substituted by the group consisting of halogen, C₁-C₆ haloalkyl, C₁-C₆ haloalkoxyl and C₁-C₄ haloalkylsulfanyl, benzyl optionally mono- or poly-substituted by a halogen (in case of polysubstitution, can be the same or different), naphthyl optionally substituted by a halogen (in case of polysubstitution, can be the same or different), pyridylphenyl optionally mono- or poly-substituted by substituents independently selected from halogen and C₁-C₄ haloalkyl, C₁-C₄ alkyl, which is optionally mono- or poly-substituted by substituents independently selected from a halogen (in case of polysubstitution, can be the same or different), or C₃-C₆ cycloalkyl.

6. A compound of formulae IXa, IXb and IXc 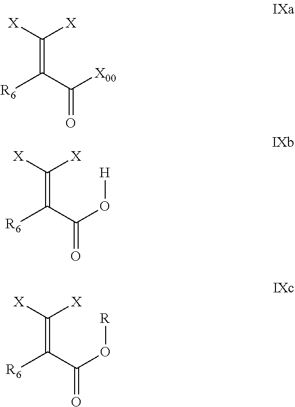
where R₆ in each of IXa, IXb and IXc is 3,5-dichloro phenyl or 3-trifluormethylphenyl; X in each of IXa, IXb and IXc is a halogen atom (preferably chlorine; R in formula IXc is methyl, or ethyl, and X₀₀ is a halogen atom, or an iso-urea-containing compound, such as 1,3-dicyclohexyl-isourea-2-yl; and acceptable salts, stereoisomers, enantiomers, tautomers and N-oxides.

7. A compound of formula X 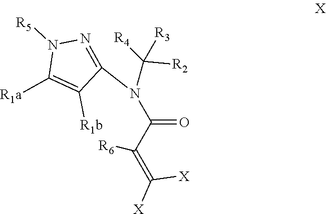
where R₁ₐ, R_{1b}, R₂, R₃, R₄, R₅ and R₆ are as defined in claim 1 and X is a halogen (preferably Cl); and acceptable salts, stereoisomers, enantiomers, tautomers and N-oxides.

8. A compound of formula XXI 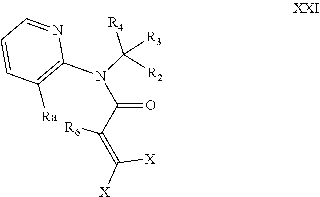
R₂, R₃, R₄, and R₆ are as defined in claim 1, Ra is hydrogen or methyl, and X is a halogen (preferably Cl); and acceptable salts, stereoisomers, enantiomers, tautomers and N-oxides.

9. A compound of formula XI 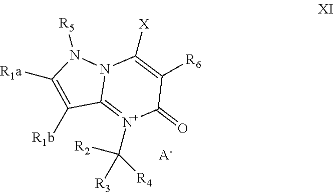
where R₁ₐ, R_{1b}, R₂, R₃, R₄, R₅ and R₆ are as defined in claim 1, X is a halogen (preferably Cl), and A⁻ is an anion, preferably selected from AlCl₄⁻ and Cl⁻; and acceptable salts, stereoisomers, enantiomers, tautomers and N-oxides.

10. A compound of formulae XXII and XXIV 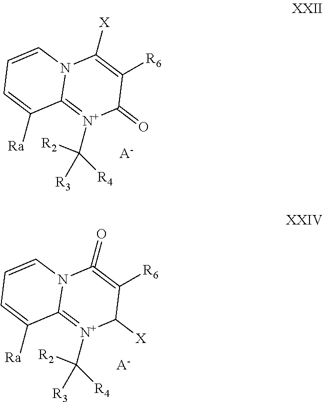
where R₂, R₃, R₄, and R₆ are, independent of formula XXII and XXIV, as defined in claim 1, Ra is hydrogen or methyl, X is a halogen (preferably Cl), and A⁻ is an anion, preferably selected from AlCl₄⁻ and Cl⁻; and acceptable salts, stereoisomers, enantiomers, tautomers and N-oxides.

11. A a process for preparing a compound of formula Ib, wherein R₁ₐ, R_{1b}, R₂, R₃, R₄, R₅ and R₆ are as defined in claim 1, by
(i) reaction of compound VI (where R₁ₐ, R_{1b}, R₂, R₃, R₄, and R₅ are as defined in any one of claims 1 to 5) with a compound of formula VIIIa wherein R is aryl or alkyl; 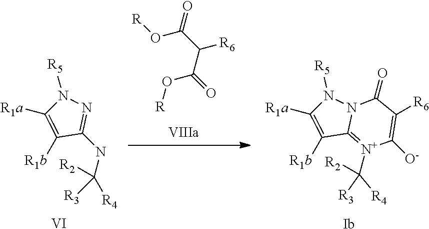
or
(ii) reaction of compound of formula XI, wherein R₁ₐ, R_{1b}, R₂, R₃, R₄, R₅ and R₆ are as defined in any one of claims 1 to 5 and X is halogen, such as chlorine, and A⁻ is an anion, such as for example AlCl₄⁻ or Cl⁻; 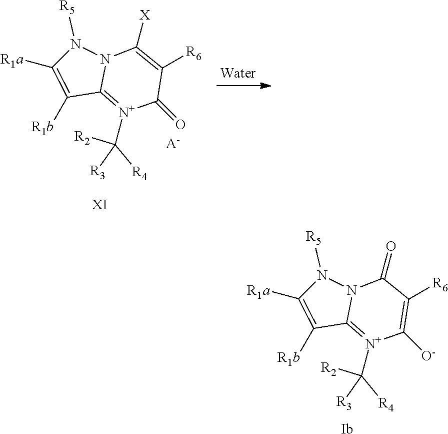
or
(iii) reacting compounds of formula Id (where R₁ₐ, R_{1b}, R₂, R₃, R₄, R₅ and R₆ are as defined in any one of claims 1 to 5), wherein X is a leaving group with compounds of formula XIIa, wherein Y_{b1} can be a boron-derived functional group; or reacting compounds of formula Id, wherein X is a leaving group with compounds of formula XIIb, wherein Yb2 is a trialkyl tin derivative.

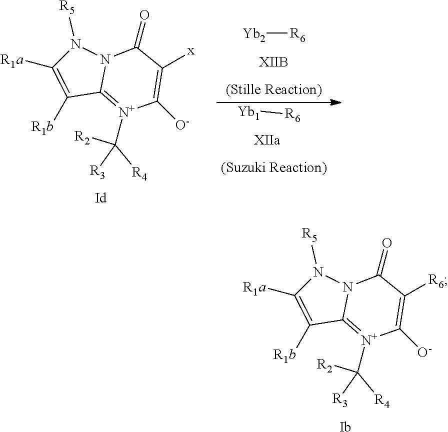

12. A pesticidal composition comprising a compound of formula I defined in claim 1, one or more formulation additives and a carrier.

13. A combination of active ingredients comprising a compound of formula I defined in claim 1, and one or more further active ingredients.

14. A method of controlling insects, acarines, nematodes or molluscs which comprises applying an insecticidally, acaricidally, nematicidally or molluscicidally effective amount of a compound of formula I defined in claim 1.

15. A plant propagation material comprising by way of treatment or coating one or more compounds of formula I defined in claim 1, optionally also comprising a colour pigment.

16. A method of controlling insects, acarines, nematodes or molluscs which comprises a composition containing a compound of formula I defined in claim 1, to a pest, a locus of pest, preferably a plant, to a plant susceptible to attack by a pest or to plant propagation material thereof, such as a seed, provided if the control were on a human or animal body, then it is non-therapeutical.
